(19) United States
(12) Patent Application Publication
(10) Pub. No.: US 2024/0401121 A1
(43) Pub. Date: Dec. 5, 2024

(54) UNIVERSAL HAIRPIN PRIMER SYSTEM FOR QUANTIFICATION OF MICRORNA

(71) Applicant: The University of Chicago, Chicago, IL (US)

(72) Inventor: Tong-Chuan HE, Chicago, IL (US)

(21) Appl. No.: 18/694,220

(22) PCT Filed: Sep. 28, 2022
(86) PCT No.: PCT/US22/77175

§ 371 Date: Mar. 21, 2024

Related U.S. Application Data
(60) Provisional application No. 63/392,562, filed on Jul. 27, 2022.
(60) Provisional application No. 63/250,438, filed on Sep. 30, 2021.

(51) Int. Cl. C12Q 1/6853

(52) U.S. Cl. CPC C12Q 1/6853

(57) ABSTRACT

The disclosure is directed to universal hairpin primer (UHP) nucleic acid molecules for quantifying RNA, including mature microRNA (miRNA), messenger RNA (mRNA), and long noncoding RNA (lncRNA), as well as systems and methods for using same. The UHP nucleic acid molecules comprise a stem-loop structure and a degenerate nucleic acid sequence of 2-10 (e.g., 2-6) nucleotides at the 3′ end, wherein the degenerate nucleic acid sequence hybridizes to the 3′-end of an RNA molecule. The RNA quantification analysis can be carried out by using either the conventional SYBR Green system or the cost-effective universal TaqMan probe-based RT-qPCR system.

### STATEMENT OF RELATED APPLICATIONS

This application claims priority to U.S. Provisional Patent Application No. 63/250,438, filed Sep. 30, 2021, and to U.S. Provisional Patent Application No. 63/392,562, filed Jul. 27, 2022, the entire contents of which are incorporated herein by reference for all purposes.

### SEQUENCE LISTING

The computer readable sequence listing filed herewith, titled “UCHI-39811-601_SQL”, created Sep. 28, 2022, having a file size of 206,129 bytes, is hereby incorporated by reference in its entirety.

### BACKGROUND

Reverse transcriptase-quantitative PCR (RT-qPCR or qPCR) is a commonly-used tool to quantify gene expression in life science. Among various RT-qPCR systems, SYBR Green-based qPCR is the most commonly used method to quantify coding and noncoding transcript expression due to its sensitivity and low cost, although it may lack specificity with limited detection ranges. On the contrary, fluorescent probe-based (e.g., TaqMan) qPCR offers high sensitivity and specificity with broad detection ranges, but there is high cost associated with fluorescent probe synthesis. Accordingly, there remains a need for cost-effective tools for quantification of coding and noncoding RNA transcripts with high sensitivity and specificity.

### BRIEF SUMMARY OF THE DISCLOSURE

The disclosure provides a primer nucleic acid molecule comprising a stem-loop structure and a degenerate nucleic acid sequence of 2-10 (e.g., 2-6) nucleotides at the 3′ end, wherein the degenerate nucleic acid sequence hybridizes to the 3′-end of an RNA molecule.

The disclosure also provides a composition comprising a mixture of two or more primer nucleic acid molecules, wherein each primer nucleic acid molecule comprises a stem-loop structure and a degenerate nucleic acid sequence of 2-10 (e.g., 2-6) nucleotides at the 3′ end, wherein the degenerate nucleic acid sequence hybridizes to the 3′-end of an RNA (miRNA) molecule.

Also provided is a system for quantifying RNA in a sample, which comprises: (a) a primer nucleic acid molecule, or a mixture of primer nucleic acid molecules, wherein each primer nucleic acid molecule comprises a stem-loop structure and a degenerate nucleic acid sequence of 2-10 (e.g., 2-6) nucleotides at the 3′ end, wherein the degenerate nucleic acid sequence hybridizes to the 3′-end of an RNA molecule; (b) a reverse transcriptase; and (c) deoxyribonucleotide triphosphates (dNTPs). A method for quantifying miRNA using the aforementioned system also is described.

The primer nucleic acid molecules, compositions, and systems described herein can bind to coding and noncoding RNA molecules, including micro RNA (miRNA), long noncoding RNA (lncRNA), and messenger RNA (mRNA), and can therefore be used for quantification of each of these RNA types.

In some aspects, provided herein is primer nucleic acid molecule comprising a stem-loop structure and a degenerate nucleic acid sequence of 2-10 nucleotides at the 3′ end, wherein the degenerate nucleic acid sequence hybridizes to the 3′-end of a ribonucleic acid (RNA) molecule. In some embodiments, the RNA molecule is a mature microRNA (miRNA), a messenger RNA (mRNA), or a long noncoding RNA (lncRNA) molecule. In some embodiments, the degenerate nucleic acid sequence comprises 2, 3, 4, or 6 nucleotides. In some embodiments, the degenerate nucleic acid sequence comprises 4 nucleotides. In some embodiments, the stem comprises 14 base pairs and the loop comprises 16 nucleotides.

In some aspects, provided herein is a composition comprising a mixture of two or mor primer nucleic acid molecules as described herein. In some embodiments, provided herein is a composition comprising a mixture of two or more primer nucleic acid molecules. In some embodiments, each primer nucleic acid molecule comprises a stem-loop structure and a degenerate nucleic acid sequence of 2-10 nucleotides at the 3′ end, and the degenerate nucleic acid sequence hybridizes to the 3′-end of a ribonucleic acid (RNA) molecule. In some embodiments, the RNA molecule is a mature microRNA (miRNA), a messenger RNA (mRNA), or a long noncoding RNA (lncRNA) molecule. In some embodiments, the degenerate nucleic acid sequence comprises 2, 3, 4, or 6 nucleotides. In some embodiments, the composition comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and/or (iii) one or more primer nucleic acid molecules having a generate nucleic acid sequence of 6 nucleotides. In some embodiments, the composition comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and (iii) one or more primer nucleic acid molecules having a generate nucleic acid sequence of 6 nucleotides. In some embodiments, the ratio of (i), (ii), and (iii) in the composition is about 8:1:1.

In some embodiments, the RNA molecule is a mature miRNA molecule, and the ratio of (i), (ii), and (iii) in the composition is about 8:1:1.

In some embodiments, the composition comprises (i) one or more primer nucleic acid sequences molecules having a degenerate nucleic acid sequence of 2 nucleotides, (ii) one or more primer nucleic acid molecules having a degenerate nucleic acid sequence of 3 nucleotides, (iii) one or more primer nucleic acid molecules having a generate nucleic acid sequence of 4 nucleotides, and (iv) one or more primer nucleic acid molecules having a generate nucleic acid sequence of 6 nucleotides. In some embodiments, the molar ratio of (i), (ii), (iii), and (iv) in the composition is about 1:1:7:1. In some embodiments, the RNA molecule is an miRNA molecule, an mRNA molecule, or a lncRNA molecule, and the molar ratio of (i), (ii), (iii), and (iv) in the composition is about 1:1:7:1.

In some aspects, provided herein are systems for quantifying ribonucleic acid (RNA) in a sample. In some embodiments, the system comprises a primer nucleic acid molecule, or a mixture of primer nucleic acid molecules. In some embodiments, the primer nucleic acid molecule or each primer nucleic acid molecule comprises a stem-loop structure and a degenerate nucleic acid sequence of 2-10 nucleotides at the 3′ end, wherein the degenerate nucleic acid sequence hybridizes to the 3′-end of an RNA molecule. In some embodiments, the system additionally comprises a reverse transcriptase, and deoxyribonucleotide triphosphates (dNTPs). In some embodiments, the RNA molecule is a mature microRNA (miRNA), a messenger RNA (mRNA), or a long noncoding RNA (lncRNA) molecule. In some embodiments, the degenerate nucleic acid sequence comprises 2, 3, 4, or 6 nucleotides. In some embodiments, the stem comprises 14 base pairs and the loop comprises 6 nucleotides.

In some embodiments, the system comprises a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and/or (iii) one or more primer nucleic acid molecules having a generate nucleic acid sequence of 6 nucleotides. In some embodiments, the system comprises a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and (iii) one or more primer nucleic acid molecules having a generate nucleic acid sequence of 6 nucleotides. In some embodiments, the molar ratio of (i), (ii), and (iii) in the composition is about 8:1:1. In some embodiments, the RNA molecule is a mature miRNA molecule, and the molar ratio of (i), (ii), and (iii) in the composition is about 8:1:1.

In some embodiments, the system comprises a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 3 nucleotides, (iii) one or more primer nucleic acid molecules having a generate nucleic acid sequence of 4 nucleotides, and (iv) one or more primer nucleic acid molecules having a generate nucleic acid sequence of 6 nucleotides. In some embodiments, the molar ratio of (i), (ii), (iii), and (iv) in the composition is about 1:1:7:1. In some embodiments, the RNA molecule is a mature microRNA (miRNA), a messenger RNA (mRNA), or a long noncoding RNA (lncRNA) molecule and the molar ratio of (i), (ii), (iii), and (iv) in the composition is about 1:1:7:1.

In some aspects, provided herein are methods of quantifying micro RNA (miRNA) in a sample. In some embodiments, the method comprises contacting the sample with a system described herein. In some embodiments, the method comprises contacting he sample with a system described herein under conditions whereby the primer nucleic acid molecule or mixture of primer nucleic acid molecules hybridizes to miRNA present in the sample and reverse transcription of the miRNA occurs. In some embodiments, the method comprises amplifying and quantifying the reverse transcribed miRNA using quantitative real-time PCR.

In some aspects, provided herein are methods of quantifying micro RNA (miRNA), long noncoding RNA (lncRNA), and/or messenger RNA (mRNA) in a sample. In some embodiments, the method comprises contacting the sample with a system described herein. In some embodiments, the method comprises contacting the sample with a system comprising a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 3 nucleotides, (iii) one or more primer nucleic acid molecules having a generate nucleic acid sequence of 4 nucleotides, and (iv) one or more primer nucleic acid molecules having a generate nucleic acid sequence of 6 nucleotides. In some embodiments, the molar ratio of (i), (ii), (iii), and (iv) in the composition is about 1:1:7:1. In some embodiments, the method comprises contacting the sample with system under conditions whereby the mixture of primer nucleic acid molecules hybridizes to miRNA, lncRNA, and/or mRNA present in the sample and reverse transcription of the miRNA, lncRNA, and/or mRNA occurs. In some embodiments, the method comprises amplifying and quantifying the reverse transcribed miRNA, lncRNA, and/or mRNA.

In some embodiments, the sample is a biological sample. In some embodiments, the biological sample comprises mammalian cells. In some embodiments, the mammalian cells are human cells.

### BRIEF DESCRIPTION OF THE DRAWING(S)

FIG. 1A and FIG. 1B are schematic representations of the universal hairpin primer (UHP) system. FIG. 1A shows schematics of conventional hairpin (or stem-loop) primer-based qPCR analysis of miRNA expression. A miRNA-specific hairpin primer (MsHP) contains six nucleotides (nt) complementary to the 3′-end of mature miRNA, followed by a stem-loop structure. Once MsHP anneals to the targeted miRNA (a), RT reaction is carried out (b). The resultant RT product is used as a template for real-time quantitative PCR analysis (c) using a forward primer matching to the 5′-end of the mature miRNA, and a reverse primer complementary to the 3′-end of the hairpin or stem-loop structure. FIG. 1B shows the schematic structure and nucleotide sequences of the tested universal hairpin primers (HUPs). MsHP is a representative miRNA-specific hairpin primer that contains a 14-bp stem, 16-nt loop and 6-nt complementary to 3′-end of mature miRNA (indicated as “x”). UHP2, UHP3, UHP4 and UHP6 represent the four universal hairpin primers (UHPs) and share the same hairpin sequence as that of MsHP's, except that they contain 2, 3, 4, and 6 randomized nucleotides at the 3′-end of the stem sequence.

FIG. 2A-FIG. 2C illustrate the sensitivity and specificity of the UHP-based qPCR analysis of miRNA expression in comparison with MsHPs. FIG. 2A & FIG. 2B show dynamic range and standard curve analysis of UHPs vs. MsHP. UHP and MsHP-derived RT products were subjected to 4-fold serial dilutions and used for TqPCR. Three representative miRNAs, HSAMIR-122-5P (a), HSAMIR-181A-5P (b), and HSAMIR-1268A (c), were selected for dynamic range of amplification (FIG. 2A) and melt curve analysis (FIG. 2B). Standard curves are shown in FIG. 6. FIG. 2C shows amplification specificity. The qPCR end-products with expected sizes of ˜65 bp were assessed by electrophoresis on 2% agarose gels. Only the results from the 1:160 dilution groups (the second dilution) for the three miRNAs are shown.

FIG. 3A-FIG. 3C show validation of the tetramer UHP4 as the “winning” universal primer among the tested four UHPs. FIG. 33A shows Cq value comparison of the four UHPs relative to MsHP. RT products prepared with the MsHP and the four UHPs were subjected to qPCR analysis of the indicated 14 miRNA expression. The average Cq values were calculated and plotted. N2=UHP2, N3=UHP3, N4=UHP4, and N6=UHP6. FIG. 3B shows the heatmap and cluster analysis of ΔCq value relative to MsHP for each UHP. The ΔCq value was calculated by subtracting each UHP's average Cq value from respective MsHP's Cq value. 5s RNA was included as an internal reference transcript. FIG. 3C shows a box and whisker plot of ΔCq value relative to MsHP for each UHP. The nonparametric Kruskal-Wallis test was carried out to assess the statistical difference among the four UHPs.

FIG. 4A-FIG. 4C show the effect of large transcripts on miRNA quantification on the UHP-based qPCR system. FIG. 4A shows removal of large transcripts from total RNA using size-selection magnetic beads. Total RNA was mixed with Mag-Bind beads at vol/vol ratio of 1:1 to isolate small RNAs (sRNA, i.e., <200 nt). The purified sRNA was assessed by an Agilent 2100 Bioanalyzer, and the results were visualized in both gel images (a) and electropherograms (b). FIG. 4B shows average Cq values of the 14 tested miRNAs in total RNA vs. purified sRNA samples for RT reactions using MsHP or UHP4. T-MsHP and T-UHP4 indicate the RT products of the total RNA sample prepared with MsHP and UHP4 primers, respectively. P-MsHP and P-UHP4 indicate the RT products of the purified sRNA sample prepared with MsHP and UHP4 primers, respectively. FIG. 4C shows a box and whisker plot, linear regression and correlation coefficient analysis of miRNA detection in total RNA vs. purified sRNA. Linear regression and correlation of the average Cq value correlations between total RNA and purified sRNA samples using MsHP (b) or UHP4 (c) were also analyzed.

FIG. 5A-FIG. 5C illustrate the characterization and identification of the optimized UHP (OUHP) cocktail mixtures as potential MsHP surrogates. FIG. 5A shows compositions of the 15 UHP mixtures of UHP2, UHP4 and UHP6 at various molar percentages, and the UHP4 as a reference control (a). The Cq values of the analyzed 14 miRNAs with the 15 UHP mixtures, along with MsHP (T) and UHP4 (N4) RT products (b). FIG. 5B shows a heatmap analysis of the Cq values of the analyzed 14 miRNAs with the 15 UHP mixtures, along with MsHP (T) and UHP4 groups. The heatmap was generated by using complete linkage clustering method with Spearman Rank Correlation as distance measurement method. MsHP (T) group is boxed, while Mix3 and UHP4 groups are highlighted. FIG. 5C shows heatmap analysis of the ΔCq values of the analyzed 14 miRNAs with the 15 UHP mixtures, along with UHP4 group. The heatmap was generated by using complete linkage clustering method with Spearman Rank Correlation as distance measurement method. UHP4 group is highlighted. The ΔCq value was calculated as the follows: ΔCq=Cq (MsHP)−Cq (UHP Mix).

FIG. 6A and FIG. 6B show standard curve analysis of UHPs. For FIG. 6A, RT products prepared by using MsHP and four UHP primers were 4-fold serially diluted and subjected to TqPCR analysis using specific forward primers for HSA-MIR-122-5P (a), HSA-MIR-181A-5P (b), and HSA-MIR-1268A (c). The standard curves were generated from the obtained Cq values. Linear regression analysis was performed and correlation coefficients were determined. For FIG. 6B, total RNA from HEK-293 cells was 4-fold serially diluted and subjected to reverse transcription using MsHP and four UHP primers, followed by TqPCR analysis using specific forward primers for HSA-MIR-122-5P (a), HSA-MIR-181A-5P (b), and HSA-MIR-1268A (c). Dynamic range of amplification and standard curve analysis were carried out as described in (FIG. 6A).

FIG. 7A-FIG. 7C illustrate the effect of large transcripts on miRNA qPCRs. Total RNA was isolated from HEK293 (FIG. 7A), A375 (FIG. 7B) and 143B (FIG. 7C) cells, and subjected to magnetic bead-based size selection to remove RNA species >200 nt. The resulting RNA samples were designated as P-293, P-A375 and P-143B, as opposed to their control counterparts T-293, T-A375 and T-143B. These RNA samples were subjected to RT reactions using MsHP, followed by qPCR analysis of the five miRNAs. All qPCR reactions were done in triplicate.

FIG. 8A is a list of the tested 15 UHP cocktail mixtures in molar compositions (%). FIG. 8B is a distribution of the ΔCq values for the 15 tested UHP mixtures. Positive ΔCq values indicate lower Cq values in the UHP groups than that in the MsHP group, suggesting overestimation; and vice versa for the negative ΔCq values.

FIG. 9 is a box plot analysis of ΔCq values of the 15 tested UHP mixtures. Positive ΔCq values indicate lower Cq values in the UHP groups than that in the MsHP group, and vice versa for the negative ΔCq values.

FIG. 10A-FIG. 10C illustrate detection specificity analysis of the LET7 family. For FIG. 10A, total RNA was subjected to RT reactions using the individual LET7-specific HP (LET7-specific), the pooled LET7 family-specific HP (LET7 FSP), or OUHP, followed by qPCR analysis with LET7-specific forward primers. FIG. 10B shows dynamic amplification range and standard curve analyses. Ten nanograms (ng) of the synthetic mature miRNAs LET7d (a) and miR LET7i (b) were subjected to 4-fold serial dilutions, followed by RT reactions using OUHP and qPCR analysis with forward primers for LET7d (a) and LET7i (b), respectively. FIG. 10C shows specificity of detection analysis. The synthetic mature miRNAs LET7e (a), LET7g (b), and LET7i (c) (about 100 ng) were subjected to RT reaction with OUHP and qPCR analysis with LET-specific forward primers. “**” p<0.01, compared with the average Cq value obtained with the forward primer for the respective synthetic LET7 mature miRNA.

FIG. 11A-11B show a schematic representation of the degenerate hairpin primer (DHP) system and its mode of action. FIG. 11A is a schematic showing the degenerate hairpin primers (DHPs) (or stem-loop) contain a 14-bp stem and 16-nt loop, followed by 2, 3, 4, 5, and 6 randomized nucleotides at the 3′-end of the stem sequence (a), designated as DHP2, DHP3, DHP4, DHP5, and DHP6 (b). As positive controls, transcript-specific hairpin primers (TSPs) contain 18 nucleotides (nt) complementary to respective transcripts, proceeded with the same stem-loop structure. FIG. 11B is a schematic showing Universal TaqMan (U-Taqman) probe-based qPCR. The coding or noncoding transcripts of interest are denatured and annealed with DHPs, TSPs primers, or the machine learning-optimized Cocktail of Degenerate hairpin primers (such as COD24), and subjected to reverse transcription (RT) (a). The resultant RT products are diluted and used for universal TaqMan (U-TaqMan)-based qPCR reactions with transcript-specific (TS) forward primers and a common reverse primer. The DHP sequence is fully incorporated into the qPCR products after the first cycle (b). Starting the second cycle, the U-TaqMan probe hybridizes to the first round PCR products, and the forward primer-initiated extension driven by Taq DNA polymerase leads to the removal of the fluorescent dye from U-TaqMan probe, which is catalyzed by the 5′-exonuclease activity of Taq DNA polymerase (b). The freed dye signals are proportional to the expression levels of transcripts.

FIG. 12A-FIG. 12B show sensitivity and specificity of the DHP-based qPCR analysis of mRNA expression in comparison with TSPs. FIG. 12A-FIG. 12B show dynamic range analysis of DHPs vs. TSP. Total RNA (2 μg) was isolated from HEK-293 cells and subjected to RT reactions with DHPs and TSPs. The resultant RT products were subjected to 4-fold serial dilutions and used for TqPCR. Three representative mRNAs, HNRNPL (a), RER1 (b), and HMBS (c), were analyzed for dynamic range of amplification (FIG. 12A). The corresponding standard curves are shown in FIG. 17. FIG. 12B shows ΔCq comparison analysis of the four genes. The ΔCq values were calculated by subtracting the Cq (TSP) from the Cq (DHPs). The obtained ΔCq values for the 4 tested genes were subjected to heatmap and clustering analysis (a) and box and whisker plot analysis (b). (C) ΔCq comparison analysis of the 37 tester genes. The calculated ΔCq values for the 37 genes were subjected to heatmap analysis (a) and box and whisker plot analysis (b). A detailed heatmap and clustering analysis results are listed in FIG. 18. In the heatmaps, negative ΔCq values (red color) indicate potential overestimation of expressions, whereas positive ΔCq values (green color) imply the opposite (underestimation). The heatmap was generated by using complete linkage clustering method with Spearman Rank Correlation as distance measurement method.

FIG. 13A-FIG. 13C shows identification of the optimal Cocktails of DHPs (CODs) as potential TSP surrogates through linear regression-based machine learning analysis. FIG. 13A shows heatmap and clustering analysis of the 24 modeled CODs in % of molar composition. The detailed molar compositions of the 24 CODs are listed in Table 5. FIG. 13B shows Cq correlation between the TSP and the COD groups through machine learning linear regression analysis. Total RNA were isolated from HEK-293 cells and subjected to RT reactions with either TSP or different CODs. TaqMan qPCR analysis was carried out for the tester genes listed in Table 2. The predicated Cq values for CODs (red line) and TSP (blue line) were calculated and compared through machine learning linear regression analysis. Four representative Cq correlation curves are shown, while the correlation curves for the rest CODs are shown in FIG. 19. The detailed coefficient of correlation, slope value, intercept value, and p-value for Paired-Samples T Test (using python SciPy package) for the 24 CODs are listed in Table 6. FIG. 13C shows a box and whisker plot analysis of ΔCq value relative to TSP for each COD. The nonparametric Kruskal-Wallis test was carried out to assess the statistical difference among the 24 CODs. The dotted line represents the ΔCq value of zero.

FIG. 14A-FIG. 13C shows validation and characterization of the COD24 as an optimal TSP surrogate. FIG. 14A shows COD24 as a reliable TSP surrogate in RT-qPCR-based quantification of expression. Total RNA was isolated from 143B (a), Mel-624 (b), Mel-888 (c), A375 (d), SJSA1 (e), and UC-MSC (f) cells, and subjected to RT reactions using the TSP and COD24 primers, followed by TaqMan qPCR analysis of the 8 selected genes. FIG. 14B shows the effect of forward primer locations on TaqMan qPCR quantification of expression. Total RNA was isolated from HEK-293 cells and subjected to RT reactions with the COD24 primers. Multiple forward primers with different locations for AXIN2 (a) and RUNX2 (b) transcripts were used for TaqMan qPCR analysis. The nonparametric Kruskal-Wallis test was carried out to assess the statistical difference among the Cq values yielded by the forward primers with different locations as indicated at the x-axis. FIG. 14C shows a comparison of expression quantification between the COD24/TaqMan and conventional hexamer/SYBR Green systems. Subconfluent UC-MSC cells were infected with adenoviral vectors Ad-GFP, Ad-Wnt1 or Ad-Wnt3 (a). At 48 h after infection, total RNA was isolated from the infected cells and subjected to RT reactions with the COD24 primers or the conventional hexamer. The RT products derived from COD24 were used for qPCR analysis of the Wnt target gene expression with either TaqMan probe/common reverse primer (b) or SYBR Green/gene-specific reverse primers (c). The RT products derived from conventional hexamer were used for qPCR analysis of the expression of the same set of Wnt target genes with SYBR Green/gene-specific primers (d). GAPDH was used as a reference gene. “*” p<0.05; “**” p<0.01, when compared with that of the Ad-GFP control group.

FIG. 15A-FIG. 15D shows COD24-based RT-qPCR expression quantification of lncRNAs and miRNAs with high specificity. FIG. 15A shows COD24-based TaqMan qPCR quantification of lncRNA expression. Total RNA was isolated from HEK-293 cells, and subjected to RT reactions with COD24 primers or lncRNA-specific primers (TSPs), followed by TaqMan qPCR analysis of the 10 selected lncRNAs (Table 7). No statistical significance in the Cq values between COD24 and TSP groups was found for any of the tested lncRNAs. FIG. 15B shows COD24-based TaqMan qPCR quantification of miRNA expression. Total RNA was isolated from HEK-293 cells, and subjected to RT reactions with COD24 primers or miRNA-specific primers (TSPs), followed by TaqMan qPCR analysis of the 14 miRNAs (Table 7). No statistical significance in the Cq values between COD24 and TSP groups was found for any of the tested miRNAs. FIG. 15C shows dynamic range of amplification and standard curves for COD24-based TaqMan qPCR quantification of miRNA LET-7 isomiR family members. 10 ng of synthetic mature miRNA LET-7e (a), LET7g (b) and LET-7i (c) were subjected to RT reactions with COD24 primers. The RT products were 4-fold serially diluted and used for TaqMan qPCR analysis of LET-7e (a), LET7g (b) and LET-7i (c) expression using respective forward primers. FIG. 15D shows COD24-based TaqMan qPCR quantification of miRNA LET-7 isomiRs with high specificity. The mature miRNA sequences and isomiR-specific forward primers for the eight LET-7 members are shown (a). 10 ng of synthetic mature miRNA LET-7e (b) and LET-7i (c) were subjected to RT reactions with COD24 primers. The RT products were used for TaqMan qPCR analysis of LET-7 isomiR expression using respective forward primers. “**” p<0.01, when compared with that of LET-7e (b) or LET-7i (c).

FIG. 16A-FIG. 16C shows average transcript abundances of human transcriptome. FIG. 16C shows the CCLE dataset: RNA-seq data (in Reads Per Kilobase of transcript per Million mapped reads, or RPKM) of 1,076 human cancer cells were reported in the Broad Institute Cancer Cell Line Encyclopedia (CCLE; https://portals.broadinstitute.org/ccle) was downloaded from the UCSC XENA datasets (https://xenabrowser.net/datapages/). FIG. 16B shows the GTEx dataset: RNA-seq data (in Transcripts per million, or TPM) of 54 types of normal human tissues were downloaded from The Genotype-Tissue Expression (GTEx) project (https://gtexportal.org/home/). FIG. 16C shows the CA-14 dataset: RNA-seq data (in Transcripts per million, or TPM) of 14 human cancer lines (including osteosarcoma, melanoma and GI cancer lines) were generated from an RNA-seq dataset developed in other studies.

FIG. 17 shows qPCR amplification standard curves for DHP and TSP-based RT-PCR products. TaqMan-based qPCR analysis was carried out on the RT-PCR products derived from DHP2, DHP3, DHP4, DHP5, DHP6, and TSP-initiated reverse transcription reactions (FIG. 12A). The representative results from the three genes HNRNPL (a), RER1 (b), and HMBS (c) are shown.

FIG. 18 shows a detailed heatmap of the ΔCq values for the 37 tester genes from DHP and TSP-based RT-PCR products. The ΔCq values were calculated as: ΔCq=Cq (DHP) −Cq (corresponding TSP). Thus, negative ΔCq values (red color) indicate potential overestimation of expressions, whereas positive ΔCq values (green color) imply the opposite (underestimation).

FIG. 19 shows Cq correlation between the TSP and the COD groups through linear regression-based machine learning analysis. The molar compositions of the 24 Cocktails of Degenerate hairpins (CODs) are listed in Table 5. Total RNA were isolated from HEK-293 cells and subjected to RT reactions with either TSP or different CODs. TaqMan qPCR analysis was carried out for varied numbers of the tester genes listed in Table 2. The predicated Cq values for CODs (red line) and TSP (blue line) were calculated and compared through machine learning linear regression analysis. The Cq correlation curves for 20 of the 24 CODs are listed here while the correlation curves for COD10, COD21, COD23 and COD24 are listed in FIG. 13B. The detailed coefficient of correlation, slope value, intercept value, and p-value for Paired-Samples T Test (using python SciPy package) are listed in Table 6.

### DETAILED DESCRIPTION

The present disclosure is predicated, at least in part, on the development of a cost-effective and reliable universal hairpin primer (UHP) system that not only obviates the need for RNA-specific primers in reverse transcription reactions (e.g. miRNA-specific hairpin primers (MsHPs)) but also has high throughput potential. The term “universal hairpin primer” and “degenerate hairpin primer” are used interchangeably herein and refer to primers described herein that comprise degenerate nucleic acids at the 3′ end. For example, a panel of four universal hairpin primers (UHPs) were analyzed that share the same step-loop hairpin structure but are anchored with 2, 3, 4 and 6 degenerate nucleotides at their 3′-ends (namely, UHP2, 3, 4 and 6). All four degenerate UHPs yielded robust RT products and specifically quantified individual miRNAs by qPCR with high efficiency similar to that of MsHPs. As described herein, the UHP-based RT-qPCR miRNA quantification is not affected by the presence of ribosomal RNAs and long transcripts. The universal hairpin primers described herein can serve as a surrogate for any miRNA-specific hairpin primer for real-time quantitative PCR-(RT-aPCR) based quantification of miRNA expression in a cost-effective and/or high throughput fashion, and are valuable tools for basic research and precision medicine. In addition, the system described herein may be readily adapted for other forms of qPCR detection chemistry, such as TaqMan, cycling probe technology (CPT), molecular beacons, and minor groove binding (MGB) probes. Moreover, the disclosed primers may be adapted for use in multiplex analysis of miRNA expression. The primers described herein are also shown to be effective for quantification of both coding and noncoding RNAs. Through comparison with transcript-specific hairpin primers (TSPs) on 37 tester genes and linear regression-based machine learning analysis of 24 cocktails of DHPs (CODs), an optimal DHP mix (i.e., COD24) was identified, which best recapitulated the TSPs in mRNA quantification. As described herein, the COD24-mediated U-TaqMan qPCR system effectively quantified the expression levels of lncRNAs and miRNAs with high sensitivity and specificity. This system provides a cost-effective tool for coding and noncoding transcriptomic quantification, has broad applications in basic and translational research, as well as in clinical diagnostics.

### Definitions

To facilitate an understanding of the present technology, a number of terms and phrases are defined below. Additional definitions are set forth throughout the detailed description.

Nomenclature for nucleotides, nucleic acids, nucleosides, and amino acids used herein is consistent with International Union of Pure and Applied Chemistry (IUPAC) standards (see, e.g., bioinformatics.org/sms/iupac.html).

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, uracil, adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

When referring to a nucleic acid sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math., 2: 482 (1981), by the sequence identity alignment algorithm of Needleman & Wunsch, J Mol. Biol., 48,443 (1970), by the search for similarity method of Pearson & Lipman, Proc. Natl. Acad. Sci. USA, 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res., 12, 387-395 (1984), or by inspection. Another algorithm is the BLAST algorithm, described in Altschul et al., J Mol. Biol., 215, 403-410, (1990) and Karlin et al., Proc. Natl. Acad. Sci. USA, 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res., 25, 3389-3402. Unless otherwise indicated, percent identity is determined herein using the algorithm available at the internet address: blast.ncbi.nlm.nih.gov/Blast.cgi.

The terms “coding sequence,” “coding sequence region,” “coding region,” and “CDS,” when referring to nucleic acid sequences, may be used to refer to the portion of a DNA or RNA sequence, for example, that is or may be translated to protein. In contrast, a “noncoding” or a “non-coding” sequence (e.g. a noncoding RNA) refers to a portion of a sequence that is not translated into a protein. For example, a noncoding RNA refers to an RNA sequence that is not translated into a protein. The terms “reading frame,” “open reading frame,” and “ORF,” may be used herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The terms “complementary” and “complementarity” refers to the relationship between two nucleic acid sequences or nucleic acid monomers having the capacity to form hydrogen bond(s) with one another by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., about 50%, about 60%, about 70%, about 80%, about 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleic acid sequences hybridize under at least moderate, or, in some embodiments high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (optionally in combination with EDTA). Additional details and an explanation of stringency of hybridization reactions are provided in, e.g., Sambrook, supra; and Ausubel et al., eds., Short Protocols in Molecular Biology, 5th ed., John Wiley & Sons, Inc., Hoboken, N.J. (2002). The term “hybridization” or “hybridized” when referring to nucleic acid sequences is the association formed between and/or among sequences having complementarity.

The terms “primer,” “primer sequence,” and “primer oligonucleotide,” as used herein, refer to an oligonucleotide which is capable of acting as a point of initiation of synthesis of a primer extension product that is a complementary strand of nucleic acid (all types of DNA or RNA), when placed under suitable amplification conditions (e.g., buffer, salt, temperature and pH) in the presence of nucleotides and an agent for nucleic acid polymerization (e.g., a DNA-dependent or RNA-dependent polymerase). A primer can be single-stranded or double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. A “forward primer” is a primer that hybridizes (or anneals) to a target nucleic acid sequence (e.g., template strand) for amplification. A “reverse primer” is a primer that hybridizes (or anneals) to the complementary strand of the target sequence during amplification. A forward primer hybridizes with a target sequence 5′ with respect to a reverse primer.

The term “secondary structure,” or “secondary structure element,” or “secondary structure sequence region” as used herein in reference to nucleic acid sequences (e.g., RNA, DNA, etc.), refers to any non-linear conformation of nucleotide or ribonucleotide units. Such non-linear conformations may include base-pairing interactions within a single nucleic acid polymer or between two polymers. Single-stranded RNA typically forms complex and intricate base-pairing interactions due to its increased ability to form hydrogen bonds stemming from the extra hydroxyl group in the ribose sugar. Examples of secondary structures or secondary structure elements include but are not limited to, for example, stem-loops, hairpin structures, bulges, internal loops, multiloops, coils, random coils, helices, partial helices and pseudoknots. In some embodiments, the term “secondary structure” may refer to a stem-loop structured RNA element (SuRE) element.

The term “recombinant,” as used herein, means that a particular nucleic acid (DNA or RNA) is the product of various combinations of cloning, restriction, polymerase chain reaction (PCR) and/or ligation steps resulting in a construct having a structural coding or noncoding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, the artificial combination may be performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may comprise a naturally occurring amino acid sequence.

The terms “microRNA (miRNA)” and “mature miRNA” are used interchangeably herein and refer to small (approximately 18-24 nucleotides in length), noncoding RNA molecules present in the genomes of plants and animals. In certain instances, highly conserved, endogenously expressed miRNAs regulate the expression of genes by binding to the 3′-untranslated regions (3′-UTR) of specific mRNAs. More than 1000 different miRNAs have been identified in plants and animals. Certain mature miRNAs appear to originate from long endogenous primary miRNA transcripts (also known as pri-miRNAs, pri-mirs, pri-miRs or pri-pre-miRNAs) that are often hundreds of nucleotides in length (Lee, et al., EMBO J., 21(17): 4663-4670 (2002)).

The terms “long noncoding RNA”, “long non-coding RNA”, or “lncRNA” are used interchangeably herein and refer to a noncoding RNA molecule of more than 200 nucleotides in length.

The terms “messenger RNA” or “mRNA” are used interchangeably herein and refer to a single-stranded RNA made from a DNA template during transcription. mRNA is an example of coding RNA which is translated into a protein.

### Primers

Provided herein are primers. In some embodiments, provided herein are primer nucleic acid sequences for quantifying RNA in a sample. The primer nucleic acid sequences described herein comprise a stem-loop structure and a degenerate nucleic acid sequence of 2-10 nucleotides at the 3′ end, wherein the degenerate nucleic acid sequence hybridizes to the 3′-end of the RNA molecule. The primers described herein can bind to and thus can be used to quantify coding and/or noncoding RNAs. In some embodiments, the degenerate nucleic acid sequence hybridizes to the 3′ end of a coding RNA molecule. In some embodiments, the degenerate nucleic acid sequence hybridizes to the 3′ end of a noncoding RNA molecule. In some embodiments, the RNA molecule is a microRNA (miRNA), a messenger RNA (mRNA), or a long noncoding RNA (lncRNA) molecule.

In some aspects, provided herein are primer nucleic acid sequences for quantifying miRNA in a sample. The increasing recognition of miRNAs' biological functions in regulating many aspects of cellular processes mandates readily available technologies to quantify miRNA expression. Numerous techniques have been developed to assess miRNA expression levels (9-12). The conventional Northern blotting (NB) technique was first used for the initial discovery of miRNA lin-4 in 1993 (1), and remains the only technique that allows for the quantitative visualization of miR (9). The NB technique was later modified by labeling DNA probes with 3′-digoxigenin (DIG) hapten to avoid the use of radioisotopes, and/or by using locked nucleic acid (LNA) in nucleic acid probes to improve sensitivity and match specificity (9). However, compared with other detection methods, NB suffers from low sensitivity, is time-consuming, low throughput, and has requires a large quantity of RNA. Similar to NB, miRNA microarray analysis relies on the sensitive, specific hybridization of the target miR to its complementary DNA probe, which is spatially organized on a solid phase or gene chip, and visualized with fluorescence or imaging instrumentation. Microarray analysis of miRNA expression represents one of the earliest techniques capable of high throughput and massive parallel analysis of numerous miRNAs in one sample at the same time. Drawbacks of the microarray method include relative higher cost, limited dynamic range of detection, semi-quantitative nature of detection, secondary validation requirement, and limited specificity on closely related miRNA sequences.

In recent years, next-generation sequencing (NGS) has become a viable technique to quantify miRNA expression (9-12, 28). Other emerging detection methods include various biosensor techniques involved in electrochemical-based detection, optical-based detection, and nanotube-based methodology, and nucleic acid amplification techniques such as rolling circle amplification (RCA), duplex-specific nuclease (DSN)-based amplification, loop-mediated isothermal amplification (LAMP), exponential amplification reaction (EXPAR), and strand-displacement amplification (SDA) (9-12). Each of these detection techniques has its unique advantages as well as inherent shortcomings including long processing times, laborious procedures, low throughput, large sample size requirements, false positives, lack of sensitivity, and/or costly instrument requirements.

Given the advantages in detection sensitivity, high throughput potential, and technical ease, quantitative or real-time RT-PCR (RT-qPCR) analysis has become the most popular method to detect and quantify miRNA expression (9-12, 29). RT-qPCR is based on reverse transcription of RNA to cDNA, followed by a quantitative polymerase chain reaction. The accumulation of the reaction product is followed in real time at each cycle of PCR. The first use of a qPCR-based method for miRNA quantification was described in 2004, in which two forward primers and one reverse primer were used to detect the expression of pri- and pre-miRNA levels (30).

Numerous efforts have been devoted to increasing miRNA length at the RT stage, primarily focusing on two approaches: poly(A) tailing and the use of stem-loop/hairpin adaptor/primers (9-12, 29). The former approach involves the use of poly(A) polymerase-mediated polyadenylation, a poly(T) adapter, and a miRNA-specific forward primer (15). A variation of the poly(A) tailing approach involves the use of T4 RNA ligase to uniformly extend microRNAs' 3′-ends by adding a linker-adapter, which then serves as an ‘anchor’ to prime cDNA synthesis and throughout qPCR to amplify specific target amplicons (31). The use of stem-loop or hairpin primers for miRNA RT reactions followed by TaqMan PCR analysis was also introduced in 2005 (14), although several modifications, including the use of a universal TaqMan probe and longer stem-loop RT primers, have been reported (32,33). A recently reported stem-loop variation called Dumbbell-PCR method takes advantage of the T4 RNA ligase 2-mediated ligation of either 5′- or 3′-end stem-loop adapter to target miRNAs (34). While most of these RT-qPCR based methods provide high sensitivity and specificity for miRNA quantification, these systems require the use of miRNA-specific hairpin primers, which is not cost-effective, time-consuming, and/or has low throughput.

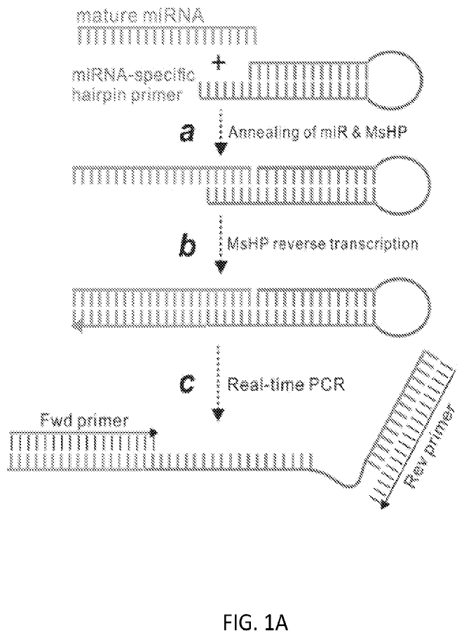

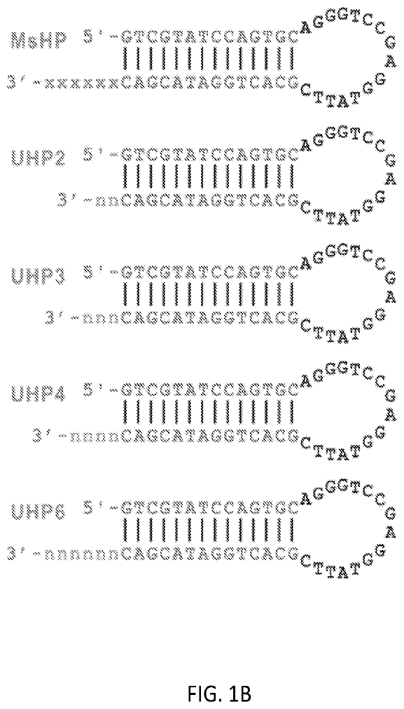

The conventional miRNA-specific stem-loop (or hairpin) primer-based RT-PCR method is widely used to quantify miRNA expression (14). In this system, a miRNA-specific hairpin primer (MsHP) contains six nucleotides (nt) complementary to the 3′-end of mature miRNA, followed by a stem-loop structure, as shown in FIG. 1A. Once a MsHP anneals to the targeted miRNA, RT reaction is carried out and the resultant RT product is used as a template for real-time quantitative PCR analysis using a forward primer complementary to the 5′-end of the mature miRNA, and a reverse primer complementary to the 3′-end of the hairpin or stem-loop structure (FIG. 1A). While the MsHP system for quantifying miRNA is robust, it is not cost-effective for large scale and/or high throughput analysis of multiple miRNAs simultaneously.

Thus, the present disclosure provides primer nucleic acid sequences for quantifying mature miRNA in samples.

In some aspects, provided herein are primer nucleic acid sequences that can bind to miRNA, mRNA, or lncRNA. The classic central dogma of molecular biology states the coded genetic information hard-wired into DNA is transcribed into individual transportable cassettes composed of messenger RNA (mRNA), each of which contains the program for synthesis of a particular protein or small number of proteins to carry out cellular functions (1-3). While the central dogma flow of genetic information from DNA to RNA to protein has been held true in general, there have been some well-known exceptions: retroviruses transcribe RNA into DNA through a specialized enzyme reverse transcriptase (RT) resulting in RNA to DNA to RNA to protein; some primitive viruses only use RNA to proteins; certain RNA molecules called ribozymes possess enzymatic functions; and prion proteins directly replicate themselves and thus enable the information flow from proteins to the genome. Furthermore, the rapid progresses in genome biology in the past three decades have revealed that, while less than 2% of human genome is utilized to make proteins through coding RNAs, approximately 70% of human genome has been transcribed, most of which is categorically called noncoding RNAs (ncRNAs), including small interfering RNA (siRNA), microRNA (miRNA), and long noncoding RNA (lncRNA) (1, 2, 4, 5). It has been shown that the amount of noncoding genome increases with organism complexity, ranging from 0.25% of prokaryotes' genome to 98.8% of humans (2, 5, 6). Increasing evidence suggests that ncRNAs may play important regulatory roles in cellular processes as well as in pathological processes (1-3). Accordingly, quantification of both coding and noncoding RNA is an important goal that is achieved using the primers, methods, and systems described herein.

In some embodiments, the primer nucleic acid molecule comprises a stem-loop structure and a degenerate nucleic acid sequence of 2-10 (e.g., 2-6) nucleotides at the 3′ end, wherein the degenerate nucleic acid sequence hybridizes to 3′-end of an RNA molecule. In some embodiments, the RNA molecule comprises mature micro RNA (miRNA) molecule. In some embodiments, the RNA molecule comprises a miRNA, mRNA, or lncRNA molecule. This primer nucleic acid molecule is also referred to herein as a “universal hairpin primer” (UHP). The primer nucleic acid molecule may comprise any suitable nucleotide sequence that forms a stem-loop structure of any suitable size. For example, the stem may comprise 10-20 base pairs (e.g., 11, 12, 13, 14, 15, 16, 17, 18, or 19 base pairs), while the loop may comprise 10-20 nucleotides (e.g. 11, 12, 13, 14, 15, 16, 17, 18, or 19 nucleotides). In some embodiments, the stem comprises 14 base pairs and the loop comprises 16 nucleotides. In some embodiments, the sequence forming the stem-loop structure is the same sequence that forms a stem-loop structure in an miRNA-specific, an mRNA specific, or a lncRNA specific hairpin primer. Such nucleic acid sequences include, but are not limited to, 5′-GTC GTA TCC AGT GCA GGG TCC GAG GTA TTC GCA CTG GAT ACG AC-3′ (SEQ ID NO: 1) (see, e.g., Kramer, M. F., Curr Protoc Mol Biol., CHAPTER: Unit 15.10. (2011) doi: 10.1002/0471142727.mb1510s95; and Chen et al., Nucleic Acids Res., 33(20): e179 (2005)). Other stem-loop primers for quantification of miRNAs, and methods for synthesizing such primers, are described in, e.g., Mohammadi-Yeganeh et al., Mol Biol Rep., 40(5): 3665-74 (2013); and Yang et al., PLoS ONE, 9: e115293 (2014).

Instead of nucleotides (nt) that are complementary to the 3′-end of the RNA molecule (e.g. the mature miRNA, the mRNA, the lncRNA) as are found in a RNA-specific hairpin primer (e.g. an MsHP), the primer nucleic acid molecules of the present disclosure comprise a degenerate nucleic acid sequence at the 3′ of the stem sequence. A “degenerate nucleic acid sequence” is one in which one or more nucleotides can perform the same function or yield the same output as a structurally different nucleotide. In other words, in a degenerate nucleic acid sequence multiple different nucleotides are possible at a particular position. In some embodiments, the “degenerate” nucleic acid sequence is also referred to herein as a “randomized” nucleic acid sequence. The degenerate nucleic acid sequence may be any suitable sequence of any length, so long as the degenerate nucleic acid sequence hybridizes to the to 3′-end of an RNA molecule to facilitate reverse transcription of the RNA. For example, the nucleic acid sequence may be any suitable sequence of any length, so long as the degenerate nucleic acid sequence hybridizes to the 3′ end of a mature miRNA molecule, the 3′ end of an mRNA molecule, or the 3′ end of a lncRNA molecule to facilitate reverse transcription. The degenerate nucleic acid sequence desirably comprises 2-10 nucleotides (e.g., 3, 4, 5, 6, 7, 8, or 9 nucleotides).

Exemplary primer nucleic acid sequences are set forth in Table 1; however, the disclosure is not limited to these particular sequences. Additional exemplary primer nucleic acid sequences are set forth in Table 3; however the disclosure is also not limited to these particular sequences. In some embodiments, the primer nucleic acid molecule comprises a degenerate nucleic acid sequence of 4 nucleotides at the 3′ end. In some embodiments, the primer nucleic acid molecule comprises a degenerate nucleic acid sequence of 6 nucleotides at the 3′ end. In some embodiments, the primer nucleic acid molecule comprises a degenerate nucleic acid sequence of 3 nucleotides at the 3′ end. In some embodiments, the primer nucleic acid molecule comprises a degenerate nucleic acid sequence of 2 nucleotides at the 3′ end. Methods and systems for designing and generating primer nucleic acid sequences are known in the art and can be used in the context of the present disclosure. Such systems include online tools such as, e.g., Primer3Plus, Primer-BLAST, and the GenScript Online PCR Primers Designs Tool.

In some embodiments, the primer nucleic acid sequence comprises the sequence of SEQ ID NO: 1 and additionally comprises a degenerate nucleic acid sequence comprising 2-10 nucleotides at the 3′ end of SEQ ID NO: 1. In some embodiments, the primer nucleic acid sequence comprises the sequence of SEQ ID NO: 1 and additionally comprises a degenerate nucleic acid sequence comprising 2, 3, 4, or 6 nucleotides at the 3′ end of SEQ ID NO: 1. Such exemplary primer nucleic acid sequences are included in Table 1, where nucleotides in bold equate to SEQ ID NO: 1, and the non-bold nucleotides at the 3′ end are exemplary degenerate nucleic acid sequences. Any degenerate nucleic acid sequences set forth in Table 1 can be used in the primer nucleic acid sequences, systems, and methods described herein. The sequences shown in Table 1 highlight exemplary sequences having 6 degenerate nucleotides at the 3′ end, but as described above 2-10 degenerate nucleic acids may be used.

TABLE 1

| HAS-MIR | Sequence | SEQ ID NO: | Use |
|---|---|---|---|
| hsa-miR-122-5p | GTCGTATCCAGTGCAGGGTCCGAGG | 2 | RT |
| | TATTCGCACTGGATACGACCAAACA | | |
| hsa-miR-4510 | GTCGTATCCAGTGCAGGGTCCGAGG | 3 | |
| | TATTCGCACTGGATACGACAACCAT | | |
| hsa-miR-192-3p | GTCGTATCCAGTGCAGGGTCCGAGG | 4 | |
| | TATTCGCACTGGATACGACCTGTGA | | |
| hsa-miR-182-5p | GTCGTATCCAGTGCAGGGTCCGAGG | 5 | |
| | TATTCGCACTGGATACGACAGTGTG | | |
| hsa-miR-221-5p | GTCGTATCCAGTGCAGGGTCCGAGG | 6 | |
| | TATTCGCACTGGATACGACAAATCT | | |
| hsa-miR-215-3p | GTCGTATCCAGTGCAGGGTCCGAGG | 7 | |
| | TATTCGCACTGGATACGACTATTGG | | |
| hsa-miR-510-5p | GTCGTATCCAGTGCAGGGTCCGAGG | 8 | |
| | TATTCGCACTGGATACGACGTGATT | | |
| hsa-miR-4425 | GTCGTATCCAGTGCAGGGTCCGAGG | 9 | |
| | TATTCGCACTGGATACGACATGGTC | | |
| hsa-miR-4672 | GTCGTATCCAGTGCAGGGTCCGAGG | 10 | |
| | TATTCGCACTGGATACGACTGCCTC | | |
| hsa-miR-3688-3p | GTCGTATCCAGTGCAGGGTCCGAGG | 11 | |
| | TATTCGCACTGGATACGACAGAGTG | | |
| hsa-miR-151a-5p | GTCGTATCCAGTGCAGGGTCCGAGG | 12 | |
| | TATTCGCACTGGATACGACACTAGA | | |
| hsa-miR-3688-5p | GTCGTATCCAGTGCAGGGTCCGAGG | 13 | |
| | TATTCGCACTGGATACGACATATGG | | |
| hsa-miR-181a-5p | GTCGTATCCAGTGCAGGGTCCGAGG | 14 | |
| | TATTCGCACTGGATACGACACTCAC | | |
| hsa-miR-1268a | GTCGTATCCAGTGCAGGGTCCGAGG | 15 | |
| | TATTCGCACTGGATACGACCCCCCA | | |
| hsa-miR-122-5p | AGCCTGGAGTGTGACAATGGT | 16 | qPCR forward |
| hsa-miR-4510 | AGCCTGAGGGAGTAGGATGTA | 17 | primer |
| hsa-miR-192-3p | AGCCCTGCCAATTCCATAGGT | 18 | |
| hsa-miR-182-5p | AGCCTTTGGCAATGGTAGAAC | 19 | |
| hsa-miR-221-5p | AGCCACCTGGCATACAATGTA | 20 | |
| hsa-miR-215-3p | AGCCTCTGTCATTTCTTTAGG | 21 | |
| hsa-miR-510-5p | AGCCTACTCAGGAGAGTGGCA | 22 | |
| hsa-miR-4425 | AGCCTGTTGGGATTCAGCAGG | 23 | |
| hsa-miR-4672 | AGCCTTACACAGCTGGACAGA | 24 | |
| hsa-miR-3688-3p | AGCCTATGGAAAGACTTTGCC | 25 | |
| hsa-miR-151a-5p | AGCCTCGAGGAGCTCACAGTC | 26 | |
| hsa-miR-3688-5p | AGCCAGTGGCAAAGTCTTTCC | 27 | |
| hsa-miR-181a-5p | AGCCAACATTCAACGCTGTCG | 28 | |
| hsa-miR-1268a | AGCCCGGGCGTGGTGGTGGGG | 29 | |

### Compositions, Systems, and Methods

The disclosure further provides a composition comprising a mixture of two or more of the above-described primer nucleic acid molecules and a carrier. In some embodiments, the composition comprises a mixture of primer nucleic acid sequences that have degenerative nucleic acid sequences of multiple sizes (e.g., 2, 3, 4, and/or 6 nucleotides). For example, the composition may comprise (i) one or more primer nucleic acid molecules having a degenerate nucleic acid sequence of 2 nucleotides (also referred to interchangeably herein as “UHP2” or “DHP2”), (ii) one or more primer nucleic acid molecules having a degenerate nucleic acid sequence of 3 nucleotides (also referred to as “UHP3” or “DHP3”, (iii) one or more primer nucleic acid molecules having a degenerate nucleic acid sequence of 4 nucleotides (also referred to as “UHP4” or “DHP4”), and/or (iv) one or more primer nucleic acid molecules having a generate nucleic acid sequence of 6 nucleotides (also referred to as “UHP6” or “DHP6”). When the mixture is composed of primer nucleic acid sequences with degenerative nucleic acid sequences of different sizes, the primers may be included in the composition in any suitable amount or ratio relative to the degenerative nucleic acid size.

In some embodiments, the composition comprises UHP2, UHP4, and/or UHP6. For example, the composition may be comprised of equal amounts of UHP2, UHP4, and/or UHP6 (i.e., a 1:1:1 ratio). Other ratios of UHP2:UHP4:UHP6 that are encompassed by the present disclosure include, but are not limited to, 1:1:2, 1:2:1, 2:1:1, 1:1:3, 1:3:1, 3:1:1, 1:1:4, 1:4:1, 4:1:1, 1:1:5, 1:5:1, 5:1:1, 1:1:6, 1:6:1, 6:1:1, 1:1:7, 1:7:1, 7:1:1, 1:1:8, 1:8:1, 8:1:1, 1:1:9, 1:9:1, 9:1:1, 1:1:10, 1:10:1, 10:1:1, 1:1:15, 1:15:1, 15:1:1, 1:1:20, 1:20:1, and 20:1:1. In some embodiments, the composition comprises an 8:1:1 mole ratio of UHP2:UHP4:UHP6.

As another example, the composition may comprise DHP2 (i.e. UHP2), DHP3 (i.e. UHP3), DHP4 (i.e. UHP4), and DHP6 (i.e. UHP6). In some embodiments, the composition comprises DHP2:DHP3:DHP4:UHP6 at a 1:1:7:1 molar ratio.

The carrier desirably is a physiologically (e.g., pharmaceutically) acceptable carrier. Any suitable carrier can be used within the context of the disclosure, and such carriers are well known in the art. The choice of carrier will be determined, in part, by the particular use of the composition. In some embodiments, the pharmaceutical composition can be sterile.

The disclosure further provides a system for quantifying RNA in a sample. In some embodiments, provided herein is a system for quantifying miRNA, lncRNA, and/or mRNA in a sample. For example, in some embodiments provided herein is a system for quantifying micro RNA (miRNA) in a sample. In some embodiments, the system comprises (a) a primer nucleic acid molecule, or a mixture of primer nucleic acid molecules, wherein each primer nucleic acid molecule comprises a stem-loop structure and a degenerate nucleic acid sequence of 2-10 (e.g., 2-6) nucleotides at the 3′ end, wherein the degenerate nucleic acid sequence hybridizes to the 3′-end of an RNA molecule as described above (e.g. a mature micro RNA (miRNA) molecule, a lncRNA molecule, or an mRNA molecule); (b) a reverse transcriptase (RT); and (c) deoxyribonucleotide triphosphates (dNTPs). As described above with respect to compositions, the mixture of primer nucleic acid molecules may comprise (i) one or more primer nucleic acid molecules having a degenerate nucleic acid sequence of 2 nucleotides, (ii) one or more primer nucleic acid molecules having a degenerate nucleic acid sequence of 3 nucleotides; (iii) one or more primer nucleic acid molecules having a degenerate nucleic acid sequence of 4 nucleotides, and/or (iv) one or more primer nucleic acid molecules having a degenerate nucleic acid sequence of 6 nucleotides in any suitable amounts. In some embodiments, the mixture may be comprised of equal amounts of UHP2, UHP4, and/or UHP6 (i.e., a 1:1:1 ratio). Other ratios of UHP2:UHP4:UHP6 that are encompassed by the present disclosure include, but are not limited to, 1:1:2, 1:2:1, 2:1:1, 1:1:3, 1:3:1, 3:1:1, 1:1:4, 1:4:1, 4:1:1, 1:1:5, 1:5:1, 5:1:1, 1:1:6, 1:6:1, 6:1:1, 1:1:7, 1:7:1, 7:1:1, 1:1:8, 1:8:1, 8:1:1, 1:1:9, 1:9:1, 9:1:1, 1:1:10, 1:10:1, 10:1:1, 1:1:15, 1:15:1, 15:1:1, 1:1:20, 1:20:1, and 20:1:1. An exemplary mixture comprises an 8:1:1 mole ratio of UHP2:UHP4:UHP6. In some embodiments, the mixture comprises DHP2 (i.e. UHP2), DHP3 (i.e. UHP3), DHP4 (i.e. UHP4), and DHP6 (i.e. UHP6). In some embodiments, the mixture comprises DHP2:DHP3:DHP4:UHP6 at a 1:1:7:1 molar ratio.

In addition to the universal hairpin primers described herein, the system desirably comprises other reagents necessary for carrying out reverse transcription and quantitative real-time PCR (RT-qPCR). Such reagents include, but are not limited to, a reverse transcriptase, deoxyribonucleotide triphosphates (dNTPs), a DNA polymerase, and one or more buffers. The terms “reverse transcriptase” and “RNA-dependent DNA polymerase,” may be used interchangeably to refer to a DNA polymerase enzyme that transcribes single-stranded RNA into DNA. In some embodiments, the reverse transcriptase may have intrinsic RNase H activity, which typically is favored in quantitative PCR applications because they enhance the melting of RNA-DNA duplex during the first cycles of PCR. A variety of reverse transcriptases suitable for RT-qPCR are known in the art and may be used in the disclosed systems and methods. For example, M-MLV reverse transcriptase from the Moloney murine leukemia virus or AMV reverse transcriptase from the avian myeloblastosis virus are typically used in quantitative RT-PCR applications. M-MLV reverse transcriptase is the preferred reverse transcriptase in cDNA synthesis for long messenger RNA (mRNA) templates (>5 kb) because the RNase H activity of M-MLV reverse transcriptase is weaker than the AMV reverse transcriptase (see, e.g., Mo et al., Methods Mol Biol., 926: 99-112 (2012)). Thermostable RNAse H-RTs also have been recently developed and may be used in connection with the systems and methods described herein.

The term “deoxyribonucleotide triphosphates (dNTPs)” generally refers to the four deoxyribonucleotides dATP, dCTP, dGTP and dTTP, which comprise the building blocks of DNA. dNTPs have a hydroxyl (—OH) group attached to the 3′ carbon of the deoxyribose sugar ring. Starting at the 3′ hydroxyl of the primer, DNA polymerase connects the incoming nucleotides to the growing DNA chain. When nucleotides are joined, the phosphate group attached to the 5′-carbon of the incoming nucleotide is linked to the 3′-hydroxyl group of the growing DNA chain. During the reaction the hydrogen ion (H⁺) on the 3′ hydroxyl group is released, as well as the two outer phosphate groups from the incoming dNTP.

The term “DNA polymerase,” as used herein, refers to the primary enzyme which catalyzes the formation of DNA from dTNPs, using single-stranded DNA as a template. DNA polymerases extend the DNA chain by adding nucleotides, one at a time, to the 3′ hydroxyl group at the end of the growing chain to the 5′ phosphate of nucleotide to be added. A variety of DNA polymerases suitable for RT-qPCR are known in the art and may be used in the disclosed systems and methods. In some embodiments, a thermostable DNA polymerase is used. Thermostable DNA polymerases can withstand high denaturation temperatures, and typically are divided into two groups: those with a 3′→5′ exonuclease (proofreading) activity, such as Pfu DNA polymerase, and those without the proofreading function, such as Taq DNA polymerase. Proofreading DNA polymerases are more accurate than nonproofreading polymerases due to the 3′→5′ exonuclease activity, which can remove a misincorporated nucleotide from a growing DNA chain. However, Taq DNA polymerase is the most commonly used enzyme because yields tend to be higher with a nonproofreading DNA polymerase.

Taq DNA polymerase is isolated from Thermus aquaticus and catalyzes the primer-dependent incorporation of nucleotides into duplex DNA in the 5′→3′ direction in the presence of Mg²⁺. The enzyme does not possess 3′→5′ exonuclease activity but has 5′→3′ exonuclease activity (see, e.g., Eckert, K. A. and Kunkel, T. A., Nucl. Acids Res., 18: 3739-44 (1990); Eckert, K. A. and Kunkel, T. A., PCR Methods Appl., 1: 17-24 (1991)). Tfl DNA polymerase catalyzes the primer-dependent polymerization of nucleotides into duplex DNA in the presence of Mg²⁺ (see, e.g., Gaensslen et al., J. Forensic Sci., 37: 6-20(1992)). Tth DNA polymerase catalyzes polymerization of nucleotides into duplex DNA in the 5′→3′ direction in the presence of MgCl2 (Myers, T. W. and Gelfand, D. H., Biochemistry, 30: 7661-6 (1991); Ruttimann et al., Eur. J. Biochem., 149: 41-46 (1985)). Tth DNA polymerase exhibits a 5′→3′ exonuclease activity but lacks detectable 3′→5′ exonuclease activity. Pfu DNA polymerase has one of the lowest error rates of all known thermophilic DNA polymerases used for amplification due to its high 3′→5′ exonuclease activity.

The disclosure also provides a method of quantifying RNA in a sample. In some embodiments, the method of quantifying RNA in the sample comprises (a) contacting the sample with the above-described system under conditions whereby the primer nucleic acid molecule, or mixture of primer nucleic acid molecules, hybridizes to RNA present in the sample and reverse transcription of the RNA occurs; and (b) amplifying and quantifying the reverse transcribed RNA using quantitative real-time PCR. In some embodiments, the RNA is miRNA. In some embodiments, the RNA is lncRNA. In some embodiments, the RNA is mRNA.

The terms “sample” or “biological sample” as used herein, refer to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the sample desirably comprises mammalian cells, such as human cells.

Amplification of reverse transcribed RNA (e.g. miRNA, mRNA, lncRNA) is mediated by DNA polymerase under routine cycling conditions and temperatures. The resulting amplified DNA may be quantified using any suitable method. Such methods may involve, for example, gene-specific fluorescent probes or specific double strand (ds) DNA binding agents based on fluorescence resonance energy transfer (FRET). An exemplary probe-based detection system is TAQMAN® (Applied Biosystems), which makes use of the 5′-3′ exonuclease activity of Taq polymerase to quantitate target sequences in the samples. Probe hydrolysis separates fluorophore and quencher and results in an increased fluorescence signal called “Forster type energy transfer.” Other detection methods that may be used in the disclosed method include non-sequence specific fluorescent intercalating dsDNA binding dyes, such as SYBR Green I (Molecular Probes) or ethidium bromide.

The levels of expressed RNA (e.g., expressed mature miRNA) may be measured by absolute or relative quantitative RT-PCR. Absolute quantification relates the PCR signal to input copy number using a calibration curve, while relative quantification measures the relative change in miRNA expression levels. Relative quantification is generally easier to perform than absolute quantification because a calibration curve is not necessary. Relative quantification is based on the expression levels of a target gene versus a housekeeping gene (reference or control gene) and typically is sufficient for most investigations of physiological changes in gene expression levels. Various mathematical models have been established to calculate the expression of a target gene in relation to an adequate reference gene various. Such calculations may be based on the comparison of the distinct cycle determined by various methods, e.g., crossing points (CP) and threshold values (Ct) at a constant level of fluorescence; or CP acquisition according to established mathematic algorithms (see, e.g., Tichopad et al., Molecular and Cellular Probes, 18: 45-50 (2004); and Tichopad et al., Biotechn Lett, 24: 2053-2056 (2003)). Methods for quantifying RNA in RT-qPCR are further described in, e.g., Wong, M. L. and J. F. Medrano, BioTechniques, 39: 75-85 (July 2005).

Systems and methods for conducting RT-qPCR are known in the art (see, e.g., Kroh et al., Methods, 50(4): 298-301 (2010); Mo et al., supra). Commercially available real-time PCR systems that may be utilized in connection with the present disclosure include, for example, STEPONE™ & STEPONEPLUS™ real-time PCR instruments and QUANTSTUDIO™ real-time PCR system (all from Applied Biosystems); LIGHTCYCLER® instruments (Roche); CFX Connect and iQ5 & MyiQ Cycler (all from BioRad); Mx3000 and Mx3005P (Agilent Technologies); Eco qPCR (Illumina); and PikoReal real-time PCR system (Thermo Fisher Scientific).

The following examples further illustrate the invention but, of course, should not be construed as in any way limiting its scope.

### EXAMPLES

Mature microRNAs (miRNAs or miRs) are a group of evolutionarily conserved endogenous, single-stranded, small noncoding RNAs with an average length of 22 nucleotides (nt) (1-4). The biogenesis of miRNAs starts with their transcription into primary miRNA (pri-miRNA) transcripts, which are subsequently processed into precursor miRNAs (pre-miRNAs), and finally into mature miRNAs through DROSHA/DICER cleavage machinery (3,4). Mechanistically, miRNAs are associated with Argonaute (AGO) proteins to form the so-called RNA-induced silencing complex (RISC) and post-transcriptionally modulate gene expression by guiding AGOs to complementary regions of target mRNAs to repress their translation or regulate degradation (3,4). It has been shown that miRNAs exhibit tissue-specific expression patterns (3). Pri-miRNAs can generate a single mature miRNA or clusters of related miRNAs (3). Furthermore, miRNAs can be grouped into families based on the similarity of their seed sequences, which comprise 2-8 nucleotides (counting from the 5′ end) and are primarily responsible for miRNA targeting of mRNAs (3). Emerging evidence has shown that miRNAs are essential regulators of numerous key cellular processes, including apoptosis, proliferation, and differentiation, and dysregulation of miRNAs may lead to the development of human diseases such as cancer and other chronic and metabolic disorders (3,4).

According to the world's largest collection of miRNA data, the miRNA Registry Databases miRBase (mirbase.org), the human genome encodes 2,654 mature microRNAs (1,908 in mice and 728 in rats) (miRBase v.22) (5), although GENCODE (v.29) documents more than 200,000 transcripts, including isoforms with slight variations (6). Another recently established miRNA candidates database, miRCarta, lists 12,857 human miRNA precursors (7). However, only approximately 2,300 true human mature miRNAs have been extrapolated, 1,115 of which are currently annotated in miRBase V22 (8). The main reason that many miRNAs are not classified as “high confidence” is the lack of expression data. Additionally, the abundance of different miRNAs in different cells and tissues varies drastically from 0 to about 1.4×10⁵ reads per million (RPM) (5). In fact, 1,225 human miRNAs (64%) do not have ≥20 reads associated with each arm in the datasets and thus cannot be confidently annotated (5).

Given the fact that miRNA expression levels vary significantly in different cells and tissues, accurate miRNA quantification is critical to assess the biological functions and possible pathogenic roles of miRNAs. Numerous techniques have been devised to detect miRNA expression under various physiological and pathological conditions (9-12). In general, miRNA detection methods include (1) conventional techniques such as Northern blotting, microarray, in situ hybridization, and quantitative reverse transcription (RT) PCR (RT-qPCR); (2) biosensor techniques such as electrochemical-based detection, optical-based detection, and nanotube-based techniques; and (3) other emerging techniques including next-generation sequencing (NGS), and nucleic acid amplification techniques such as rolling circle amplification (RCA), duplex-specific nuclease (DSN)-based amplification, loop-mediated isothermal amplification (LAMP), exponential amplification reaction (EXPAR), and strand-displacement amplification (SDA) (9-12).

These techniques, however, involve long processing times, laborious procedures, low throughput, large sample size requirements, false positives, lack of sensitivity, and/or costly instrument requirements. Thus, there remains a need for systems and methods to quantify mature miRNA expression more accurately. In some aspects, exemplified herein are systems and methods for quantification of mature miRNA.

The following materials and methods were used in the experiments described in Examples 1-4.

### Cell Culture and Chemicals

Human HEK-293, human osteosarcoma 143B, and human melanoma A375 cells were obtained from the American Type Culture Collection (ATCC, Manassas, VA), and the immortalized human umbilical cord-derived mesenchymal stem cells (UC-MSCs) were previously described. All cells were cultured in DMEM supplemented with 10% fetal bovine serum (FBS, Gemini Bio-Products), 100 U/ml penicillin, and 100 μg/ml streptomycin at 37° C. in 5% CO2 as described previously (16-18). Unless indicated otherwise, other chemicals were purchased from ThermoFisher Scientific (Waltham, MA) or Millipore Sigma (St. Louis, MO).

Design and Synthesis of miRNA-Specific Hairpin Primers (MsHP) and Universal Hairpin Primers (UHPs) for Reverse Transcription Reactions

The design of hairpin or stem-loop primers for reverse transcription of miRNA samples is illustrated in FIG. 1. All DNA oligonucleotides including qPCR primers were synthesized by Millipore Sigma. Synthetic mature miRNAs HSA-LET7d, HSA-LET7e, HSA-LET7i and HSA-LET7g were ordered from the Integrated DNA Technologies (IDT; Coralville, IA). The oligonucleotide sequences and their utilities are summarized in Table 1.

Total RNA Isolation and Small RNA (sRNA) (<200 nt) Purification

Total RNA was isolated from exponentially growing HEK-293 cells using the NucleoZOL RNA Isolation kit (Takara Bio USA, Mountain View, CA) according to the manufacturer's instructions as described (19-21). To purify small RNA (<200 nt), magnetic bead-based size selection was performed with the commercially available Mag-Bind® TotalPure NGS magnetic beads (Mag-Bind beads, Omega Bio-tek, Inc., Norcross, GA) as described previously (22). Briefly, 5 μg of total RNA were dissolved in 20 μl RNase-free molecular biology grade ddH2O and mixed with 20 μl Mag-Bind beads (i.e., RNA:Beads, volume/volume ratio of 1:1). The RNA/magnetic beads mixture was incubated at room temperature for 10 min. The mixture was subjected to a magnet, and the small RNA (<200 nt)-containing supernatant was collected while the large transcripts (>200 nt) bound to beads and were discarded. The collected small RNA was subjected to PC8 phenol/chloroform extraction, followed by ethanol precipitation. The recovered small RNA was dissolved in 20 μl RNase-free molecular biology grade ddH2O for reverse transcription reactions, or kept at −80° C.

Characterization and Quantification of the Purified Small RNA (sRNA)

After magnetic bead-based size selection, the recovered small RNA collection was assessed by using the Agilent 2100 Bioanalyzer (Santa Clara, CA) as described (23). Briefly, the recovered small RNA and total RNA samples (1.0 μl each) were loaded onto the Bioanalyzer RNA Nano Chips, along with size marker. The chip was subjected to electrophoresis according to the manufacturer's instructions. The integrity and quantity of the RNA samples were visualized in both gel images and electropherograms.

### Reverse Transcription Reactions (RT) Using Hairpin (Stem-Loop) Primers

The 14 miRNA-specific hairpin primers (MsHPs) and four universal hairpin primers (UHPs) were dissolved in RNase-free ddH2O at 1.0 μg/μl. The MsHP pool was created by mixing 10 μl of each MsHP. For RT reactions, one microgram of total RNA or 0.1 μg of purified sRNA (in 10 μl ddH2O) was mixed with 2.0 μl of MsHP pool, or UHPs (i.e., UHP2, UHP3, UHP4 and UHP6), and annealed at 70° C. for 5 min. After being cooled down on ice, each RNA/hairpin primer mixture was supplemented with 0.5 μl of RNase Inhibitor (New England Biolabs, or NEB, Ipswich, MA), 2 μl of 10× RT Buffer (NEB), 2 μl of 10 mM dNTPs, 0.5 μl of M-MuLV Reverse Transcriptase (NEB), and 3 μl RNase-free ddH2O. The RT reactions were kept at 25° C. for 10 min, and then 37° C. for 30 min. 80 μl of ddH2O were added to the RT products, which served as qPCR templates with further dilutions and were kept at −80° C.

### Touchdown Quantitative Real-Time PCR (TqPCR) and Data Analysis

To increase the annealing temperature, a sequence of AGCC was added to the first 17 nt of all mature miRs, and used as miRNA qPCR forward primers. The oligonucleotide 5′-GTG CAG GGT CCG AGG TCC GAG-3′, which is derived from the hairpin or stem-loop structure, was used as a common miRNA qPCR reverse primer. Primers for the reference transcript human 5s ribosomal RNA were designed using the Primer3Plus program. The TqPCR reactions were set up by using the 2× Forget-Me-Not™ EvaGreen qPCR Master Mix (Biotium, Fremont, CA), and carried out by using CFX-Connect (Bio-Rad) as previously described (24-27). The TqPCR cycling program was as follows: 95° C.×3′ for one cycle; 95° C.×20″, 66° C.×10″, for 4 cycles by decreasing 3° C. per cycle; 95° C.×20″, 55° C.×10″, 70° cx 1″, followed by plate read, for 40 cycles.

Five-fold serial dilutions were performed to determine the amplification efficiency for each qPCR primer pairs. No template control (NTC) was used as a negative control. All reactions were done in triplicate. To quantitatively assess the Quantification cycle (Cq) deviation from miRNA-specific hairpin primer (MsHP) group, ΔCq values were calculated for the UHP groups by subtracting individual average Cq value from respective Cq value for the MsHP group: ΔCq=average Cq (MsHP)−average Cq (UHP).

### Data Analysis and Statistical Evaluation

All qPCR reactions were done in triplicate and/or in three independent batches of experiments. The Linear Mixed-effects Models fitted by restricted maximum likelihood (REML) with the lme4 R package was employed to identify the fittest UHP, compared with the Cq values yielded by using MsHP. The nonparametric Kruskal-Wallis test with pairwise comparisons using Wilcoxon rank sum exact test was carried out to assess the statistical difference among the ΔCq values of the four UHPs, relative to that of the miRNA-specific hairpin primer group. Linear regression and correlation coefficient analysis was carried out to assess the effect of long transcripts on miRNA quantification. Whenever a comparison was made, a p-value<0.05 was considered statistically significant. All statistical analyses were performed using R Statistical Software (version 4.0.4, 2021; R Foundation for Statistical Computing, Vienna, Austria).

### Example 1

This example demonstrates that a universal hairpin primer (UHP) system provides a broad dynamic range of amplification in qPCR-based detection of has been a robust system in miRNA expression.

As discussed above, the MsHP system is not cost-effective for large scale and/or high throughput analysis of multiple miRNAs simultaneously. To overcome this limitation, a novel universal hairpin primer (UHP) system for RT-PCR-based miRNA quantification was designed (FIG. 1B). In this system, four universal hairpin primers (UHPs) were tested, designated as UHP2, UHP3, UHP4 and UHP6, which share the same hairpin sequence as that of MsHP's (SEQ ID NO: 1), except that they contain 2, 3, 4, and 6 randomized nucleotides at the 3′-end of the stem sequence. Their hairpin structures are illustrated in FIG. 1B.

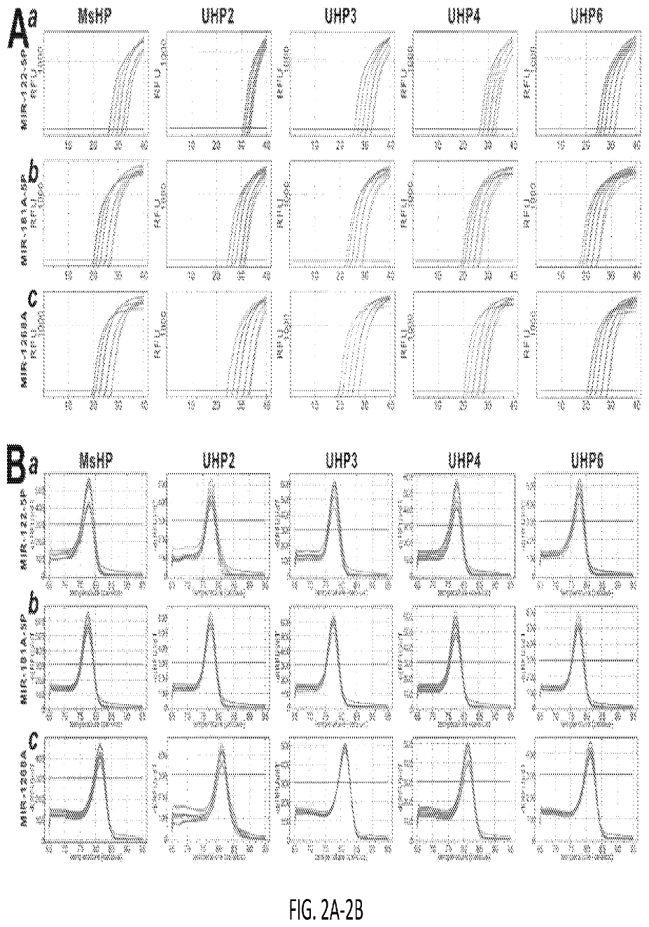

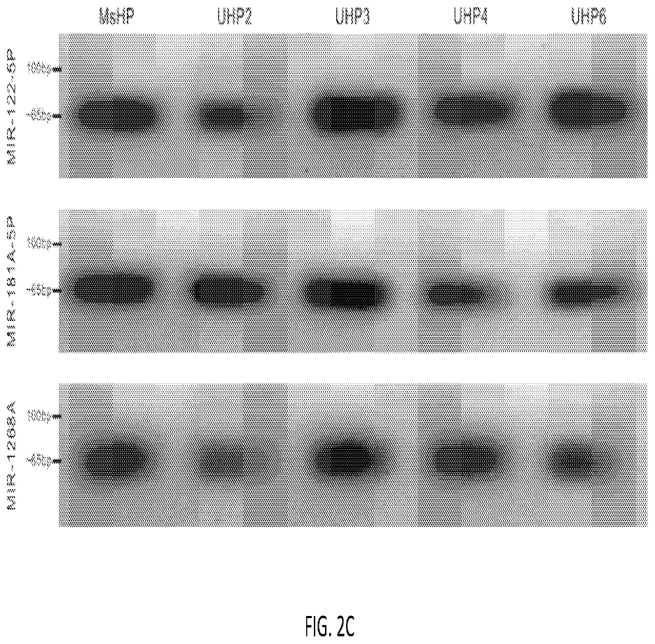

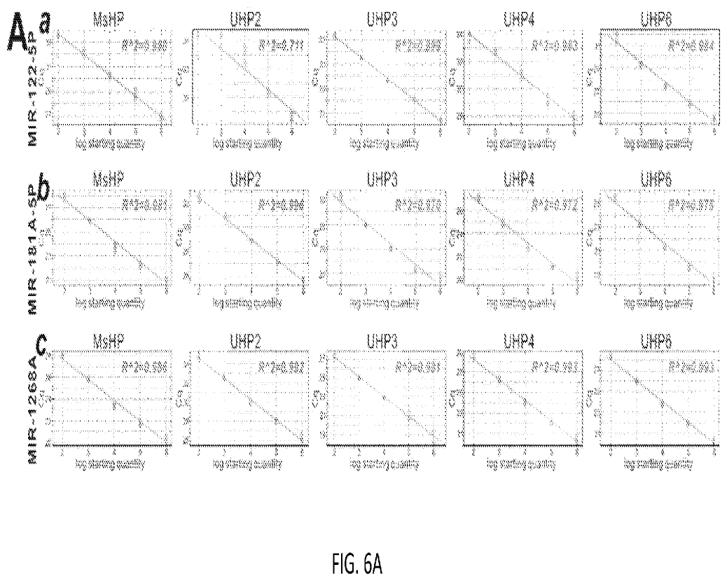

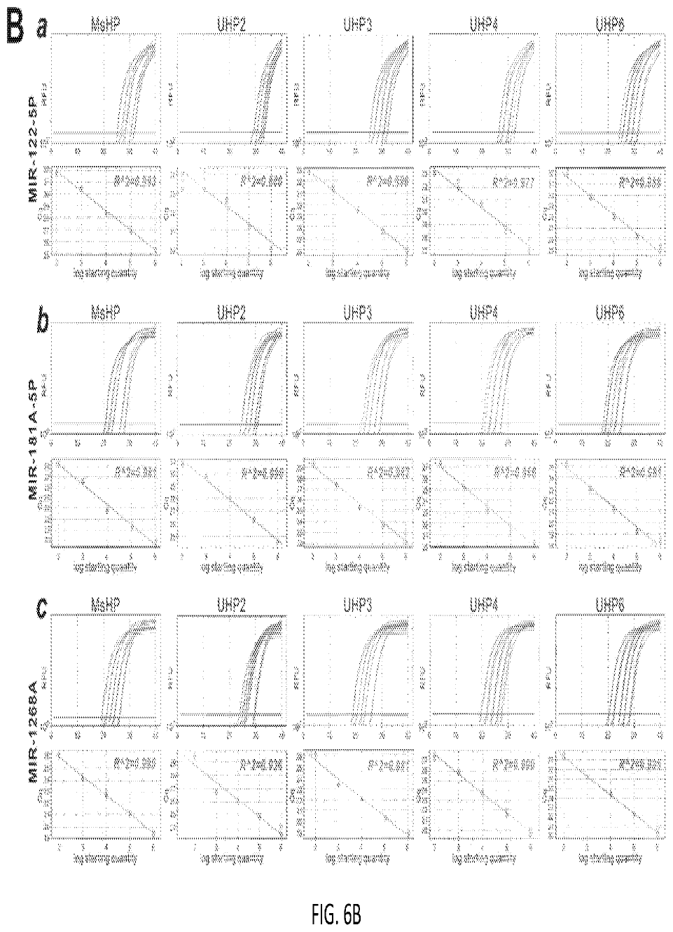

The sensitivity and specificity of the four UHPs as reverse transcription (RT) primers was tested, in comparison with that of the MsHP pool. The RT products were prepared with the four UHPs and MsHP, and then 4-fold serially diluted. For practical reasons, three representative miRNAs, i.e., HSA-MIR-122-5P (FIG. 1A-a), HSA-MIR-181A-5P (FIG. 1A-b), and HSA-MIR-11268A (FIG. 1A-c), were selected and their expression was quantified in the prepared RT products. The three selected miRNAs displayed proper amplification curves in a template concentration-dependent fashion (FIG. 2A, panels a, b, and c). However, when compared with the MsHP group, the amplification curves for the UHP2 group were right-shifted, while the amplification curves for the UHP6 group were left-shifted, at least for HSA-MIR-122-5P and HSA-MIR-181A-5P (FIG. 1A-ab). Nonetheless, the UHPs yielded excellent standard curves for the three miRNAs tested with R² value>0.97, except for MIR-122-5P primed with UHP2 (R² value=0.711) (FIG. 6A, panels a, b, and c). The melt curves indicated that all UHPs generated a single peak (FIG. 2B), and agarose gel analysis also confirmed that all UHP groups generated a single band with the same size as that of the MsHP groups' (FIG. 2C). Alternatively, a serial dilution of total RNA was performed, followed by reverse transcription using MsHP and four UHP primers. The RT products were subjected to TqPCR analysis using specific forward primers for HSA-MIR-122-5P, HSA-MIR-181A-5P, and HSA-MIR-1268A.

These results demonstrate that: 1) the four UHPs were effective and specific in initiating the RT reactions for miRNA quantification; and 2) the miRNA qPCR primer pairs consisting of miRNA-specific forward primers and the common reverse primer derived from the hairpin provided a reasonable dynamic range of detection with high amplification efficiency.

### Example 2

This example demonstrates the degenerate tetramer in UHP4 closely recapitulates MsHP pool in miRNA qualification.

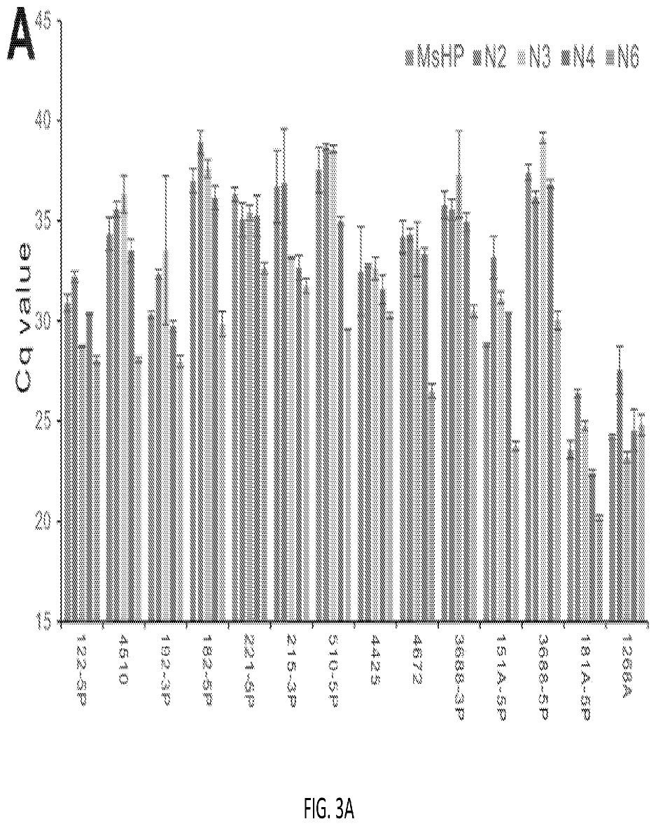

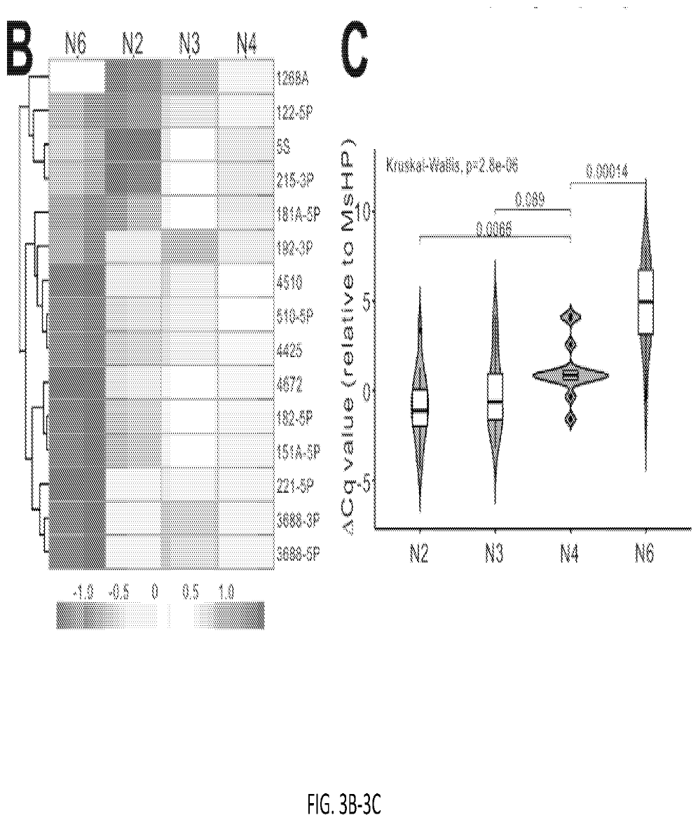

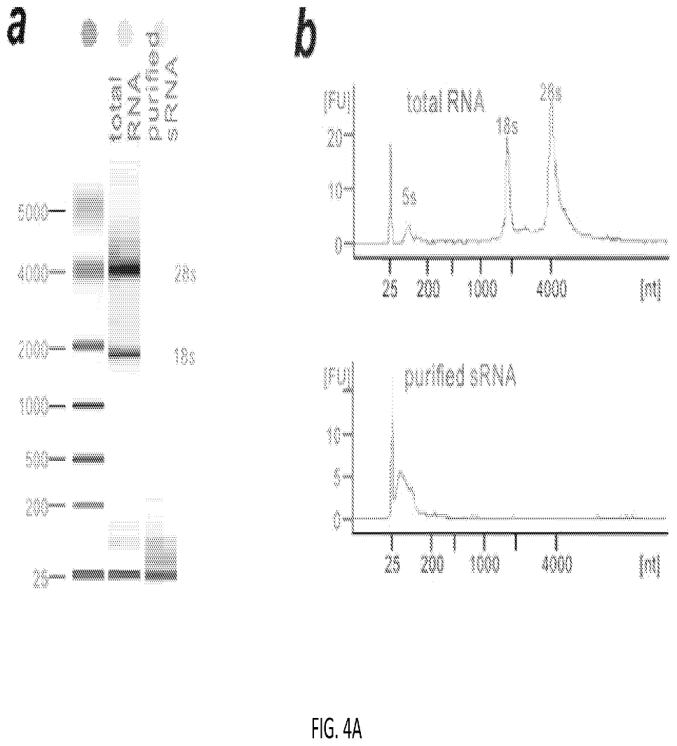

As shown above, while the four UHPs were able to detect miRNA expression with high sensitivity and specificity, it is important to determine whether their amplifications represent the actual expression levels of the tested miRNAs as defined by their miRNA-specific hairpin primers. To ensure the validity of such fit test assays, a panel of 14 miRNAs was chosen with a wide range of expression levels. The RT products were prepared from total RNA samples with the MsHP pool, UHP2, UHP3, UHP4 and UHP6 primers, and subjected to TqPCR as previously described (24), using the 14 miRNA-specific forward primers and a common reverse primer. For the RT products derived from MsHPs and four UHPs, five of the 14 analyzed miRNAs exhibited the Cq values relatively close to those of the respective MsHPs, including HSAMIR-122-5p, HSAMIR-192-3p, HSAMIR-221-5p, HSAMIR-4425, and HSAMIR-1268A (FIG. 3A). However, the Cq values of the remaining nine miRNAs had significant deviations from that of the respective MsHPs, and in particular, the UHP6 group seemingly yielded significantly lower Cq values, compared with respective MsHPs (FIG. 3A). Furthermore, the Linear Mixed-effects Models fitted by restricted maximum likelihood (REML) were conducted which identified that UHP4 yielded Cq values that were the closest to that of respective MsHPs.

The ΔCq values were further calculated relative to respective MsHPs for the UHPs. Heatmap clustering analysis indicated that 13 of the 14 tested miRNAs have positive ΔCq values in the UHP6 group, indicating an overestimation of miRNA expression compared to that of respective MsHPs (FIG. 3B). Conversely, 11 of the 14 tested miRNAs have negative ΔCq values in the UHP2 group, suggesting that the miRNA expression may be underestimated in this group, compared with that of the MsHPs (FIG. 3B). For the UHP3 group, while 9 of the 14 miRNAs have negative ΔCq values and 5 have positive ΔCq values, the range of the ΔCq values is significantly narrower, and 10 of the 14 miRNA have the ΔCq values within ±2 range (FIG. 3B). Consistent with the conclusion of the Linear Mixed-effects Models fit test, the UHP4 group yields the smallest overall ΔCq values, and 11 of the 14 miRNAs have the ΔCq values of less than 1.0, compared with that of respective MsHPs (FIG. 3B), suggesting that UHP4 may be the best surrogate for MsHPs in RT-PCR-based miRNA quantification.

The ΔCq data also was analyzed using the box and whisker plot. The nonparametric Kruskal-Wallis analysis indicates there was a statistical difference among the four UHPs (p value=2.8e-6). As shown in FIG. 3C, the medians (shown in the middle quartile) for UHP2, UHP3 and UHP4 were close to “0”, while the median for UHP6 deviated significantly from “0” (FIG. 3C). As expected, UHP4 group yielded the tightest box of the middle 50%, and the median was the closest to “0” among the four UHP groups, whereas the whiskers were the shortest among the four UHP groups (FIG. 3C), indicating lower variabilities outside the upper and lower quartiles than other UHP groups. Interestingly, the difference in data distributions between UHP3 and UHP4 was not statistically significant (FIG. 3C). Collectively, these results demonstrate that the UHP4 is the most approximate of the tested MsHPs in RT-PCR based miRNA quantification.

### Example 3

This example demonstrates that the presence of ribosomal RNAs and long transcripts does not affect the UHP-based qPCR quantification of miRNA expression.

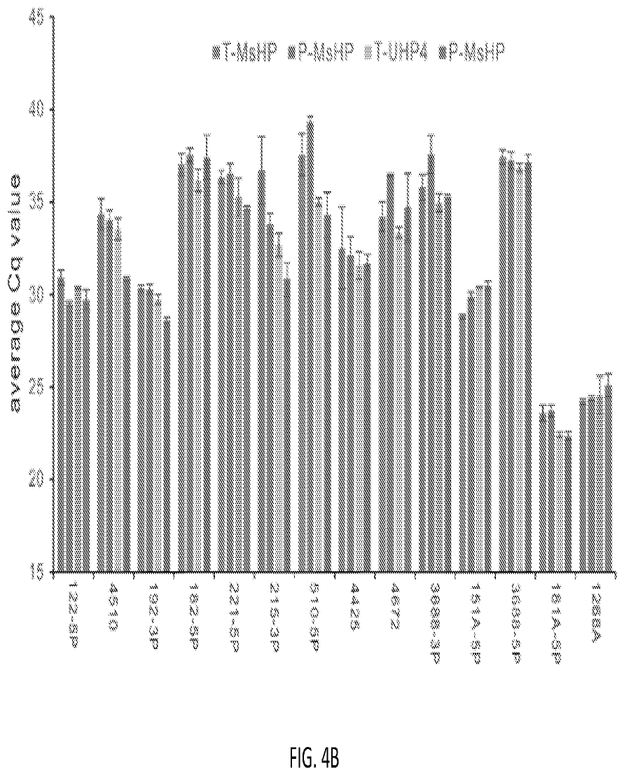

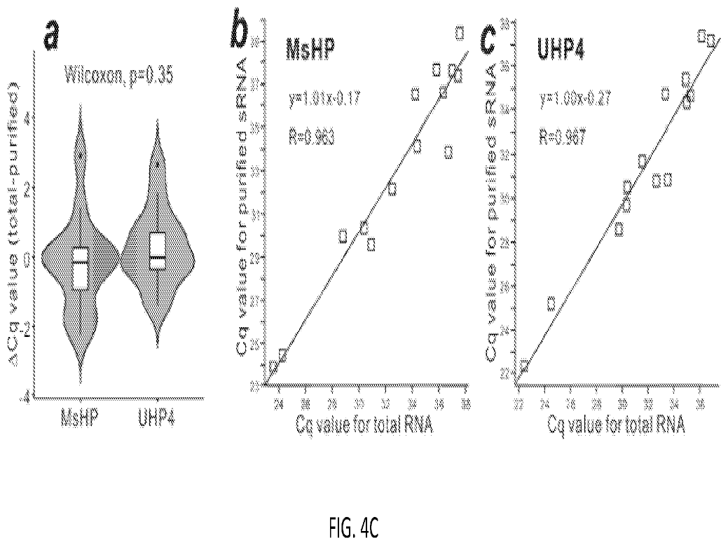

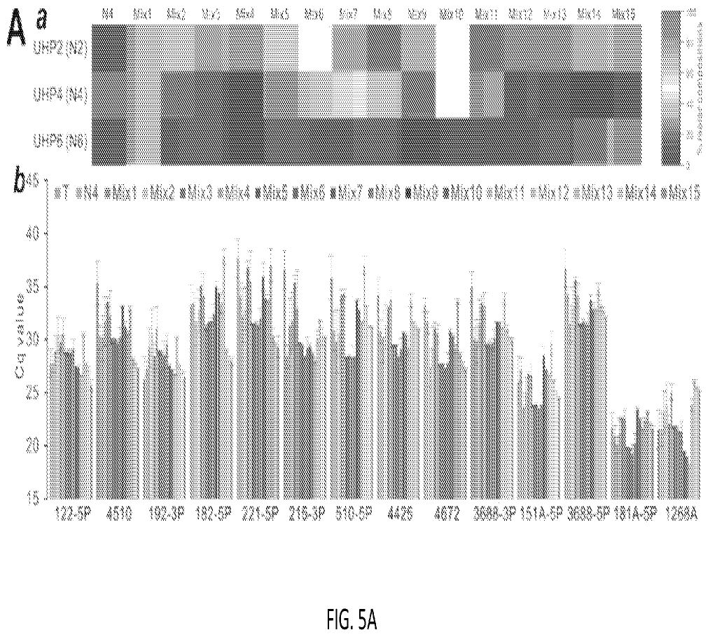

Many miRNA quantification protocols require the purification of small RNAs using commercially available kits. In this study, the 3′-end randomized hairpin primers or UHPs were used for RT reactions. It was conceivable that the UHPs could produce large amounts of non-miRNA-related RT products from rRNAs and long transcripts and lead to decreased sensitivity and specificity in miRNA quantification. To test whether such adverse effect may exist, a side-by-side comparison study of miRNA quantification was conducted by using the RT products prepared from total RNA and purified small RNA (sRNA) samples. A validated protocol was employed to separate different sizes of nucleic acids through a commercially available size selection magnetic beads system (22,23), and remove RNA species larger than 200 nt (FIG. 4A, panel a). The recovered sRNAs were smaller than 200 nt based on the results from Agilent 2100 Bioanalyzer assays (FIG. 4A, panel b).

Using the purified sRNA sample along with its corresponding total RNA sample, RT reactions were performed using MsHIP and UHP4 primers. The average Cq values of the 14 miRNAs were at similar levels, while certain variations were observed in a few miRNAs, albeit without statistical significance (p>0.20) (FIG. 4B). The box and whisker plot analysis indicated that the ΔCq values between total RNA samples and purified sRNAs for the 14 tested miRNAs were tightly centered at the “0” position, and the nonparametric Wilcoxon signed-rank test found no statistical difference (p=0.35) (FIG. 4C, panel a). Furthermore, linear regression and correlation coefficient analysis indicated that average Cq values of the 14 tested miRNAs were highly correlated between total RNA and purified sRNA samples for both MsHP and UHP4 primer groups (FIG. 4C, panels bc).

These results demonstrate that the presence of ribosomal RNAs and long transcripts do not significantly affect the UHP-based RT-qPCR quantification of miRNA expression in biological samples.

### Example 4

This example describes the identification and characterization of an optimized UHP (OUHP) cocktail that serves as a surrogate for miRNA-specific hairpin primers in high throughput miRNA quantification.

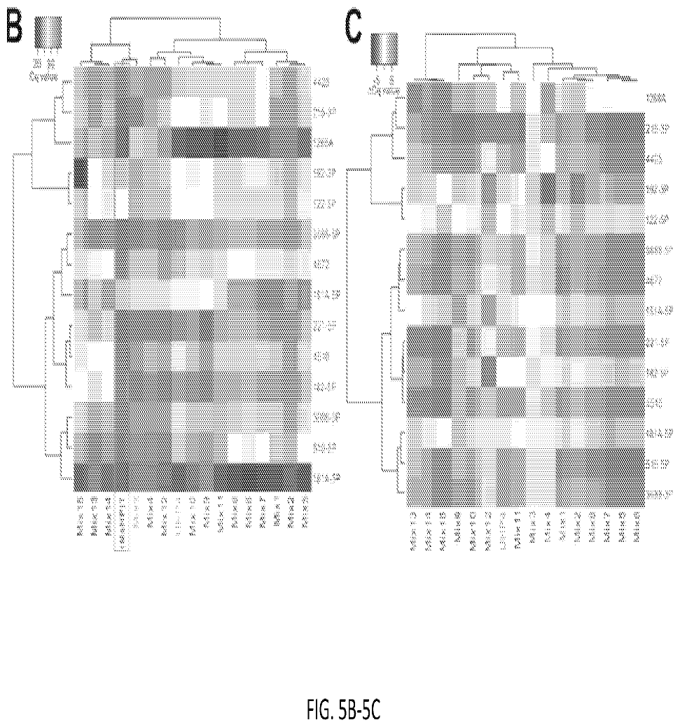

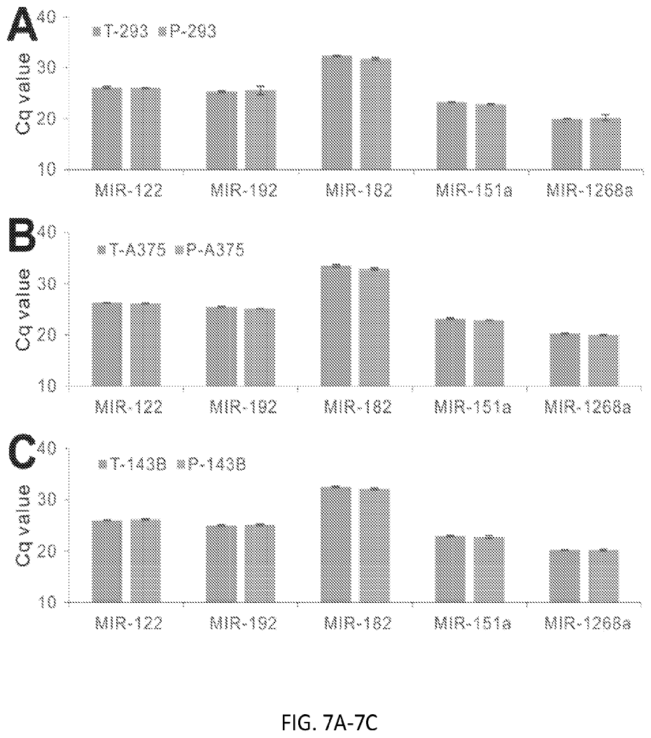

While the results presented in FIG. 3 indicate that the tetramer UHP4 closely recapitulated the Cq values obtained from the miRNA-specific hairpin primer-initiated RT products, UHP4 tended to overestimate miRNA expression in general. In order to develop an optimized UHP (OUHP) to serve as a faithful MsHP surrogate, a panel of 15 UHP formulations was generated, namely Mix1 through Mix15, by mixing UHP2, UHP4 and/or UHP6 at various molar compositions in percentages (FIG. 5A, panel a; and FIG. 7A). The resultant Cq values for 14 miRNAs were subsequently assessed in comparison with that of respective MsHP's (FIG. 5A, panel b). Heatmap clustering analysis of the Cq values of the 4 tested miRNAs revealed that Mix3 was clustered together with MsHP, while Mix4 and Mix12 were clustered closely with UHP4 (FIG. 5B).

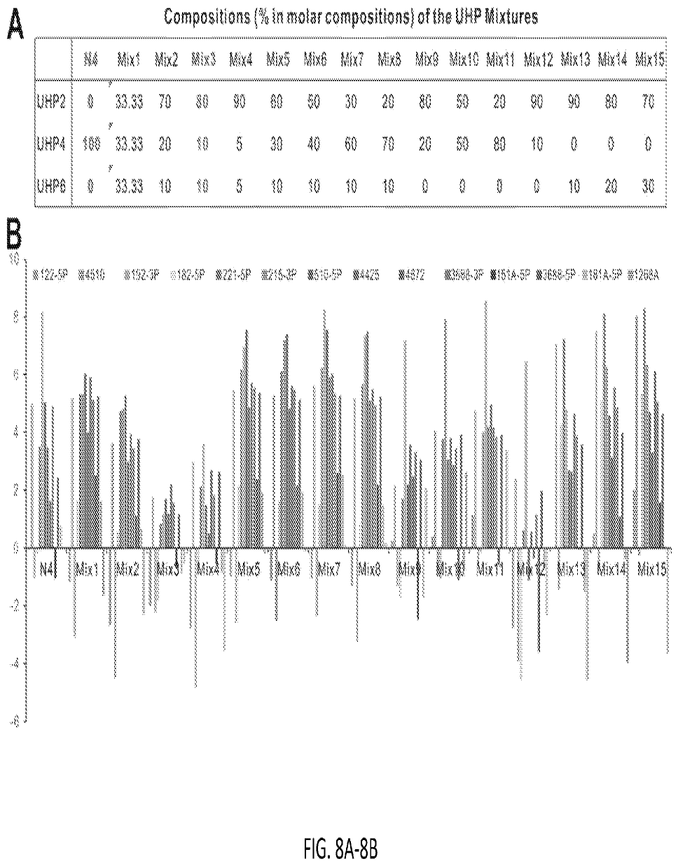

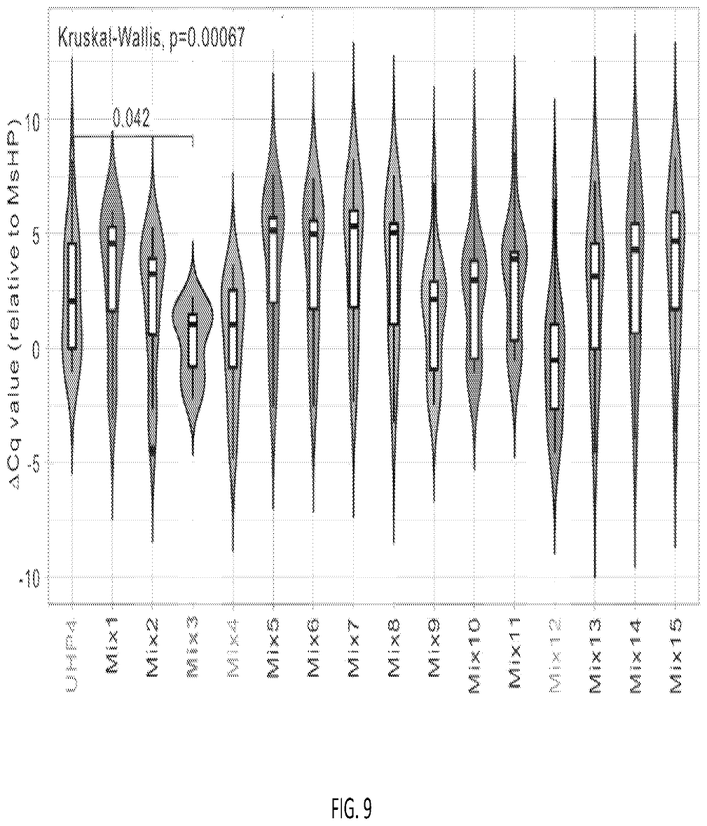

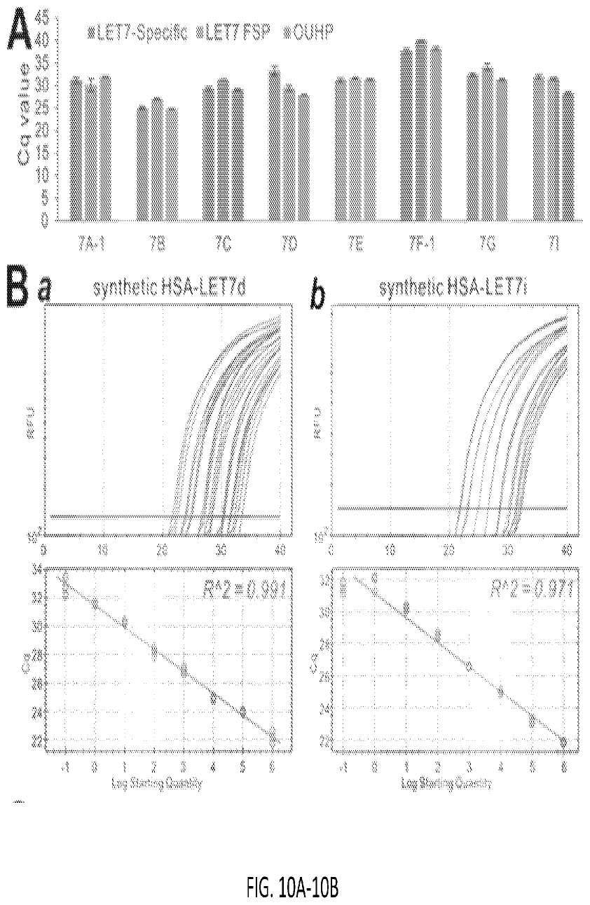

The ΔCq values relative to MsHPs were further analyzed for the 14 tested miRNAs by the 14 cocktail mixtures, as well as by UHP4. Heatmap clustering analysis of the ΔCq values indicated that the Mix3 group yielded the smallest deviations from zero among all 15 cocktail groups and the UHP4 group, while most of the other groups tended to significantly overestimate the levels of miRNA expression (FIG. 5C). A direct plot of the ΔCq values also revealed that Mix3 group displayed the smallest fluctuations around the “zero” axis (FIG. 7B), which was further confirmed by Boxplot analysis (FIG. 8). The distributions and variations of the ΔCq values between Mix3 and UHP4 were statistically significant, suggesting that UHP4 may be less optimal than Mix3 in representing MsHPs in miRNA quantification. Collectively, these results strongly suggest that Mix3 (i.e., UHP2:UHP4:UHP6=8:1:1, also designated as the optimized universal hairpin primer, or OUHP) serves as the best surrogate of MsHP for quantifying miRNA expression in a high throughput fashion.

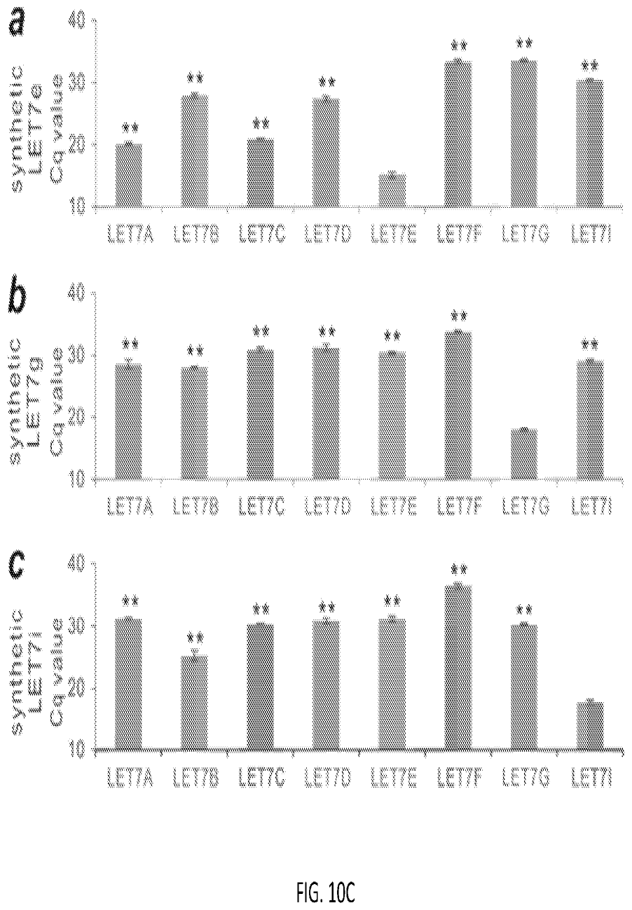

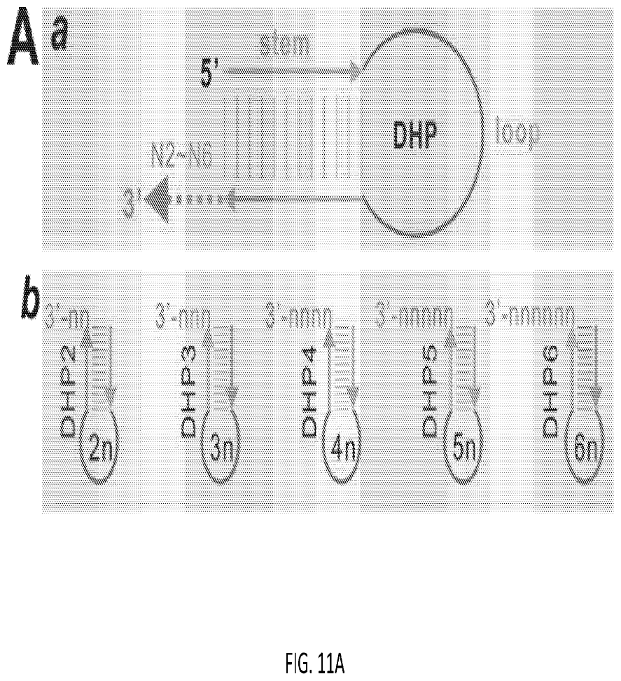

Lastly, the detection specificity was analyzed using the LET7 miRNA family. The OUHP primers could effectively detect the expression of all 8 members of the LET family with similar efficiency to that of LET7-specific hairpin primers (FIG. 10A). Using synthetic mature LET7d and LET7i, the OUHP primers detected a broad dynamic range of the mature LET7d and LET7i (FIG. 10B, panels ab). Furthermore, the synthetic mature LET7e, LET7g and LET7i were subjected to OUHP-mediated RT reactions, followed by qPCR analysis with LET7-specific forward primers. The Cq values were significantly lower in the synthetic LET7-specific forward primer group than that of other LET7 forward primer groups (FIG. 10C, panels abc). These results demonstrate that the OUHP primer system can provide significant detection specificity for even closely related miRNA family members.

### REFERENCES

All references, including publications, patent applications, and patents, cited herein are hereby incorporated by reference to the same extent as if each reference were individually and specifically indicated to be incorporated by reference and were set forth in its entirety herein.
1. Lee, R. C., Feinbaum, R. L. and Ambros, V. (1993) The C. elegans heterochronic gene lin-4 encodes small RNAs with antisense complementarity to lin-14. Cell, 75, 843-854.
2. Wightman, B., Ha, I. and Ruvkun, G. (1993) Posttranscriptional regulation of the heterochronic gene lin-14 by lin-4 mediates temporal pattern formation in C. elegans. Cell, 75, 855-862.
3. Gebert, L. F. R. and MacRae, I. J. (2019) Regulation of microRNA function in animals. Nat Rev Mol Cell Biol, 20, 21-37.
4. Treiber, T., Treiber, N. and Meister, G. (2019) Regulation of microRNA biogenesis and its crosstalk with other cellular pathways. Nat Rev Mol Cell Biol, 20, 5-20.
5. Kozomara, A., Birgaoanu, M. and Griffiths-Jones, S. (2019) miRBase: from microRNA sequences to function. Nucleic Acids Res, 47, D155-D162.
6. Plotnikova, O., Baranova, A. and Skoblov, M. (2019) Comprehensive Analysis of Human microRNA-mRNA Interactome. Front Genet, 10, 933.
7. Backes, C., Fehlmann, T., Kern, F., Kehl, T., Lenhof, H. P., Meese, E. and Keller, A. (2018) miRCarta: a central repository for collecting miRNA candidates. Nucleic Acids Res, 46, D160-D167.
8. Alles, J., Fehlmann, T., Fischer, U., Backes, C., Galata, V., Minet, M., Hart, M., Abu-Halima, M., Grasser, F. A., Lenhof, H. P. et al. (2019) An estimate of the total number of true human miRNAs. Nucleic Acids Res, 47, 3353-3364.
9. Hunt, E. A., Broyles, D., Head, T. and Deo, S. K. (2015) MicroRNA Detection: Current Technology and Research Strategies. Annu Rev Anal Chem (Palo Alto Calif), 8, 217-237.
10. Ye, J., Xu, M., Tian, X., Cai, S. and Zeng, S. (2019) Research advances in the detection of miRNA. J Pharm Anal, 9, 217-226.
11. Cheng, Y., Dong, L., Zhang, J., Zhao, Y. and Li, Z. (2018) Recent advances in microRNA detection. Analyst, 143, 1758-1774.
12. Ouyang, T., Liu, Z., Han, Z. and Ge, Q. (2019) MicroRNA Detection Specificity: Recent Advances and Future Perspective. Anal Chem, 91, 3179-3186.
13. Mestdagh, P., Hartmann, N., Baeriswyl, L., Andreasen, D., Bernard, N., Chen, C., Cheo, D., D'Andrade, P., DeMayo, M., Dennis, L. et al. (2014) Evaluation of quantitative miRNA expression platforms in the microRNA quality control (miRQC) study. Nat Methods, 11, 809-815.
14. Chen, C., Ridzon, D. A., Broomer, A. J., Zhou, Z., Lee, D. H., Nguyen, J. T., Barbisin, M., Xu, N. L., Mahuvakar, V. R., Andersen, M. R. et al. (2005) Real-time quantification of microRNAs by stem-loop RT-PCR. Nucleic Acids Res, 33, e179.
15. Shi, R. and Chiang, V. L. (2005) Facile means for quantifying microRNA expression by real-time PCR. BioTechniques, 39, 519-525.
16. Wu, X., Li, Z., Zhang, H., He, F., Qiao, M., Luo, H., Zhang, J., Zhang, M., Mao, Y., Wagstaff, W. et al. (2021) Modeling colorectal tumorigenesis using the organoids derived from conditionally immortalized mouse intestinal crypt cells (ciMICs). Genes & Diseases.
17. Fan, J., Feng, Y., Zhang, R., Zhang, W., Shu, Y., Zeng, Z., Huang, S., Zhang, L., Huang, B., Wu, D. et al. (2020) A simplified system for the effective expression and delivery of functional mature microRNAs in mammalian cells. Cancer Gene Ther, 27, 424-437.
18. Huang, X., Chen, Q., Luo, W., Pakvasa, M., Zhang, Y., Zheng, L., Li, S., Yang, Z., Zeng, H., Liang, F. et al. (2020) SATB2: A versatile transcriptional regulator of craniofacial and skeleton development, neurogenesis and tumorigenesis, and its applications in regenerative medicine. Genes & Diseases.
19. Liu, W., Deng, Z., Zeng, Z., Fan, J., Feng, Y., Wang, X., Cao, D., Zhang, B., Yang, L., Liu, B. et al. (2020) Highly expressed BMP9/GDF2 in postnatal mouse liver and lungs may account for its pleiotropic effects on stem cell differentiation, angiogenesis, tumor growth and metabolism. Genes Dis, 7, 235-244.
20. Zhang, L., Luo, Q., Shu, Y., Zeng, Z., Huang, B., Feng, Y., Zhang, B., Wang, X., Lei, Y., Ye, Z. et al. (2019) Transcriptomic landscape regulated by the 14 types of bone morphogenetic proteins (BMPs) in lineage commitment and differentiation of mesenchymal stem cells (MSCs). Genes Dis, 6, 258-275.
21. Fan, J., Wei, Q., Liao, J., Zou, Y., Song, D., Xiong, D., Ma, C., Hu, X., Qu, X., Chen, L. et al. (2017) Noncanonical Wnt signaling plays an important role in modulating canonical Wnt-regulated stemness, proliferation and terminal differentiation of hepatic progenitors. Oncotarget, 8, 27105-27119.
22. Wang, X., Zhao, L., Wu, X., Luo, H., Wu, D., Zhang, M., Zhang, J., Pakvasa, M., Wagstaff, W., He, F. et al. (2021) Development of a simplified and inexpensive RNA depletion method for plasmid DNA purification using size selection magnetic beads (SSMBs). Genes & Diseases, 8, 298-306.
23. Zeng, Z., Huang, B., Wang, X., Fan, J., Zhang, B., Yang, L., Feng, Y., Wu, X., Luo, H., Zhang, J. et al. (2020) A reverse transcriptase-mediated ribosomal RNA depletion (RTR2D) strategy for the cost-effective construction of RNA sequencing libraries. JAdv Res, 24, 239-250.
24. Zhang, Q., Wang, J., Deng, F., Yan, Z., Xia, Y., Wang, Z., Ye, J., Deng, Y., Zhang, Z., Qiao, M. et al. (2015) TqPCR: A Touchdown qPCR Assay with Significantly Improved Detection Sensitivity and Amplification Efficiency of SYBR Green qPCR. PloS one, 10, e0132666.
25. Huang, B., Huang, L. F., Zhao, L., Zeng, Z., Wang, X., Cao, D., Yang, L., Ye, Z., Chen, X., Liu, B. et al. (2020) Microvesicles (MIVs) secreted from adipose-derived stem cells (ADSCs) contain multiple microRNAs and promote the migration and invasion of endothelial cells. Genes Dis, 7, 225-234.
26. Zhong, J., Kang, Q., Cao, Y., He, B., Zhao, P., Gou, Y., Luo, Y., He, T. C. and Fan, J. (2021) BMP4 augments the survival of hepatocellular carcinoma (HCC) cells under hypoxia and hypoglycemia conditions by promoting the glycolysis pathway. American journal of cancer research, 11, 793-811.
27. An, L., Shi, Q., Zhu, Y., Wang, H., Peng, Q., Wu, J., Cheng, Y., Zhang, W., Yi, Y., Bao, Z. et al. (2020) Bone morphogenetic protein 4 (BMP4) promotes hepatic glycogen accumulation and reduces glucose level in hepatocytes through mTORC2 signaling pathway. Genes & Diseases.
28. Pritchard, C. C., Cheng, H. H. and Tewari, M. (2012) MicroRNA profiling: approaches and considerations. Nat Rev Genet, 13, 358-369.
29. Forero, D. A., Gonzalez-Giraldo, Y., Castro-Vega, L. J. and Barreto, G. E. (2019) qPCR-based methods for expression analysis of miRNAs. BioTechniques, 67, 192-199.
30. Schmittgen, T. D., Jiang, J., Liu, Q. and Yang, L. (2004) A high-throughput method to monitor the expression of microRNA precursors. Nucleic Acids Res, 32, e43.
31. Benes, V., Collier, P., Kordes, C., Stolte, J., Rausch, T., Muckentaler, M. U., Haussinger, D. and Castoldi, M. (2015) Identification of cytokine-induced modulation of microRNA expression and secretion as measured by a novel microRNA specific qPCR assay. Sci Rep, 5, 11590.
32. Jung, U., Jiang, X., Kaufmann, S. H. and Patzel, V. (2013) A universal TaqMan-based RT-PCR protocol for cost-efficient detection of small noncoding RNA. RNA (New York, N.Y.), 19, 1864-1873.
33. Tong, L., Xue, H., Xiong, L., Xiao, J. and Zhou, Y. (2015) Improved RT-PCR Assay to Quantitate the Pri-, Pre-, and Mature microRNAs with Higher Efficiency and Accuracy. Molecular biotechnology, 57, 939-946.
34. Honda, S. and Kirino, Y. (2015) Dumbbell-PCR: a method to quantify specific small RNA variants with a single nucleotide resolution at terminal sequences. Nucleic Acids Res, 43, e77.
35. Yang, L. H., Wang, S. L., Tang, L. L., Liu, B., Ye, W. L., Wang, L. L., Wang, Z. Y., Zhou, M. T. and Chen, B. C. (2014) Universal stem-loop primer method for screening and quantification of microRNA. PloS one, 9, e115293.

### Example 5

Reverse transcriptase-quantitative PCR (RT-qPCR or qPCR) is a commonly-used tool to quantify gene expression in life science. Among various RT-qPCR systems, SYBR Green-based qPCR is the most commonly used method to quantify coding and noncoding transcript expression thanks to its sensitivity and low cost, although it may lack specificity with limited detection ranges. On the contrary, fluorescent probe-based (e.g., TaqMan) qPCR offers high sensitivity and specificity with broad detection ranges, but there is high cost associated with fluorescent probe synthesis. Described herein is a universal quantification of expression (UniQE) system for cost-effective TaqMan qPCR analysis of coding and noncoding RNAs. Five degenerate hairpin primers (DHPs) were designed for RT reactions (i.e., DHP2 to DHP6), which have the same hairpin and universal TaqMan (U-TaqMan)-recognizing sequences, but contain 2 to 6 randomized nucleotides at the 3′-end. Through comparison with transcript-specific hairpin primers (TSPs) on 37 tester genes, the U-TaqMan qPCR analysis showed that DHP4 yielded quantification results closest to that of TSPs, whereas DHP6 overestimated and DHP2 underestimated the expression levels of the tester genes. Through linear regression-based machine learning analysis of 24 cocktails of DHPs (CODs) on the tester genes, an optimal DHP mix (i.e., COD24) was identified, which best recapitulated the TSPs in mRNA quantification. The COD24-mediated U-TaqMan qPCR system effectively quantified the expression levels of lncRNAs and miRNAs with high sensitivity and specificity. Collectively, these results demonstrate that the reported UniQE system provides a cost-effective tool for coding and noncoding transcriptomic quantification, which has broad applications in basic and translational research, as well as in clinical diagnostics.

### Materials and Methods

### Cell Culture and Chemicals:

Human HEK-293, human osteosarcoma lines 143B and SJSA1, and human melanoma lines A375, Mel-624 and Mel-888 cells were obtained from the American Type Culture Collection (ATCC, Manassas, VA). 293pTP and RAPA cells were derived from HEK-293 cells as previously described (17,18). The UC-MSC cells are reversely immortalized human umbilical cord mesenchymal stem cells as described (19). All cells were cultured in DMEM supplemented with 10% fetal bovine serum (FBS, Gemini Bio-Products), 100 U/ml penicillin, and 100 μg/ml streptomycin at 37° C. in 5% CO2 as described (20-23). M-MuLV Reverse Transcriptase and dNTPs were purchased from New England Biolabs (NEB, Ipswich, MA) and GenScript USA Inc (Catalog #C01581-10; Piscataway, NJ), respectively. Unless indicated otherwise, other chemicals were purchased from Thermo Fisher Scientific (Waltham, MA) or Millipore Sigma (St. Louis, MO).

### Construction, Amplification and Infection of Recombinant Adenoviruses Ad-Wnt1, Ad-Wnt3 and Ad-GFP:

Recombinant adenoviruses Ad-Wnt1 and Ad-Wnt3 were constructed by using the AdEasy technology as described (24,25). Briefly, the coding regions of mouse Wnt1 and Wnt3 were PCR amplified and subcloned into an adenoviral shuttle vector, followed by homologous recombination reactions with the adenoviral backbone vector pAdEasyl in BJ5183 cells. The resultant recombinant adenoviral plasmids were used to generate adenoviruses Ad-Wnt1 and Ad-Wnt3 in 293pTP cells, respectively. The adenovirus Ad-GFP was constructed by using the Gibson DNA Assembly-based OSCA system as described (26). All recombinant adenoviruses were packaged in 293pTP cells, and amplified to high titers in HEK-293, 293pTP or RAPA cells (17,18). The Ad-Wnt1 and Ad-Wnt3 also co-express GFP marker gene.

For the comparison analysis of Wnt-induced gene expression, subconfluent UC-MSC cells were infected with the same optimal titer of Ad-Wnt1, Ad-Wnt3, or Ad-GFP in the presence of polybrene (final concentration at 6 μg/mL) to enhance adenoviral transduction efficiency as described (27). At 48 h after infection, total RNA was isolated from the infected cells and subjected to RT reactions with the COD24 primers or the conventional hexamer. The RT products were used for qPCR analysis with U-TaqMan or conventional SYBR Green system.

### Transcriptomic Databases for Selecting Average Transcript Abundance Analysis of Human Transcriptome:

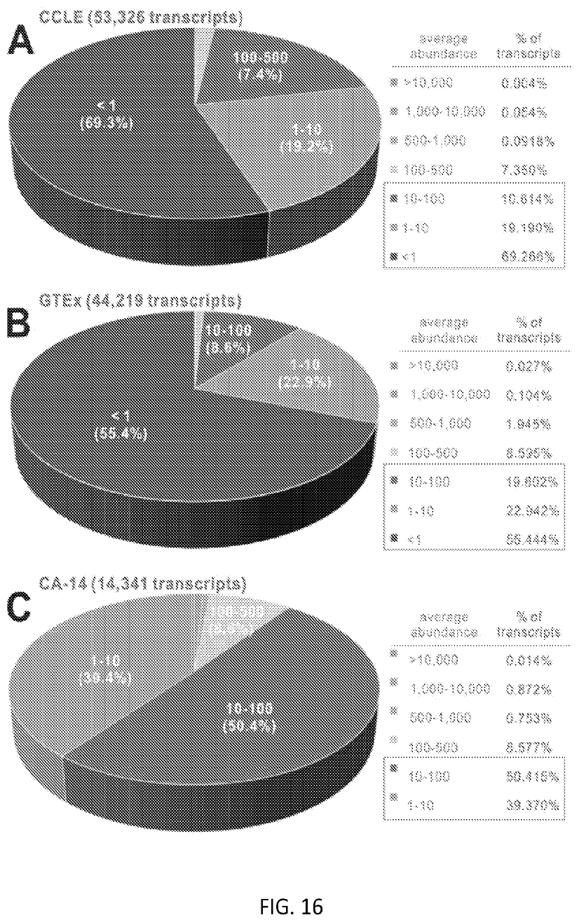

Three sources of human transcriptome databases were used to assess the average transcript abundance: (1) CCLE dataset: RNA-seq data (in reads per kilobase of transcript per million mapped reads, or RPKM) of 1,076 human cancer cells reported in the Broad Institute Cancer Cell Line Encyclopedia (CCLE; https://portals.broadinstitute.org/ccle) were retrieved from the UCSC XENA datasets (https://xenabrowser.net/datapages/); (2) GTEx dataset: RNA-seq data (in transcripts per million, or TPM) of 54 types of normal human tissues were retrieved from the Genotype-Tissue Expression (GTEx) project (https://gtexportal.org/home/); and (3) CA-14 dataset: RNA-seq data (in transcripts per million, or TPM) of 14 human cancer lines (including osteosarcoma, melanoma, and colorectal cancer lines) were retrieved from our homemade RNA-seq dataset in unrelated studies. We analyzed average transcript (mostly mRNA) abundance from the above three sources: CCLE dataset, GTEx dataset, and CA-14 dataset. A panel of 37 tester genes (Table 2) was selected based on the distributions of transcript abundance as shown in FIG. 16.

TABLE 2
List of selected Tester Genes

| | | | | GC | Gene | CCLE | GTEx | CA-14 |
|---|---|---|---|---|---|---|---|---|
| Symbol | Accession | ID | Length | content | ID | (RPKM) | (TPM) | (TPM) |
| GAPDH | NM_002046.7 | ENSG00000111640 | 2396 | 0.598 | 2597 | 1872.73 | 1309.36 | 6450 |
| RPS13 | NM_001017.3 | ENSG00000110700 | 2115 | 0.454 | 6207 | 558.06 | 994.86 | 5061 |
| PPIA | NM_021130.5 | ENSG00000196262 | 6780 | 0.467 | 5478 | 426.15 | 227.85 | 2745 |
| EIF3E | NM_001568.3 | ENSG00000104408 | 37761 | 0.372 | 3646 | 102.25 | 132.67 | 463 |
| VDAC1 | NM_003374.3 | ENSG00000213585 | 3049 | 0.558 | 7416 | 121.88 | 113.54 | 279 |
| HNRNPL | NM_001533.3 | ENSG00000104824 | 6492 | 0.571 | 3191 | 101.60 | 153.01 | 204 |
| RER1 | NM_007033.5 | ENSG00000157916 | 5689 | 0.536 | 11079 | 23.32 | 51.90 | 101 |
| NDUFC1 | NM_002494.3 | ENSG00000109390 | 2994 | 0.407 | 4717 | 20.81 | 49.60 | 99 |
| SYPL1 | NM_006754.5 | ENSG00000008282 | 2783 | 0.478 | 6856 | 61.65 | 91.81 | 98 |
| HPRT1 | NM_000194.3 | ENSG00000165704 | 1612 | 0.445 | 3251 | 45.27 | 34.10 | 98 |
| OTUB1 | NM_017670.3 | ENSG00000167770 | 5336 | 0.586 | 55611 | 32.41 | 55.93 | 95 |
| FEN1 | NM_004111.6 | ENSG00000168496 | 2198 | 0.534 | 2237 | 68.69 | 12.52 | 94 |
| GREG1 | NM_003851.3 | ENSG00000143162 | 3286 | 0.488 | 8804 | 28.39 | 79.76 | 92 |
| CLTB | NM_001834.5 | ENSG00000175416 | 1963 | 0.582 | 1212 | 31.22 | 118.17 | 90 |
| SLC25A1 | NM_005984.5 | ENSG00000100075 | 1999 | 0.677 | 6576 | 36.85 | 69.94 | 89 |
| CIAPIN1 | NM_020313.4 | ENSG00000005194 | 3383 | 0.442 | 57019 | 28.97 | 30.14 | 87 |
| PLTP | NM_006227.4 | ENSG00000100979 | 2541 | 0.543 | 5360 | 18.89 | 87.40 | 86 |
| HMOX2 | NM_002134.4 | ENSG00000103415 | 3377 | 0.558 | 3163 | 18.54 | 29.11 | 84 |
| STX8 | NM_004853.3 | ENSG00000170310 | 3455 | 0.427 | 9482 | 5.11 | 14.86 | 75 |
| CPQ | NM_016134.4 | ENSG00000104324 | 3442 | 0.461 | 10404 | 3.50 | 30.92 | 70 |
| PIK3R3 | NM_003629.4 | ENSG00000117461 | 7397 | 0.430 | 8503 | 6.12 | 14.54 | 64 |
| COMMD3 | NM_012071.4 | ENSG00000148444 | 2903 | 0.367 | 23412 | 20.92 | 33.08 | 57 |
| RRM1 | NM_001033.5 | ENSG00000167325 | 4233 | 0.431 | 6240 | 46.97 | 20.81 | 55 |
| P3H1 | NM_022356.4 | ENSG00000117385 | 6766 | 0.504 | 64175 | 20.68 | 24.62 | 49 |
| HMBS | NM_000190.4 | ENSG00000256269 | 4447 | 0.527 | 3145 | 15.61 | 9.95 | 48 |
| TGIF1 | NM_003244.4 | ENSG00000177426 | 8121 | 0.480 | 7050 | 9.52 | 10.12 | 38 |
| TBP | NM_003194.5 | ENSG00000112592 | 2318 | 0.480 | 6908 | 10.51 | 15.51 | 22 |
| LPCAT4 | NM_153613.3 | ENSG00000176454 | 4702 | 0.510 | 254531 | 21.64 | 35.25 | 20 |
| STYX | NM_145251.4 | ENSG00000198252 | 4983 | 0.364 | 6815 | 6.66 | 16.12 | 16 |
| THG1L | NM_017872.5 | ENSG00000113272 | 2998 | 0.464 | 54974 | 5.41 | 7.69 | 15 |
| USP20 | NM_006676.8 | ENSG00000136878 | 5511 | 0.605 | 10868 | 6.32 | 24.38 | 13 |
| ZSWIM1 | NM_080603.5 | ENSG00000168612 | 2769 | 0.527 | 90204 | 5.33 | 9.41 | 13 |
| PLCB3 | NM_000932.5 | ENSG00000149782 | 4957 | 0.811 | 5331 | 13.99 | 20.57 | 12 |
| SPRTN | NM_032018.7 | ENSG00000010072 | 5906 | 0.423 | 83932 | 4.07 | 3.43 | 12 |
| RASSF4 | NM_032023.4 | ENSG00000107551 | 9481 | 0.548 | 83937 | 3.60 | 37.45 | 10 |
| EGFL8 | NM_030652.4 | ENSG00000241404 | 2609 | 0.563 | 80864 | 14.68 | 50.76 | 9 |
| ATP23 | NM_ 033276.4 | ENSG00000166896 | 3327 | 0.473 | 91419 | 7.30 | 8.77 | 5 |

Design and synthesis of transcript-specific hairpin primers (TSPs), degenerate hairpin primers (DHPs), TaqMan probe, qPCR primers, and synthetic LET-7 miRNAs:

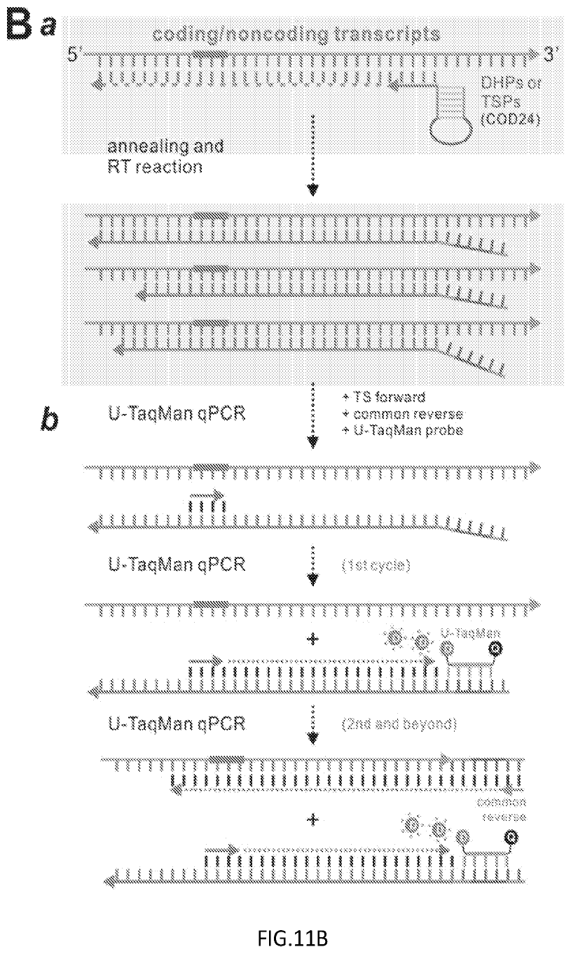

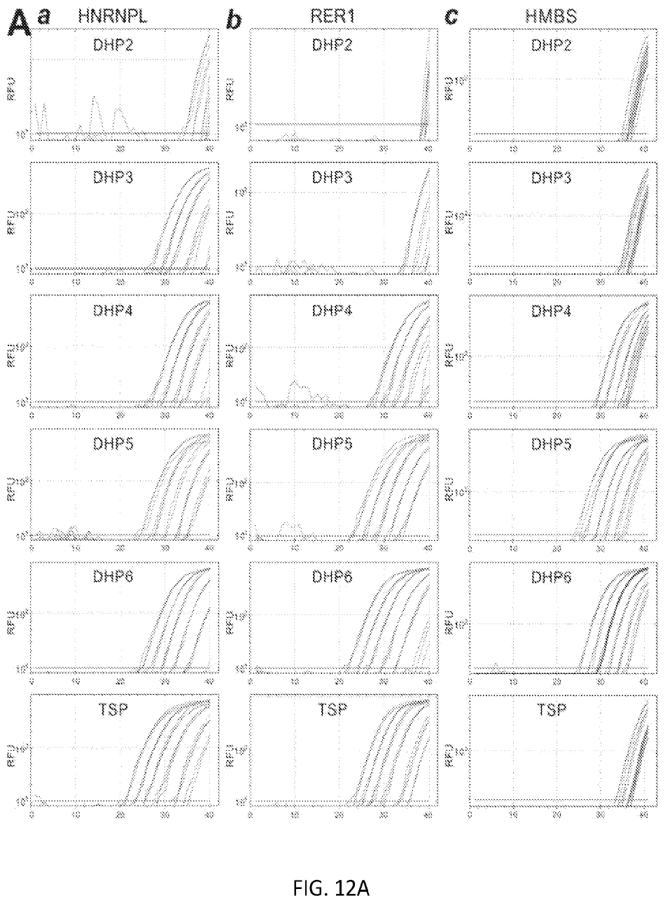

The design of degenerate hairpin primers (DHPs) for reverse transcription (RT) of coding and noncoding transcripts is illustrated in FIG. 11A. Specifically, to generate the DHPs, two, three, four, five or six degenerate, completely randomized nucleotides (nt) were added to the 3′-end of the hairpin core sequence, 5′-GTC GTA TCC AGT GCA GGG TCC GAG GTA TTC GCA CTG GAT ACG AC-3′(SEQ ID NO: 1), yielding DHP2, DHP3, DHP4, DHP5, or DHP6. To make transcript-specific hairpin primers (TSPs) for mRNAs and noncoding RNAs, the corresponding 18-nt reverse primer sequences were added to the 3′-end of the hairpin core sequence, while miRNA-specific hairpin primers were synthesized by adding the last six nucleotides complementary to the 3′-end of mature miRNAs to the 3′-end of the hairpin core sequence. The TaqMan probe cvomprising the nucleotide sequence 5′ CGA ATA CCT CGG ACC CTG CAC-3′ (SEQ ID NO: 30) was synthesized with a FAM dye at the 5′ end, along with a ZEN quencher between CCT and CGG, and an IBFQ quencher at the 3′ end. The TaqMan probe thus comprised the sequence 5′-FAM-CGA ATA CCT (ZEN) CGG ACC CTG CAC-IBFQ-3′. The probe was synthesized by Integrated DNA Technologies (IDT, Coralville, IA).

The qPCR primers were designed by using Primer3Plus, while a common reverse primer 5′-GTA TCC AGT GCA GGG TCC GAG-3′ (SEQ ID NO: 31), was used for universal TaqMan (U-TaqMan) qPCR reactions (see below). The DNA oligonucleotides were synthesized by Millipore Sigma or IDT. Synthetic mature miRNAs for LET-7 family members were obtained from IDT. All oligonucleotide sequences and their utilities are summarized in Table 3.

TABLE 3
List of RT Reaction and qPCR Primers

| Gene/Oligo | | |
|---|---|---|
| Name | Sequence | Use |
| RPS13 TSP | GTCGTATCCAGTGCAGGGTCCGAGGTATTCGCACTGGATACGACTCCAATTGGGAGGGAGGACT (SEQ ID NO: 32) | RT |
| GAPDH TSP | GTCGTATCCAGTGCAGGGTCCGAGGTATTCGCACTGGATACGACAAATGAGCCCCAGCCTTCTC (SEQ ID NO: 33) | |
| HNRNPL TSP | GTCGTATCCAGTGCAGGGTCCGAGGTATTCGCACTGGATACGACCTGGACAGGGCCACAAGG (SEQ ID NO: 34) | |
| HPRT1 TSP | GTCGTATCCAGTGCAGGGTCCGAGGTATTCGCACTGGATACGACATCCAACACTTCGTGGGGTC (SEQ ID NO: 35) | |
| RER1 TSP | GTCGTATCCAGTGCAGGGTCCGAGGTATTCGCACTGGATACGACGACAGGAAACACCGCCCA (SEQ ID NO: 36) | |
| TBP TSP | GTCGTATCCAGTGCAGGGTCCGAGGTATTCGCACTGGATACGACGTGGGGTCAGTCCAGTGC (SEQ ID NO: 37) | |
| HMBS TSP | GTCGTATCCAGTGCAGGGTCCGAGGTATTCGCACTGGATACGACCAAGGCCCCAAGGTGAGG (SEQ ID NO: 38) | |
| PPIA TSP | GTCGTATCCAGTGCAGGGTCCGAGGTATTCGCACTGGATACGACATCCTCCCACGTCAGCCT (SEQ ID NO: 39 | |
| EIF3E TSP | GTCGTATCCAGTGCAGGGTCCGAGGTATTCGCACTGGATACGACAGGAGCCTCTGACCTGCT (SEQ ID NO: 40 | |
| VDAC1 TSP | GTCGTATCCAGTGCAGGGTCCGAGGTATTCGCACTGGATACGACACTGGGGTGGGAACAGGT (SEQ ID NO: 41 | |
| NDUFC1 TSP | GTCGTATCCAGTGCAGGGTCCGAGGTATTCGCACTGGATACGACTGGCCCAGCTCAGTCTCT (SEQ ID NO: 230) | |
| SYPL1 TSP | GTCGTATCCAGTGCAGGGTCCGAGGTATTCGCACTGGATACGACAGGCTGAAGTGCTCACCA (SEQ ID NO: 42) | |
| OTUB1 TSP | GTCGTATCCAGTGCAGGGTCCGAGGTATTCGCACTGGATACGACGGTGGGGCCTATGAGGGA (SEQ ID NO: 43) | |
| FEN1 TSP | GTCGTATCCAGTGCAGGGTCCGAGGTATTCGCACTGGATACGACGACCTTCACCAGCCGCTT (SEQ ID NO: 44) | |
| CREG1 TSP | GTCGTATCCAGTGCAGGGTCCGAGGTATTCGCACTGGATACGACTGCAGGTTGCTCACGGAG (SEQ ID NO: 45) | |
| CLTB TSP | GTCGTATCCAGTGCAGGGTCCGAGGTATTCGCACTGGATACGACTGTTCACTCTGGCGCTGG (SEQ ID NO: 46) | |
| SLC25A1 TSP | GTCGTATCCAGTGCAGGGTCCGAGGTATTCGCACTGGATACGACAAGCGGATGGCCTGGTTC (SEQ ID NO: 47) | |
| CIAPIN1 TSP | GTCGTATCCAGTGCAGGGTCCGAGGTATTCGCACTGGATACGACCAACGATGCCTGGGCTCA (SEQ ID NO: 48) | |
| PLTP TSP | GTCGTATCCAGTGCAGGGTCCGAGGTATTCGCACTGGATACGACCCGCGTGCATTCTGGAGA (SEQ ID NO: 49) | |
| HMOX2 TSP | GTCGTATCCAGTGCAGGGTCCGAGGTATTCGCACTGGATACGACGGGCAAAGGCTGGATGGT (SEQ ID NO: 50) | |
| STX8 TSP | GTCGTATCCAGTGCAGGGTCCGAGGTATTCGCACTGGATACGACGGCCTGCGTCCTGTTCTT (SEQ ID NO: 51) | |
| CPQ TSP | GTCGTATCCAGTGCAGGGTCCGAGGTATTCGCACTGGATACGACTCCCAATGCTGCTGCCAA (SEQ ID NO: 52) | |
| PIK3R3 TSP | GTCGTATCCAGTGCAGGGTCCGAGGTATTCGCACTGGATACGACAAAAGCTCCCACCGCCTC (SEQ ID NO: 53) | |
| COMMD3 TSP | GTCGTATCCAGTGCAGGGTCCGAGGTATTCGCACTGGATACGACTCGGTGCTTTCCTGCCTC (SEQ ID NO: 54) | |
| RRM1 TSP | GTCGTATCCAGTGCAGGGTCCGAGGTATTCGCACTGGATACGACTAGCTGCCAGTAGCCCGA (SEQ ID NO: 55) | |
| P3H1 TSP | GTCGTATCCAGTGCAGGGTCCGAGGTATTCGCACTGGATACGACGTGGGAAGCAAGCTCCGT (SEQ ID NO: 56) | |
| TGIF1 TSP | GTCGTATCCAGTGCAGGGTCCGAGGTATTCGCACTGGATACGACTTTGTCCCACACCGACCG (SEQ ID NO: 57) | |
| LPCAT4 TSP | GTCGTATCCAGTGCAGGGTCCGAGGTATTCGCACTGGATACGACTGTACAGCAGGCGGTTGG (SEQ ID NO: 58) | |
| STYX TSP | GTCGTATCCAGTGCAGGGTCCGAGGTATTCGCACTGGATACGACACTCCTCGGCGTCTTCCT (SEQ ID NO: 59) | |
| THG1L TSP | GTCGTATCCAGTGCAGGGTCCGAGGTATTCGCACTGGATACGACGGGAGGGCTTGCATGGTT (SEQ ID NO: 60) | |
| USP20 TSP | GTCGTATCCAGTGCAGGGTCCGAGGTATTCGCACTGGATACGACGCGAGAGGAGCTGCGTAG (SEQ ID NO: 61) | |
| ZSWIM1 TSP | GTCGTATCCAGTGCAGGGTCCGAGGTATTCGCACTGGATACGACCAATCCAGCTGTCGGCCA (SEQ ID NO: 62) | |
| PLCB3 TSP | GTCGTATCCAGTGCAGGGTCCGAGGTATTCGCACTGGATACGACGCTCGATGAGTAGCGCGT (SEQ ID NO: 63) | |
| SPRTN TSP | GTCGTATCCAGTGCAGGGTCCGAGGTATTCGCACTGGATACGACGCACGCTCCACTTCACCT (SEQ ID NO: 64) | |
| RASSF4 TSP | GTCGTATCCAGTGCAGGGTCCGAGGTATTCGCACTGGATACGACCAGACGCAGGGCTTGGAA (SEQ ID NO: 65) | |
| EGFL8 TSP | GTCGTATCCAGTGCAGGGTCCGAGGTATTCGCACTGGATACGACGGGACCACCAGTGTCTGC (SEQ ID NO: 66) | |
| ATP23 TSP | GTCGTATCCAGTGCAGGGTCCGAGGTATTCGCACTGGATACGACGTGGTGGGTGAGACAGGC (SEQ ID NO: 67) | |
| HSALNT0181736 | GTCGTATCCAGTGCAGGGTCCGAGGTATTCGCACTGGATACGACTGTCTGGCATGGCTGACA (SEQ ID NO: 68) | |
| (TAGLN) TSP | | |
| HSALNT0138467 | GTCGTATCCAGTGCAGGGTCCGAGGTATTCGCACTGGATACGACACAGGCGGGAAATGTGGA (SEQ ID NO: 69) | |
| (TRAM1) TSP | | |
| HSALNT0014988 | GTCGTATCCAGTGCAGGGTCCGAGGTATTCGCACTGGATACGACACGTAGCCAGTCAGCACT (SEQ ID NO: 70) | |
| (MCL1) TSP | | |
| HSALNT0279564 | GTCGTATCCAGTGCAGGGTCCGAGGTATTCGCACTGGATACGACAGGATTGGTGCTGTGGGT (SEQ ID NO: 71) | |
| TSP | | |
| HSALNT0168940 | GTCGTATCCAGTGCAGGGTCCGAGGTATTCGCACTGGATACGACAGTTCGCTGGGCAGACTT (SEQ ID NO: 72) | RT |
| TSP | | |
| HSALNT0016278 | GTCGTATCCAGTGCAGGGTCCGAGGTATTCGCACTGGATACGACAGATGGTGGGTTGCCCTT (SEQ ID NO: 73) | |
| TSP | | |
| HSALNT0289462 | GTCGTATCCAGTGCAGGGTCCGAGGTATTCGCACTGGATACGACAAGGCTGCTCCGTGATGT (SEQ ID NO: 74) | |
| (H19) TSP | | |
| HSALNT0289004 | GTCGTATCCAGTGCAGGGTCCGAGGTATTCGCACTGGATACGACAAAGCGGTGCCACTGTGT (SEQ ID NO: 75) | |
| (HOTAIR) TSP | | |
| HSALNT0289363 | GTCGTATCCAGTGCAGGGTCCGAGGTATTCGCACTGGATACGACGTGTGGTTGCCAAGCCAA (SEQ ID NO: 76) | |
| (MALAT1) TSP | | |
| HSALNT0289343 | GTCGTATCCAGTGCAGGGTCCGAGGTATTCGCACTGGATACGACACAGCTGCGAAGTGCCAT (SEQ ID NO: 77) | |
| (XIST) TSP | | |
| hsa-miR-122-5p | GTCGTATCCAGTGCAGGGTCCGAGGTATTCGCACTGGATACGACCAAACA (SEQ ID NO: 78) | RT |
| TSP | | |
| hsa-miR-4510 | GTCGTATCCAGTGCAGGGTCCGAGGTATTCGCACTGGATACGACAACCAT (SEQ ID NO: 79) | |
| TSP | | |
| hsa-miR-192-3p | GTCGTATCCAGTGCAGGGTCCGAGGTATTCGCACTGGATACGACCTGTGA (SEQ ID NO: 80) | |
| TSP | | |
| hsa-miR-182-5p | GTCGTATCCAGTGCAGGGTCCGAGGTATTCGCACTGGATACGACAGTGTG (SEQ ID NO: 81) | |
| TSP | | |
| hsa-miR-221-5p | GTCGTATCCAGTGCAGGGTCCGAGGTATTCGCACTGGATACGACAAATCT (SEQ ID NO: 82) | |
| TSP | | |
| hsa-miR-215-3p | GTCGTATCCAGTGCAGGGTCCGAGGTATTCGCACTGGATACGACTATTGG (SEQ ID NO: 83) | |
| TSP | | |
| hsa-miR-510-5p | GTCGTATCCAGTGCAGGGTCCGAGGTATTCGCACTGGATACGACGTGATT (SEQ ID NO: 84) | |
| TSP | | |
| hsa-miR-4425 | GTCGTATCCAGTGCAGGGTCCGAGGTATTCGCACTGGATACGACATGGTC (SEQ ID NO: 85) | |
| TSP | | |
| hsa-miR-4672 | GTCGTATCCAGTGCAGGGTCCGAGGTATTCGCACTGGATACGACTGCCTC (SEQ ID NO: 86) | |
| TSP | | |
| hsa-miR-3688-3p | GTCGTATCCAGTGCAGGGTCCGAGGTATTCGCACTGGATACGACAGAGTG (SEQ ID NO: 87) | |
| TSP | | |
| hsa-miR-151a-5p | GTCGTATCCAGTGCAGGGTCCGAGGTATTCGCACTGGATACGACACTAGA (SEQ ID NO: 88) | |
| TSP | | |
| hsa-miR-3688-5p | GTCGTATCCAGTGCAGGGTCCGAGGTATTCGCACTGGATACGACATATGG (SEQ ID NO: 89) | |
| TSP | | |
| hsa-miR-181a-5p | GTCGTATCCAGTGCAGGGTCCGAGGTATTCGCACTGGATACGACACTCAC (SEQ ID NO: 90) | |
| TSP | | |
| hsa-miR-1268a | GTCGTATCCAGTGCAGGGTCCGAGGTATTCGCACTGGATACGACCCCCCA (SEQ ID NO: 91) | |
| TSP | | |
| Common Reverse | GTATCCAGTGCAGGGTCCGAG (SEQ ID NO: 31) | qPCR |
| GAPDH | CAACGAATTTGGCTACAGCA (SEQ ID NO: 92) | qPCR |
| | AGGGGAGATTCAGTGTGGTG (SEQ ID NO: 93) | |
| HPRT1 | TTGCTTTCCTTGGTCAGGCA (SEQ ID NO: 94) | |
| | ATCCAACACTTCGTGGGGTC (SEQ ID NO: 95) | |
| RPS13 | GCAGTTGCTGTTCGAAAGCA (SEQ ID NO: 96) | |
| | TCCAATTGGGAGGGAGGACT (SEQ ID NO: 97) | |
| TBP | GAGTTCCAGCGCAAGGGT (SEQ ID NO: 98) | |
| | GTGGGGTCAGTCCAGTGC (SEQ ID NO: 99) | |
| HNRNPL | CCGGAGCGTGAACAGTGT (SEQ ID NO: 100) | |
| | CTGGACAGGGCCACAAGG (SEQ ID NO: 101) | |
| RER1 | ATGGCAGTCGCTGGACAC (SEQ ID NO: 102) | |
| | GACAGGAAACACCGCCCA (SEQ ID NO: 103) | |
| HMBS | TGCACGGCAGCTTAACGA (SEQ ID NO: 104) | |
| | CAAGGCCCCAAGGTGAGG (SEQ ID NO: 105) | |
| PPIA | GTAGGCAGCAACTGGGCA (SEQ ID NO: 106) | |
| | ATCCTCCCACGTCAGCCT (SEQ ID NO: 107) | |
| EIF3E | CTGTCGCATCCACCAGTGT (SEQ ID NO: 108) | |
| | AGGAGCCTCTGACCTGCT (SEQ ID NO: 109) | |
| VDAC1 | GGAGAACTTGGTGGCCCC (SEQ ID NO: 110) | |
| | ACTGGGGTGGGAACAGGT (SEQ ID NO: 111) | |
| NDUFC1 | GGGCCTGGCTGTCGTATC (SEQ ID NO: 112) | |
| | TGGCCCAGCTCAGTCTCT (SEQ ID NO: 113) | |
| SYPL1 | GCATTGCTGCCCTTCTGC (SEQ ID NO: 114) | |
| | AGGCTGAAGTGCTCACCA (SEQ ID NO: 115) | |
| OTUB1 | CAGGTTTGAGGGGCCAGG (SEQ ID NO: 116) | |
| | GGTGGGGCCTATGAGGGA (SEQ ID NO: 117) | |
| FEN1 | CCGCCACAGCTCAAGTCA (SEQ ID NO: 118) | |
| | GACCTTCACCAGCCGCTT (SEQ ID NO: 119) | |
| CREG1 | CTTCGCCGACGTCCTCTC (SEQ ID NO: 120) | |
| | TGCAGGTTGCTCACGGAG (SEQ ID NO: 121) | |
| CLTB | CCATTGCCCAGGCTGACA (SEQ ID NO: 122) | |
| | TGTTCACTCTGGCGCTGG (SEQ ID NO: 123) | |
| SLC25A1 | GTGTGCCCCATGGAGACC (SEQ ID NO: 124) | |
| | AAGCGGATGGCCTGGTTC (SEQ ID NO: 125) | |
| CIAPIN1 | GAGTGAAGCTGGCTGGGG (SEQ ID NO: 126) | |
| | CAACGATGCCTGGGCTCA (SEQ ID NO: 127) | |
| PLTP | TCCGGAGACAGCTGCTCT (SEQ ID NO: 128) | |
| | CCGCGTGCATTCTGGAGA (SEQ ID NO: 129) | |
| HMOX2 | AAGGAAGCACACGACCGG (SEQ ID NO: 130) | |
| | GGGCAAAGGCTGGATGGT (SEQ ID NO: 131) | |
| STX8 | AGGAGCACCCAACCCTTG (SEQ ID NO: 132) | |
| | GGCCTGCGTCCTGTTCTT (SEQ ID NO: 133) | |
| CPQ | CCTGCAGCAAGATGGGCT (SEQ ID NO: 134) | |
| | TCCCAATGCTGCTGCCAA (SEQ ID NO: 135) | |
| PIK3R3 | CCAGTGGGACCCCGAAAC (SEQ ID NO: 136) | |
| | AAAAGCTCCCACCGCCTC (SEQ ID NO: 137) | |
| COMMD3 | ATCCCCGCTCCTTCGACT (SEQ ID NO: 138) | |
| | TCGGTGCTTTCCTGCCTC (SEQ ID NO: 139) | |
| RRM1 | GGTACCAACCGCCCACAA (SEQ ID NO: 140) | |
| | TAGCTGCCAGTAGCCCGA (SEQ ID NO: 141) | |
| P3H1 | GCGGCGCTGCAAGAATAC (SEQ ID NO: 142) | |
| | GTGGGAAGCAAGCTCCGT (SEQ ID NO: 143) | |
| TGIF1 | CCCTAGGGAGGCCACTGT (SEQ ID NO: 144) | |
| | TTTGTCCCACACCGACCG (SEQ ID NO: 145) | |
| LPCAT4 | TGTGGCCCTTGCACTAGC (SEQ ID NO: 146) | |
| | TGTACAGCAGGCGGTTGG (SEQ ID NO: 147) | |
| STYX | GGCCGGCTGTGTAACACT (SEQ ID NO: 148) | |
| | ACTCCTCGGCGTCTTCCT (SEQ ID NO: 149) | |
| THG1L | GGACAAAGCCAGTGCCCT (SEQ ID NO: 150) | |
| | GGGAGGGCTTGCATGGTT (SEQ ID NO: 151) | |
| USP20 | TGGACACTGCCATGGCTG (SEQ ID NO: 152) | |
| | GCGAGAGGAGCTGCGTAG (SEQ ID NO: 153) | |
| ZSWIM1 | AGCATCCTGGGCAGCAAG (SEQ ID NO: 154) | |
| | CAATCCAGCTGTCGGCCA (SEQ ID NO: 155) | |
| PLCB3 | ACCCACGGCTTCACCATG (SEQ ID NO: 156) | |
| | GCTCGATGAGTAGCGCGT (SEQ ID NO: 157) | |
| SPNRT | CGTGGGAGTTGGTGGACC (SEQ ID NO: 158) | |
| | GCACGCTCCACTTCACCT (SEQ ID NO: 159) | |
| RASSF4 | TGGAAGCTGACTTGGGCG (SEQ ID NO: 160) | |
| | CAGACGCAGGGCTTGGAA (SEQ ID NO: 161) | |
| EGFL8 | AGGGTCCAGCGTCAAAGC (SEQ ID NO: 162) | |
| | GGGACCACCAGTGTCTGC (SEQ ID NO: 163) | |
| ATP23 | CCTCCCGGCCAGAACTTG (SEQ ID NO: 164) | |
| | GTGGTGGGTGAGACAGGC (SEQ ID NO: 165) | |
| Twist1 | CGGCCAGGTACATCGACT (SEQ ID NO: 166) | |
| | CCATCCTCCAGACCGAGA (SEQ ID NO: 167) | |
| Twist1 | GAAAGGAAAGGCATCACTATGG (SEQ ID NO: 168) | |
| | AGACACCGGATCTATTTGCATT (SEQ ID NO: 169) | |
| CTGF | TTGGCCCAGACCCAACTA (SEQ ID NO: 170) | |
| | GCAGGAGGCGTTGTCATT (SEQ ID NO: 171) | |
| S100B | CAGGAATTCATGGCCTTTGT (SEQ ID NO: 172) | |
| | GCTGGAAAGCTCAGCTCCTA (SEQ ID NO: 173) | |
| Cyclin D1/CCND1 | TGTTTGCAAGCAGGACTTTG (SEQ ID NO: 174) | |
| | TGGCACCAAAGGATTCCTAA (SEQ ID NO: 175) | |
| SOX9 | CACCTGTGCCTCTCAGAACA (SEQ ID NO: 176) | |
| | TGAGGAAAGCTCCAACAACC (SEQ ID NO: 177) | |
| C-MYC | CCACCTCCAGCTTGTACCTG (SEQ ID NO: 178) | |
| | GAGCAGAGAATCCGAGGACG (SEQ ID NO: 179) | |
| CYR61/CCN1 | CTGAGTGCCGCCTTGTGA (SEQ ID NO: 180) | |
| | AACCGCAGTACTTGGGCC (SEQ ID NO: 181) | |
| FOXD1 | TGCAGAGCCCCAAGAAGC (SEQ ID NO: 182) | |
| | GAGGTTGTGGCGGATGCT (SEQ ID NO: 183) | |
| PDGFRA | AGGGCCGTGTGACTTTCG (SEQ ID NO: 184) | |
| | AGCAGCCACCGTGAGTTC (SEQ ID NO: 185) | |
| THBS1 | GCCACGGCCAACAAACAG (SEQ ID NO: 186) | |
| | TGTCCTCCCCGCAGATGA (SEQ ID NO: 187) | |
| AXIN2 | GTTCACCCAGGACCCTGC (SEQ ID NO: 188) | |
| | CTGCTTTGGGGGCTTCGA (SEQ ID NO: 189) | |
| AXIN2 | GTGACTTGCCTCCCGGAC (SEQ ID NO: 190) | |
| | CTTCGTTCCGCCTGGTGT (SEQ ID NO: 191) | |
| AXIN2 | GGGGACTCGGGAGCCTAA (SEQ ID NO: 192) | |
| | GTGGACCTCACACTCGCC (SEQ ID NO: 193) | |
| AXIN2 | CGCAGTACCACTCCCTGC (SEQ ID NO: 194) | |
| | CGGCATGGTGGTGGATGT (SEQ ID NO: 195) | |
| AXIN2 | GAACCCTGCTCCTTCGGG (SEQ ID NO: 196) | |
| | AGCACAGCGGCAGTGATT (SEQ ID NO: 197) | |
| AXIN2 | ATCACTGCCGCTGTGCTT (SEQ ID NO: 198) | |
| | GGAAAGGTGCTGCTGGGT (SEQ ID NO: 199) | |
| RUNX2 | CTCCACCCACCCAAGCAG (SEQ ID NO: 200) | |
| | ACGCTGTCCTGCAATGCT (SEQ ID NO: 201) | |
| RUNX2 | AACCGCACCATGGTGGAG (SEQ ID NO: 202) | |
| | CTCCGAGGGCTACCACCT (SEQ ID NO: 203) | |
| RUNX2 | CCCCAGGCAGTTCCCAAG (SEQ ID NO: 204) | |
| | CTTTGGGAAGAGCCGGGG (SEQ ID NO: 205) | |
| RUNX2 | AGCTGTGTATGGACCAGTGC (SEQ ID NO: 206) | |
| | ATGAGGACCTGCAGCATGTC (SEQ ID NO: 207) | |
| RUNX2 | TCCTCTGAAAAGGCAGCAGG (SEQ ID NO: 208) | |
| | GCATGCCACAGAAGGACTCT (SEQ ID NO: 209) | |
| TAGLN | TGCGGCTTTACCCACCTT (SEQ ID NO: 210) | qPCR |
| | TGTCTGGCATGGCTGACA (SEQ ID NO: 211) | |
| TRAM1 | ACAGCTGGCTTACTGGCT (SEQ ID NO: 212) | |
| | ACAGGCGGGAAATGTGGA (SEQ ID NO: 213) | |
| MCL1 | TGGTTTTTAGGGGCCCCA (SEQ ID NO: 214) | |
| | ACGTAGCCAGTCAGCACT (SEQ ID NO: 215) | |
| HSALNT0279564 | AATCCAAGCCTCACCCCA (SEQ ID NO: 216) | |
| | AGGATTGGTGCTGTGGGT (SEQ ID NO: 217) | |
| HSALNT0168940 | AAGATGGCGGCTGCTGTA (SEQ ID NO: 218) | |
| | AGTTCGCTGGGCAGACTT (SEQ ID NO: 219) | |
| HSALNT0016278 | ACATTATCCGGCAGCCCT (SEQ ID NO: 220) | |
| | AGATGGTGGGTTGCCCTT (SEQ ID NO: 221) | |
| H19 | ACGAGTGTGCGTGAGTGT (SEQ ID NO: 222) | |
| | AAGGCTGCTCCGTGATGT (SEQ ID NO: 223) | |
| HOTAIR | GCCTTTGCTTCGTGCTGA (SEQ ID NO: 224) | |
| | AAAGCGGTGCCACTGTGT (SEQ ID NO: 225) | |
| MALATI | TGAGGAGCAAGCGAGCAA (SEQ ID NO: 226) | |
| | GTGTGGTTGCCAAGCCAA (SEQ ID NO: 227) | |
| XIST | TTTTGCCGCCTAGTGCCA (SEQ ID NO: 228) | |
| | ACAGCTGCGAAGTGCCAT (SEQ ID NO: 229) | |
| hsa-miR-122-5p | AGCCTGGAGTGTGACAATGGT (SEQ ID NO: 16) | qPCR |
| Forward | | |
| hsa-miR-4510 | AGCCTGAGGGAGTAGGATGTA (SEQ ID NO: 17) | |
| Forward | | |
| hsa-miR-192-3p | AGCCCTGCCAATTCCATAGGT (SEQ ID NO: 18) | |
| Forward | | |
| hsa-miR-182-5p | AGCCTTTGGCAATGGTAGAAC (SEQ ID NO: 19) | |
| Forward | | |
| hsa-miR-221-5p | AGCCACCTGGCATACAATGTA (SEQ ID NO: 20) | |
| Forward | | |
| hsa-miR-215-3p | AGCCTCTGTCATTTCTTTAGG (SEQ ID NO: 21) | |
| Forward | | |
| hsa-miR-510-5p | AGCCTACTCAGGAGAGTGGCA (SEQ ID NO: 22) | |
| Forward | | |
| hsa-miR-4425 | AGCCTGTTGGGATTCAGCAGG (SEQ ID NO: 23) | |
| Forward | | |
| hsa-miR-4672 | AGCCTTACACAGCTGGACAGA (SEQ ID NO: 24) | |
| Forward | | |
| hsa-miR-3688-3p | AGCCTATGGAAAGACTTTGCC (SEQ ID NO: 25) | |
| Forward | | |
| hsa-miR-151a-5p | AGCCTCGAGGAGCTCACAGTC (SEQ ID NO: 26) | |
| Forward | | |
| hsa-miR-3688-5p | AGCCAGTGGCAAAGTCTTTCC (SEQ ID NO: 27) | |
| Forward | | |
| hsa-miR-181a-5p | AGCCAACATTCAACGCTGTCG (SEQ ID NO: 28) | |
| Forward | | |

| | |
|---|---|
| hsa-miR-1268a | AGCCCGGGCGTGGTGGTGGGG (SEQ ID NO: 29) |
| Forward | |

### Total RNA Isolation and Reverse Transcription (RT) Using TSP, DHP, or Cocktail of DHP (COD) Primers:

Total RNA was isolated from exponentially growing HEK-293, 143B, SJSA1, A375, Mel-624, Mel-888, and/or UC-MSC cells using the NucleoZOL RNA Isolation kit (Takara Bio USA, Mountain View, CA) by following the manufacturer's instructions as described (28-30). For RT reactions, one microgram of total RNA was mixed with 2.0 μg/reaction of the TSP, DHP (i.e., DHP2, DHP3, DHP4, DHP5 and UHP6), or COD primers, and heated at 70° C. for 5 min, followed by cooling down on ice. Each RNA/hairpin primer mixture was added with 2 μl of 10× RT Buffer (NEB), 1 μl of 5 mM dNTPs (GenScript), 0.2 μl of M-MuLV Reverse Transcriptase (NEB), and appropriate volume of RNase-free ddH2O to make up the final volume of 20p. The RT reactions were carried out at 37° C. for 1 h and inactivated at 92° C. for 5 min. Eighty microliters of ddH2O were added to the RT products, which were used as qPCR templates with further dilutions and kept at −80° C. in aliquots.

### Identification of Optimal Cocktails of DHPs (CODs) Through Linear Regression-Based Machine Learning Analysis:

To select potential optimal cocktails of DHPs (CODs) that may yield Cq values closest to those of TSPs, various molar ratios of DHPs were mixed U-TaqMan qPCR reactions were performed, along with respective TSPs, which were subjected to machine learning analysis. Based on the outcomes of machine learning analysis, the mixing and selection processes were repeated multiple cycles until an optimal COD was obtained. Specifically, the Scikit-learn, an open source machine learning Python module integrating a wide range of machine learning algorithms (31), was used. Linear regression model in the python Scikit-learn package was used to identify the best suitable COD (combination of N2, N3, N4, N5, N6). For different CODs, the predicting value using machine learning linear regression (processing methods including LinearRegression, Ridge, Lasso, SGDRegressor) were compared with corresponding “TSP” values, and coefficient of correlation, slope value, intercept value, and p-value for Paired-Samples T Test (using a python SciPy package), were evaluated. For the selected candidate CODs, the composition proportion for every DHP was further adjusted, and then U-TaqMan qPCRs were performed. The CODs were compared with corresponding “TSP” values, and coefficient of correlation, slope value, intercept value, and p-value for paired-samples t-test (using a python SciPy package), were calculated. A p<0.05 was considered statistically significant.

Universal TaqMan (U-TaqMan) Probe-Based qPCR Analysis:

The RT products were further diluted (usually 1:500 to 1:1,000) and used as templates for U-TqMan qPCR analysis. The U-TaqMan qPCR reactions were carried out by using the 2× PrimeTime™ Gene Expression Master Mix (IDT) on a CFX-Connect unit (Bio-Rad Laboratories, Hercules, CA) as previously described (32-34). The qPCR primers for mRNAs, lncRNAs, and miRNAs were designed by using Primer3 Plus, whenever possible, and are listed in Table 3. Briefly, a typical 20 μl qPCR reaction consisted of 10 μl of 2× PrimeTime™ Gene Expression Master Mix, 4 μl of RT templates, 2 μl of transcript-specific forward primers (20 ng/μl stock), 2 μl of common reverse primer (20 ng/μl stock), 2 μl of U-TaqMan probe (final concentration at 100 μM). The qPCR cycling program was as follows: 95° C.×3′ for one cycle; 95° C.×15″, 60° C.×1′, followed by plate read, for 40 cycles. No template control (NTC) was used as negative control. All reactions were done in triplicate. To quantitatively assess the Cq deviation from transcript-specific hairpin primer (TSP) group, ΔCq values were calculated for the DHP or COD groups by subtracting individual average Cq value from corresponding Cq value for the TSP group: ΔCq=average Cq (TSP)−average Cq (DHP or COD).

SYBR Green-Based qPCR Analysis:

Conventional SYBR Green-based touchdown qPCR (TqPCR) analysis was carried out as described (32, 35, 36). Briefly, the RT products were diluted and used as templates. TqPCR reactions were set up by using the 2× Forget-Me-Not™ EvaGreen qPCR Master Mix (Biotium, Fremont, CA), and carried out by using CFX-Connect (Bio-Rad) as previously described (22, 32, 37). TqPCR cycling program was as follows: 95° C.×3′ for one cycle; 95° C.×20″, 66° C.×10″, for 4 cycles by decreasing 3° C. per cycle; 95° C.×20″, 55° C.×10″, 70° cx 1″, followed by plate read, for 40 cycles. Serial dilutions were performed to determine the amplification efficiency for each qPCR primer pairs. No template control (NTC) was used as negative control. All reactions were done in triplicate. To quantitatively assess the Cq deviation from transcript-specific hairpin primer (TSP) group, ΔCq values were calculated for the DHP or COD groups by subtracting individual average Cq value from corresponding Cq value for the TSP group: ΔCq=average Cq (TSP)−average Cq (DHP or COD).

### Data Analysis and Statistical Evaluation:

All qPCR reactions were done in triplicate. The nonparametric Kruskal-Wallis test was carried out to assess the statistical difference among the ΔCq values of the DHPs, relative to that of transcript-specific hairpin (TSP) primer group's. Linear regression and correlation coefficient analysis were also carried out to assess the differences of ΔCq values among groups as described (35, 38, 39). Whenever a comparison was made, a p-value<0.05 was considered statistically significant.

### Results and Discussion

A universal TaqMan (U-TaqMan) probe-based qPCR using degenerate hairpin primers (DHPs) quantifies transcript levels with varied sensitivities and specificities:

To engineer a universal TaqMan probe for qPCR analysis, a panel of degenerate hairpin primers (DHPs) for reverse transcription (RT) reactions was designed (FIG. 11A, panel a). The DHP primers consist of three parts: a stem, a loop, and 2, 3, 4, 5, or 6 randomized nucleotides at the 3′-end of the stem sequence, resulting DHP2, DHP3, DHP4, DHP5 or DHP6 (FIG. 11A, panel a and panel b). As positive controls, 18-nt transcript-specific hairpin primers (TSPs) were added to the 3′-end of the same stem-loop sequence. Mechanistically, the RNA samples (mRNAs and/or noncoding RNAs) were first subjected to RT reactions with DHPs or TSPs, leading to the incorporation of the stem-loop sequence into the 5′-end of the RT products (FIG. 11B, panel a). A universal TaqMan (U-TaqMan) probe, which is complementary to the loop region of DHPs, was designed to quantify the RT products through TaqMan-based qPCR analysis (FIG. 11B, panel b).

To assess which DHPs would quantify gene expression at the sensitivity and specificity close to that of gene-specific TSP primers, a panel of 37 tester genes was selected based on gene expression abundance in human transcriptome (Table 2). The average transcript abundance (either in RPKM or TPM) of human transcriptome was obtained from three RNA-seq datasets: the Broad Institute Cancer Cell Line Encyclopedia (CCLE) RNA-seq dataset (FIG. 16A), the Genotype-Tissue Expression (GTEx) RNA-seq dataset (FIG. 16B), and the homemade CA-14 RNA-seq dataset from 14 human cancer lines (FIG. 16C). The human transcriptomic datasets indicate that approximately 80% of the human genes are expressed at low levels (e.g., <10 RPKM or TPM), while approximately 10-20% are expressed at medium levels (e.g., 10-100 RPKM or TPM), and only <10% of human genes are expressed at high levels (e.g., >100 RPKM or TPM) (FIG. 16). Thus, the selection and composition of the 37 tester genes was based on the distribution of gene expression abundances in order to ensure that the qPCR quantification results were more accurate and generalizable.

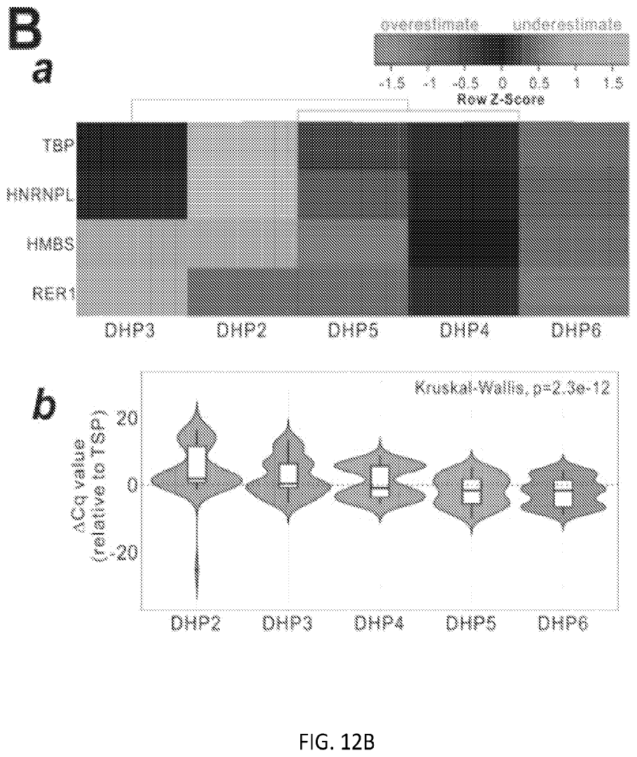

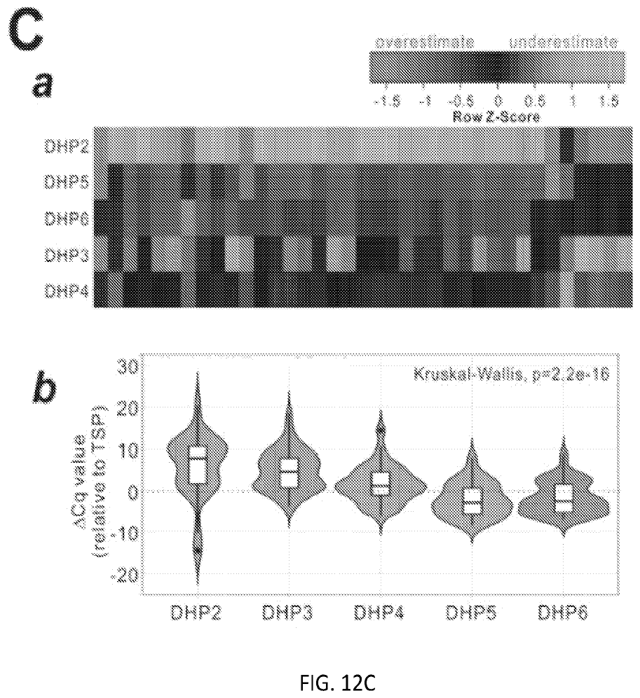

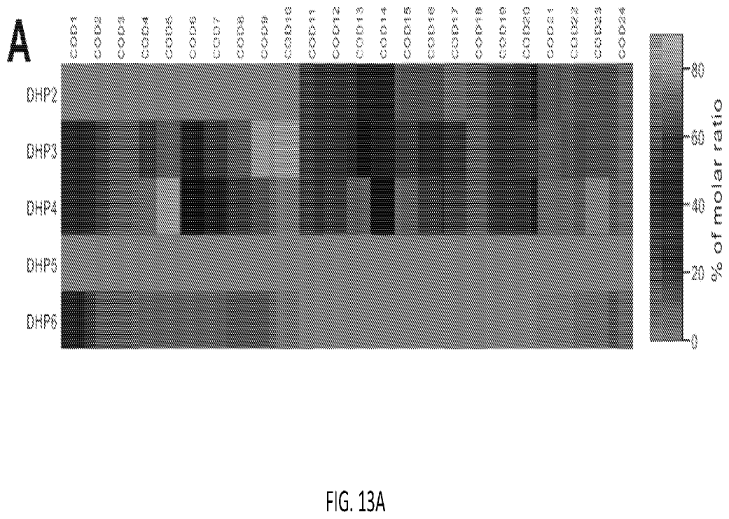

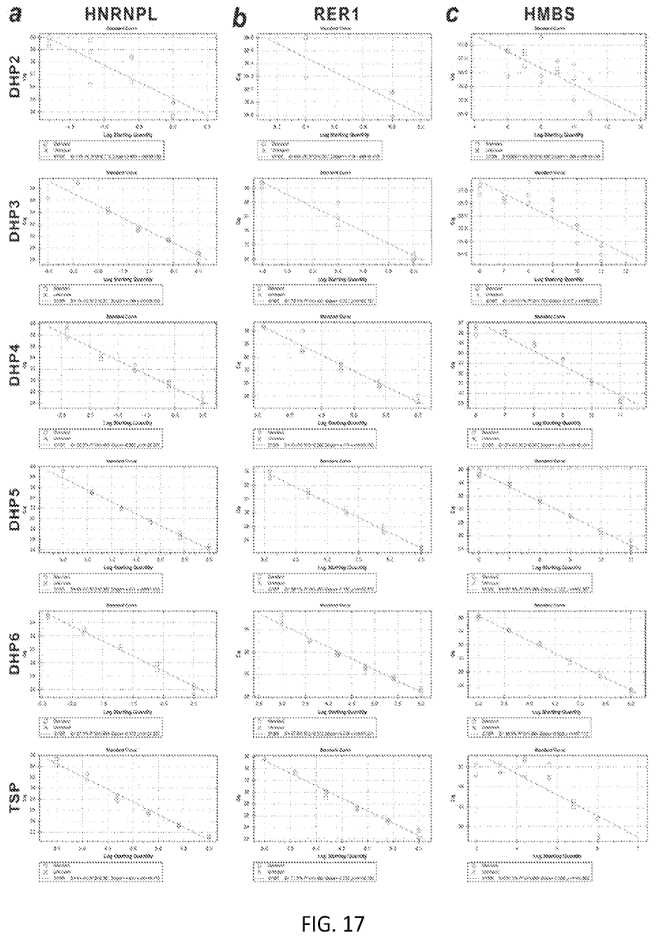

U-TaqMan qPCR quantification outcomes of the 37 tester genes were tested using the five DHPs and their respective TSPs. Three representative transcripts with high (HNRNPL), medium (RER1) and low (HMBS) abundances were shown in FIG. 12A. The five DHP primers were shown to detect HNRNPL expression in HEK293 although DHP2 yielded significantly less efficient amplification while DHP3, DHP4, DHP5 and DHP6 yielded higher Cq values, compared with that of the TSP group (FIG. 12A-a). DHP4, DHP5 and DHP6 were shown to effectively detect RER1 expression, similar to that of RER1's TSP, while DHP2 and DHP3 poorly detected RER1 expression (FIG. 12A-b). For the low abundant transcript HMBS, DHP2 and DHP3 seemingly detected its expression at a similar level to that of HMBS' TSP, while DHP4, DHP5 and DHP6 seemingly overestimated HMBS expression (FIG. 12A-c). The above results are consistent with the standard curves obtained for the five DHP primers and respective TSPs for HNRNPL (FIG. 17a), RER1 (FIG. 17b), and HMBS (FIG. 17c). Collectively, these results indicate that DHP2 and/or DHP3 tended to underestimate expression levels, while DHP5 and/or DHP6 seemingly overestimated transcript expression, compared with that of respective TSP primers.

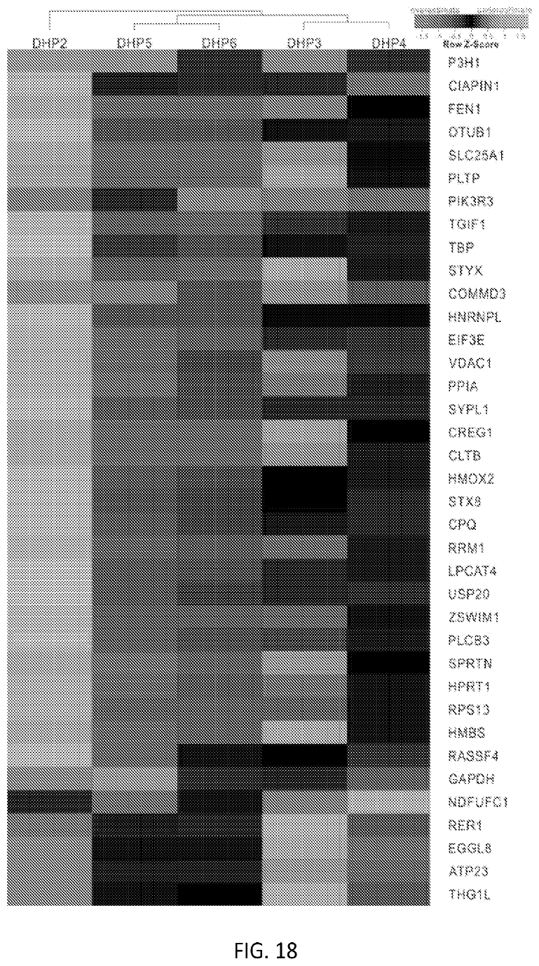

The average ΔCq values were calculated for each DHP primer by subtracting the Cq (TSP) from the Cq (DHPs). The obtained average ΔCq values were subjected to heatmap and clustering analysis. DHP4 primer groups yielded the lowest ΔCq values for the four representative transcripts (TBP, HNRNPL, HMBS and RER1) (FIG. 12B-a), which is also confirmed by the box and whisker plot analysis (FIG. 12B-b). The above heatmap and clustering analysis results of the average ΔCq values are consistently observed for all 37 tester genes with a few exceptions (FIG. 12C-a, & FIG. 18). Furthermore, the box and whisker plot analysis of the 37 tester genes indicates that the average ΔCq values for the DHP4 primer groups were the closest one to zero with relatively small variations, relative to the TSP groups (FIG. 12C-b). The nonparametric Kruskal-Wallis analysis indicates there was a statistical difference among the five DHPs (p value=2.2e-16). Taken together, these results strongly suggest that the DHP4 primer may closely represent the TSPs for most genes, while DHP2 and/or DHP3 may underestimate, and DHP5 and/or DHP6 may overestimate transcript levels in most cases.

### Optimal Cocktails of DHPs (CODs) as Potential Universal TSP Surrogates can be Identified Through Linear Regression-Based Machine Learning Analysis:

Even though the above results demonstrate that DHP4 primer could serve as a TSP surrogate for most transcripts with medium to high abundances, the DHP2 and/or DHP3 primers yielded Cq values similar to that of the TSPs for highly abundant transcripts. On the contrary, the DHP5 and/or DHP6 primers were shown to detect low abundant transcripts more effectively and yielded Cq values close to that of the TSPs. Thus, it is conceivable that optimal cocktail of DHPs (CODs) as potential TSP surrogates may be identified by mixing the DHPs at different molar ratios.

To select optimal CODs, Scikit-learn, an open source machine learning Python module integrating a wide range of machine learning algorithms (_), was used. ΔCq values yielded by individual DHPs were first analyzed for the 37 tester genes for theoretical TSP correlations contributed by individual DHPs in combinations of five, four, three, or two DHPs (assuming at equal molar ratio), or alone. A theoretical combination of DHP2, DHP3, DHP4 and DHP6 yielded the highest coefficient of correlation (R=0.6432) (Table 4), suggesting that potential optimal CODs may be derived from mixtures of these four DHPs.

TABLE 4
Correlation Contributions by Individual DHPs

| Run | Model | R | Slope | Intercept | p value | Weight |
|---|---|---|---|---|---|---|
| 0 | [‘N2’, ‘N3’, ‘N4’, ‘N5’, ‘N6’] | 0.6428 | 0.9084 | 2.3453 | 0.5879 | [−0.04616233 0.18669776 0.03862718 0.02003047 0.75127583] |
| 1 | [‘N2’, ‘N3’, ‘N4’, ‘N5’] | 0.8115 | 0.8819 | 3.1338 | 0.6235 | [−0.0358588 0.24210845 −0.00979176 0.83238151] |
| 2 | [‘N2’, ‘N3’, ‘N4’, ‘N6’] | 0.6432 | 0.9100 | 2.2983 | 0.5695 | [−0.04650911 0.1836939 0.04933072 0.78292562] |
| 3 | [‘N2’, ‘N3’, ‘N5’, ‘N6’] | 0.8413 | 0.9034 | 2.4842 | 0.5817 | [−0.04683561 0.20050344 0.05020337 0.74752687] |
| 4 | [‘N2’, ‘N4’, ‘N5’, ‘N6’] | 0.6428 | 0.9464 | 1.2940 | 0.6196 | [−0.01777246 0.30927473 -0.21449088 0.83367752] |
| 5 | [‘N3’, ‘N4’, ‘N5’, ‘N6’] | 0.6404 | 0.8181 | 2.1568 | 0.5985 | [0.14960446 0.03143901 0.06266114 0.73128286] |
| 6 | [‘N2’, ‘N3’, ‘N4’] | 0.8093 | 0.9529 | 1.1780 | 0.7804 | [−0.05129885 0.113374 0.83277256] |
| 7 | [‘N4’, ‘N5’, ‘N6’] | 0.6415 | 0.9443 | 1.3286 | 0.6220 | [0.28221633 −0.17639334 0.81763826] |
| 8 | [‘N3’, ‘N5’, ‘N6’] | 0.6393 | 0.8110 | 2.2718 | 0.5044 | [0.18080336 0.09352971 0.72841593] |
| 9 | [‘N3’, ‘N4’, ‘N6’] | 0.8419 | 0.9205 | 2.0060 | 0.6060 | [0.13807241 0.0624244 0.76781481] |
| 10 | [‘N3’, ‘N4’, ‘N5’] | 0.6817 | 0.8918 | 2.8477 | 0.5310 | [0.21188423 −0.01908818 0.65288286] |
| 11 | [‘N2’, ‘N5’, ‘N6’] | 0.8178 | 0.8879 | 2.8180 | 0.5581 | [0.02037797 0.13033277 0.87048805] |
| 12 | [‘N2’, ‘N4’, ‘N5’] | 0.6361 | 0.9308 | 1.8004 | 0.6045 | [−0.00624911 0.23719201 0.69734769] |
| 13 | [‘N2’, ‘N4’, ‘N5’] | 0.6081 | 0.9340 | 1.0567 | 0.6702 | [0.0036923 0.34452893 0.40705112] |
| 14 | [‘N2’, ‘N4’, ‘N6’] | 0.6423 | 0.9059 | 2.4125 | 0.5818 | [−0.04777897 0.20291541 0.20905466] |
| 15 | [‘N2’, ‘N3’, ‘N5’] | 0.6118 | 0.8831 | 3.1013 | 0.6262 | [−0.03592485 0.23873898 0.62337845] |
| 16 | [‘N2’, ‘N3’] | 0.5395 | 0.8483 | 1.3748 | 0.8042 | [−0.00088634 0.51016801] |
| 17 | [‘N2’, ‘N4’] | 0.6981 | 0.9720 | 0.8243 | 0.7601 | [−0.02530477 0.62614365] |
| 18 | [‘N2’, ‘N5’] | 0.8803 | 0.8881 | 3.0157 | 0.0008 | [0.04747125 0.82488863] |
| 19 | [‘N2’, ‘N6’] | 0.6185 | 0.9010 | 2.5201 | 0.5581 | [0.01782071 1.01122828] |
| 20 | [‘N3’, ‘N4’] | 0.6015 | 0.9768 | 0.4992 | 0.7792 | [0.08368453 0.55081943] |
| 21 | [‘N3’, ‘N5’] | 0.6122 | 0.8838 | 2.7944 | 0.6338 | [0.20667662 0.63906461] |
| 22 | [‘N3’, ‘N6’] | 0.6405 | 0.9148 | 2.1620 | 0.5986 | [0.18198843 0.82675394] |
| 23 | [‘N4’, ‘N5’] | 0.8081 | 0.9338 | 1.6660 | 0.6888 | [0.35042669 0.40218762] |
| 24 | [‘N2’, ‘N6’] | 0.6361 | 0.3318 | 1.8820 | 0.6065 | [0.23164182 0.7008935] |
| 25 | [‘N2’] | 0.1639 | 0.7088 | 6.7033 | 0.8823 | [0.13085003] |
| 26 | [‘N3’] | 0.5368 | 1.0008 | −0.1579 | 0.8398 | [0.44377645] |
| 27 | [‘N4’] | 0.8089 | 0.9812 | 0.3669 | 0.7722 | [0.81145003] |
| 28 | [‘N5’] | 0.5742 | 0.8723 | 3.3663 | 0.5785 | [0.84672605] |
| 29 | [‘N6’] | 0.6180 | 0.8987 | 2.5870 | 0.5485 | [1.02313437] |

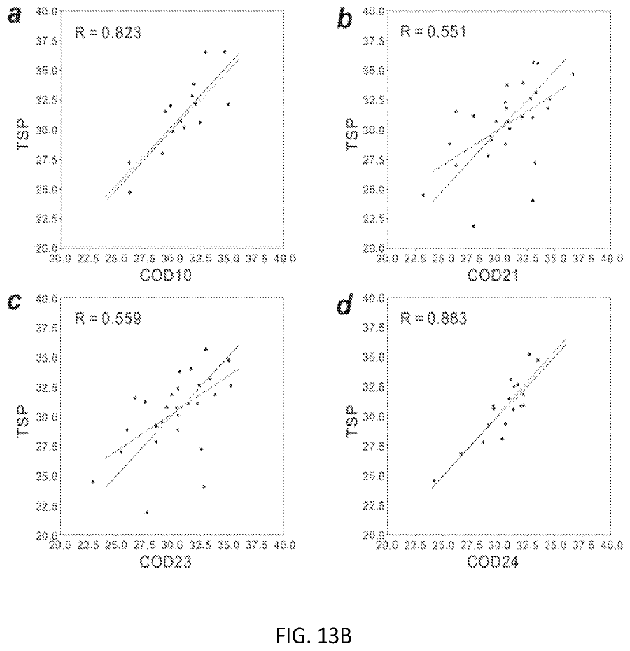

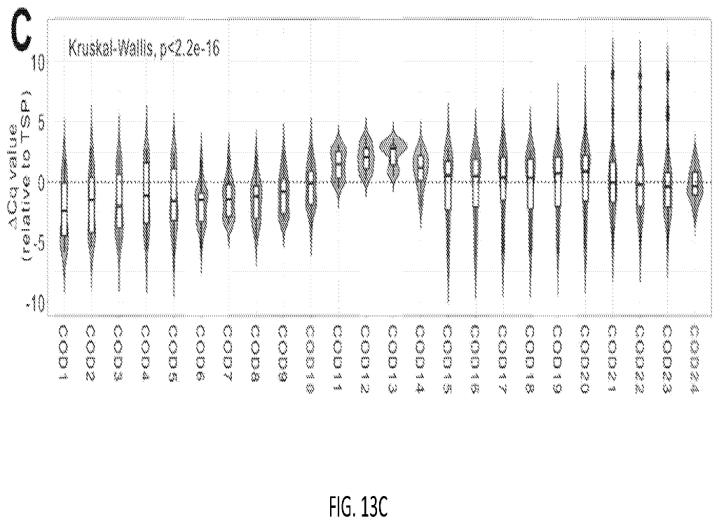

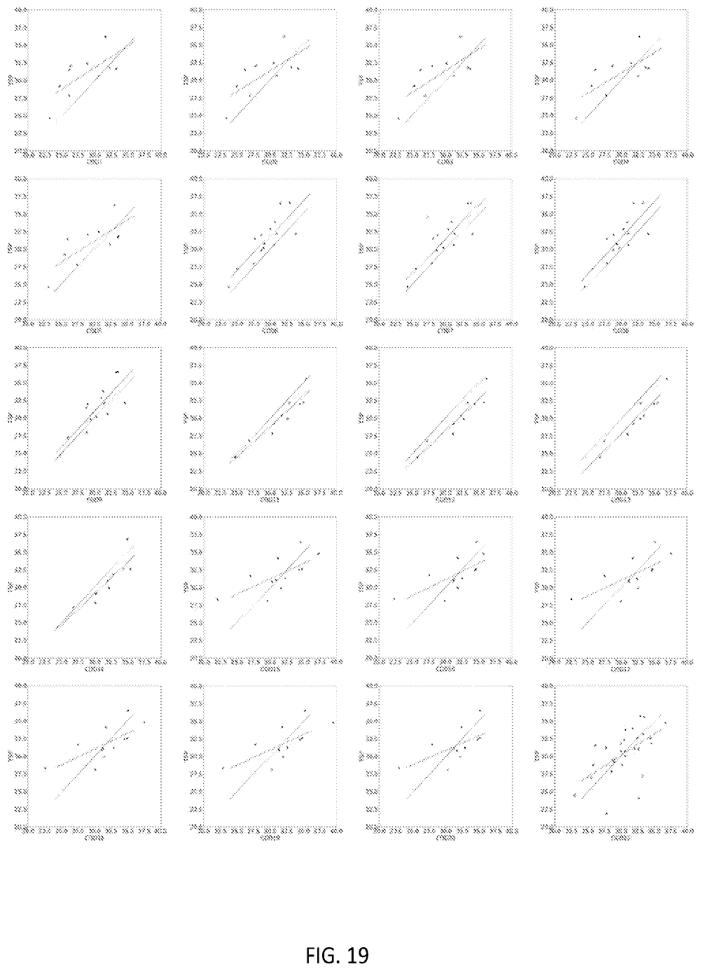

Optimal CODs should have a high coefficient of correlation (R) but no statistical difference when compared with the Cq values of the TSP groups (e.g., in the paired t-test, p>0.05). Six CODs (i.e., COD1 to COD6) were prepared by mixing different molar ratios of DHP2, DHP3, DHP4 and DHP6 (FIG. 13A, & Table 5), and RT reactions and TaqMan qPCR analysis of the tester genes was performed, along with respective TSPs, followed by linear regression model-based machine learning analysis. Based on the obtained correlation values (R) and p values from each round, a total of four rounds of testing (COD1 to COD24) were conducted and the R and p values for each COD (Table 6) were determined. Typical linear regression and correlation plots are shown for COD10, COD21, COD23, and COD24 (FIG. 13B, panels a-d) while the plots for the rest of the 20 CODs are shown in FIG. 19.

TABLE 5
Compositions of the CODs (% molar ratios)

| Test Round | COD | DHP2 | DHP3 | DHP4 | DHP5 | DHP6 |
|---|---|---|---|---|---|---|
| 1st | COD1 | 0 | 33.33 | 33.33 | 0 | 33 |
| | COD2 | 0 | 30 | 50 | 0 | 20 |
| | COD3 | 0 | 20 | 60 | 0 | 20 |
| | COD4 | 0 | 20 | 70 | 0 | 10 |
| | COD5 | 0 | 10 | 80 | 0 | 10 |
| | COD6 | 0 | 50 | 40 | 0 | 10 |
| 2nd | COD7 | 0 | 60 | 30 | 0 | 10 |
| | COD8 | 0 | 70 | 20 | 0 | 10 |
| | COD9 | 0 | 80 | 10 | 0 | 10 |
| | COD10 | 0 | 90 | 5 | 0 | 5 |
| | COD11 | 33.33 | 33.33 | 33.33 | 0 | 0 |
| | COD12 | 50 | 30 | 20 | 0 | 0 |
| 3rd | COD13 | 50 | 40 | 10 | 0 | 0 |
| | COD14 | 30 | 30 | 40 | 0 | 0 |
| | COD15 | 10 | 20 | 70 | 0 | 0 |
| | COD16 | 10 | 30 | 60 | 0 | 0 |
| | COD17 | 10 | 40 | 50 | 0 | 0 |
| | COD18 | 20 | 20 | 60 | 0 | 0 |
| 4th | COD19 | 20 | 30 | 50 | 0 | 0 |
| | COD20 | 30 | 20 | 50 | 0 | 0 |
| | COD21 | 15 | 10 | 70 | 0 | 5 |
| | COD22 | 10 | 15 | 70 | 0 | 5 |
| | COD23 | 10 | 10 | 75 | 0 | 5 |
| | COD24 | 10 | 10 | 70 | 0 | 10 |

TABLE 6
Correlation analysis of the Cocktails of Degenerate hairpins (CODs)
through linear regression-based machine learning analysis

| Mode | R | Slope | Intercept | Paired t-test p-value |
|---|---|---|---|---|
| COD1 | 0.696 | 0.617 | 13.304 | 0.0149537 |
| COD2 | 0.702 | 0.590 | 13.634 | 0.0698081 |
| COD3 | 0.730 | 0.611 | 13.044 | 0.0599270 |
| COD4 | 0.712 | 0.571 | 13.891 | 0.1920094 |
| COD5 | 0.742 | 0.602 | 13.064 | 0.1270415 |
| COD6 | 0.837 | 0.988 | 2.287 | 0.0007268 |
| COD7 | 0.868 | 0.975 | 2.181 | 0.0034115 |
| COD8 | 0.829 | 1.017 | 1.076 | 0.0040724 |
| COD9 | 0.835 | 0.995 | 1.137 | 0.0448110 |
| COD10 | 0.823 | 1.008 | 0.112 | 0.4516049 |
| COD11 | 0.942 | 0.864 | 2.893 | 0.0044727 |
| COD12 | 0.946 | 0.896 | 1.390 | 0.0003212 |
| COD13 | 0.951 | 0.939 | −0.337 | 0.0000472 |
| COD14 | 0.918 | 0.896 | 2.263 | 0.0549853 |
| COD15 | 0.696 | 0.433 | 18.244 | 0.5753530 |
| COD16 | 0.692 | 0.462 | 17.265 | 0.6980543 |
| COD17 | 0.697 | 0.443 | 17.751 | 0.9473489 |
| COD18 | 0.708 | 0.451 | 17.507 | 0.9262894 |
| COD19 | 0.713 | 0.432 | 17.970 | 0.8267940 |
| COD20 | 0.707 | 0.408 | 18.627 | 0.6025178 |
| COD21 | 0.551 | 0.601 | 12.027 | 0.7087992 |
| COD22 | 0.557 | 0.604 | 12.050 | 0.9300716 |
| COD23 | 0.559 | 0.632 | 11.244 | 0.9113338 |
| COD24 | 0.883 | 1.040 | −0.999 | 0.4503574 |

By comparing the predicting values including coefficient of correlation, slope value, intercept value, and p-value from the paired-samples t-test, COD10 and COD24 were found to possibly represent best suitable CODs from multiple rounds of testing since both have high R values and high p values. Nonetheless, box and whisker plot analysis of the ΔCq value relative to TSP for each COD was further conducted, and the nonparametric Kruskal-Wallis test showed statistical differences among the 24 CODs (p<2.2e-16). While the medians for nine CODs (e.g., COD10, COD15, COD16, COD17, COD18, COD21, COD22, COD23 and COD24) were approaching the zero line (i.e., ΔCq=0), only COD24 had the least variability with the smallest upper/lower extreme and quartile ranges (FIG. 13C). Collectively, the linear regression-based machine learning analysis of the 24 CODs identified the COD24 (DHP2:DHP3:DHP4:DHP6=1:1:7:1, molar ratio) as a potential optimal COD and ideal TSP surrogate.

COD24 Functions as a Valid and Reliable TSP Surrogate for TaqMan qPCR Quantification of Gene Expression:

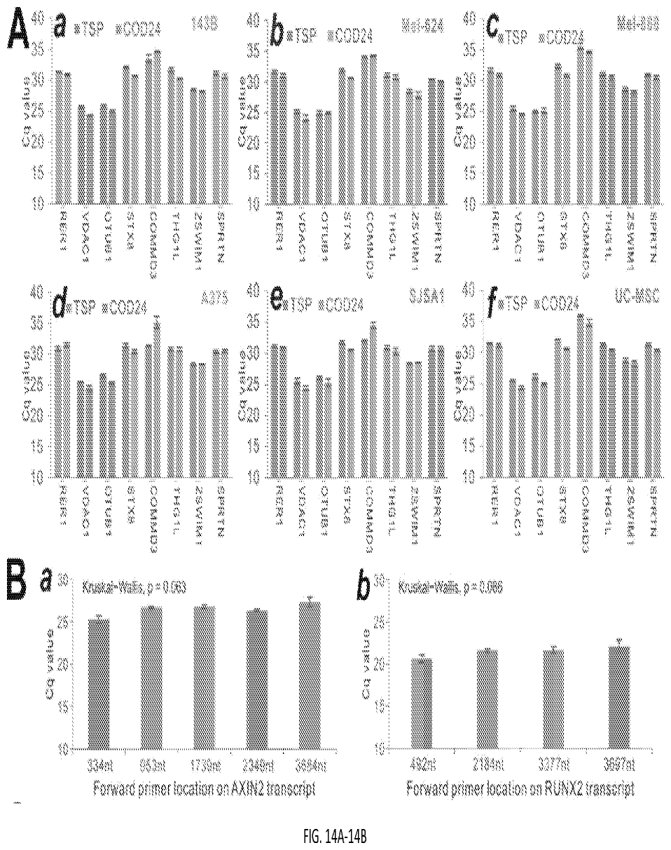

To validate COD24 as a reliable TSP surrogate, total RNA was isolated from six human lines (e.g., 143B, Mel-624, Mel-888, A375, SJSA1 and UC-MSC lines) and COD24-based and TSP-based reverse transcription reactions were performed. Eight tester genes with different abundances were chosen for the validation analysis. The universal TaqMan qPCR reactions were carried out to quantify the expression of eight tester genes. The Cq values obtained from COD24 did not exhibit any statistical differences from those derived from the TSPs of the eight tester genes in all studied six cell lines (FIG. 14A, panels a-f). Thus, these results demonstrate that COD24 functioned as a valid TSP surrogate for RT-qPCR analysis.

Unlike the conventional hexamer-mediated RT-qPCR analysis, in which qPCR quantification is dependent on the use of a pair of transcript-specific forward and reverse primers, the COD24-mediated TaqMan RT-qPCR analysis only requires the use of one transcript-specific forward primer for a given transcript, because the common reverse primer sequence is engineered within the hairpin structure. Thus, it was analyzed whether the locations of transcript-specific forward primers (especially for large transcripts) would affect the Cq values in the COD24-mediated RT-qPCR analysis. Using five forward primers for human AXIN2 transcript (NM_004655, 4,260 nt), the Cq values did not vary significantly among the tested five forward primers (p=0.063) although the 5′-end most forward primer (start site at 334 nt) had a slightly lower Cq value (FIG. 14B-a). Similarly, COD24-mediated RT-qPCR was conducted by using four forward primers for RUNX2 (NM_001024630, 5,540 nt). The differences among their Cq values were not statistically significant (p=0.086) (FIG. 14B-b). Therefore, these results demonstrate that the COD24-mediated RT-qPCR analysis of gene expression is not affected by the location of a transcript-specific forward primer.

The detection sensitivity and specificity between COD24-based TaqMan RT-qPCR and SYBR Green, and/or hexamer-mediated SYBR Green RT-qPCR was next compared. Human UC-MSC cells were infected with adenoviral vector expressing Wnt1, Wnt3, or control GFP (FIG. 14C-a). At 48 h after infection, total RNA were isolated and subjected to COD24 or hexamer-mediated reverse transcription. The expression of the ten well-established target genes of canonical Wnt/β-catenin signaling pathway (40,41) was analyzed with the COD24/TaqMan, COD24/SYBR Green, or hexamer/SYBR Green qPCR systems. Using the COD24/TaqMan system, all ten genes were up-regulated upon Wnt1 and/or Wnt3 stimulation (FIG. 14C-b). However, the SYBR Green qPCR system only detected the upregulation of PDGFRA, while four of the ten target genes were barely detected in the COD24-mediated RT samples (FIG. 14C-c), indicating that the TaqMan qPCR system was more sensitive and efficient than the SYBR Green system. Interestingly, the conventional hexamer/SYBR Green qPCR system only detected three of the ten target genes that were upregulated by Wnt1 and/or Wnt3 (FIG. 14C-d). Taken together, the above results demonstrate that COD24 can function as a valid TSP surrogate and the associated universal TaqMan qPCR system offers sensitive and cost-effective detection of gene expression.

COD24-Based TaqMan qPCR System Quantifies the Expression of lncRNAs and miRNAs with High Specificity:

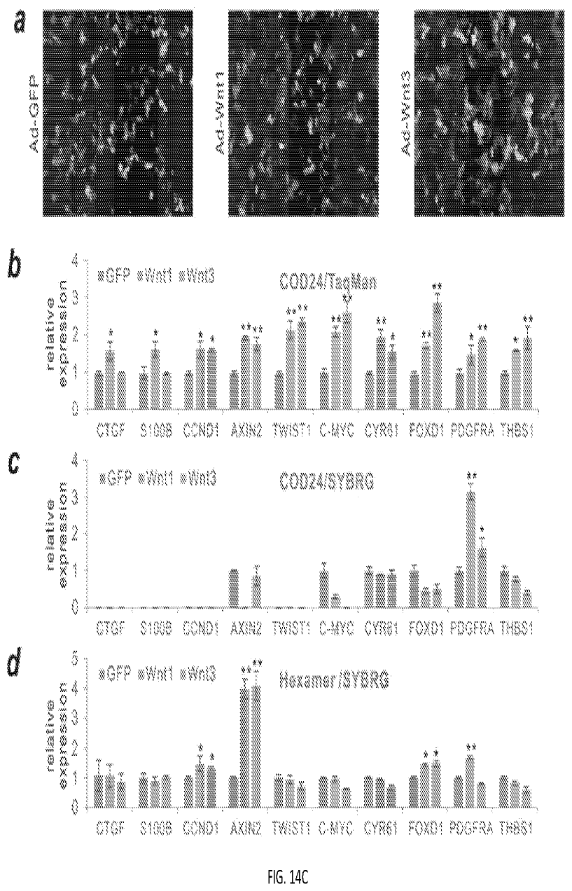

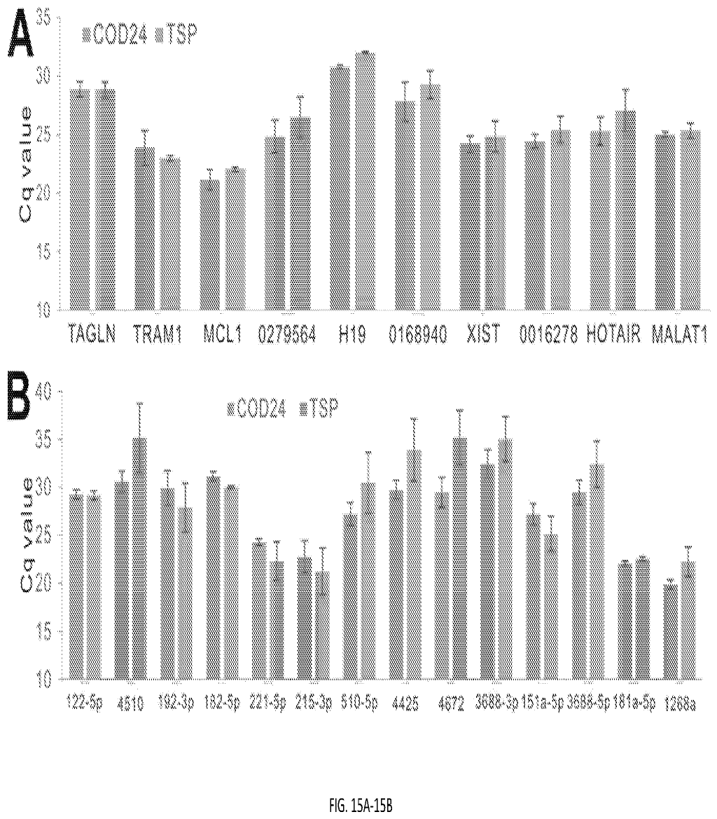

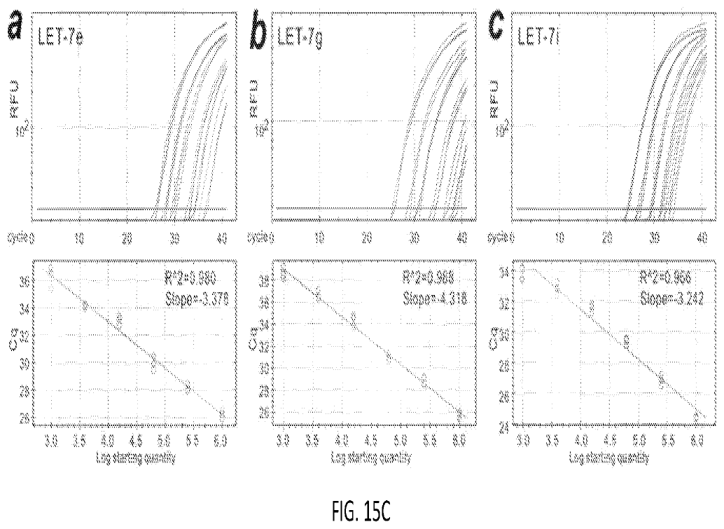

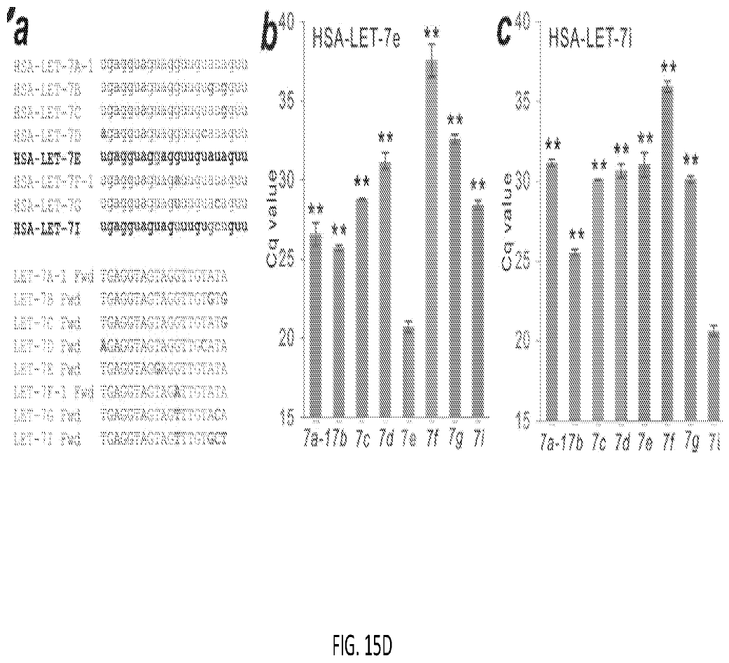

It was further examined whether the COD24-based TaqMan qPCR system could be used to determine the expression of noncoding RNAs such as lncRNAs and miRNAs. Ten commonly studied lncRNAs (Table 7) were selected. RT reactions were cibdycted with either the COD24 primers or lncRNA-specific primers (TSPs), followed by TaqMan qPCR analysis. The Cq values obtained with COD24-based RT-qPCR for the ten lncRNAs were not significantly different from that obtained with TSP-based RT-qPCR (FIG. 15A), indicating that COD24 can be used as a surrogate for lncRNA-specific primers.

Similarly, 14 miRNAs with various expression levels were chosen (Table 7) and their expression levels were analyzed with the COD24 or mature miRNA transcript-specific primers (TSPs) as previously described (38). The Cq values between the COD24 and TSP groups were not statistically different (FIG. 15B). To test the forward primers could discriminate different members of isomiRNA family, the synthetic mature LET-7e, LET-7g and LET-7i were used, and COD24-based TaqMan qPCR quantification of miRNA LET-7 isomiRNA family members was analyzed. Let LET-7 isomiRNA-specific forward primers yielded broad amplification arranges and standard curves with high coefficients of correlation for LET-7e, LET-7g and LET-7i (FIG. 15C, panels a-c). Furthermore, the forward primers for the eight LET-7 family members (FIG. 15D-a) were designed and their qPCR detection specificity was tested on the synthetic LET-7e and LET-7i mature miRNAs. The LET-7e specific forward primer yielded an average Cq value of 20.74, which is at least 5 cycles lower than any of the other seven forward primers of the LET-7 family members (26.59 for LET-7a; 25.71 for LET-7b; 28.80 for LET-7c; 31.21 for LET-7d; 37.58 for LET-7f, 32.62 for LET-7g; and 28.43 for LET-7i) (FIG. 5D-b). Similarly, the LET-7i specific forward primer yielded an average Cq value of 20.67, which is also at least 5 cycles lower than any of the other seven forward primers of the LET-7 family members (31.19 for LET-7a; 25.57 for LET-7b; 30.11 for LET-7c; 30.69 for LET-7d; 31.01 for LET-7e; 35.97 for LET-7f, and 30.15 for LET-7g) (FIG. 15D-c), indicating that miRNA-specific forward primers can detect respective mature miRNA expression with high specificity. Collectively, the above results demonstrate that COD24-based TaqMan qPCR can reliably quantify the expression of both lncRNAs and miRNAs.

TABLE 7
List of the Selected Tester lncRNAs and miRNAs

| Accession | Symbol | Other ID |
|---|---|---|
| HSALNT0181736 | TAGLN | NONHSAT159729.1 |
| HSALNT0138467 | TRAM1 | NONHSAT217194.1 |
| HSALNT0014988 | MCL1 | NONHSAT006313.2 |
| HSALNT0279564 | HSALNT0279564 | NONHSAT222363.1 |
| HSALNT0168940 | HSALNT0168940 | NONHSAT157586.1 |
| HSALNT0016278 | HSALNT0016278 | NONHSAT149935.1 |
| HSALNT0289462 | H19 | NR_002196.2 |
| HSALNT0289004 | HOTAIR | NR_003716.3 |
| HSALNT0289363 | MALAT1 | NR_002819.4 |
| HSALNT0289343 | XIST | NR_001564.2 |
| MIMAT0000421 | HSA-MIR-122-5P | hsa-miR-122-5p |
| MIMAT0019047 | HSA-MIR-4510 | hsa-miR-4510 |
| MIMAT0004543 | HSA-MIR-192-3P | hsa-miR-192-3p |
| MIMAT0000259 | HSA-MIR-182-5P | hsa-miR-182-5p |
| MIMAT0004568 | HSA-MIR-221-5P | hsa-miR-221-5p |
| MIMAT0026476 | HSA-MIR-215-3P | hsa-miR-215-3p |
| MIMAT0002882 | HSA-MIR-510-5P | hsa-miR-510-5p |
| MIMAT0018940 | HSA-MIR-4425 | hsa-miR-4425 |
| MIMAT0019754 | HSA-MIR-4672 | hsa-miR-4672 |
| MIMAT0018116 | HSA-MIR-3688-3P | hsa-miR-3688-3p |
| MIMAT0004697 | HSA-MIR-151A-5P | hsa-miR-151a-5p |
| MIMAT0019223 | HSA-MIR-3688-5P | hsa-miR-3688-5p |
| MIMAT0000256 | HSA-MIR-181A-5P | hsa-miR-181a-5p |
| MIMAT0005922 | HSA-MIR-1268A | hsa-miR-1268a |

COD24-based UniQE system unifies the quantification of expression of both coding and noncoding RNAs using a universal and cost-effective TaqMan RT-qPCR system:

Even though RT-qPCR is one of the most commonly used methods to quantify transcript levels, it has been known the sensitivity, efficiency, and reproducibility of qPCR results vary significantly among different commercial kits (especially for SYBR Green-based kits) (42). TaqMan qPCR analysis represents one of the most sensitive and specific RT-qPCR assays to quantify RNA levels. However, the high cost associated with the synthesis of a TaqMan probe prevents the broad use of transcript-specific TaqMan qPCR analysis. Here, through a linear regression-based machine learning analysis, the UniQE system was established to quantify transcript levels with a universal TaqMan probe in a cost-effective fashion. The UniQE system provides several advantages. First, the optimal cocktail of degenerate hairpin oligonucleotides, COD24, was identified, which serves as the best surrogate to transcript-specific primers and hence removes the necessity of using transcript-specific primers for RT reactions. Second, the use of degenerate hairpin primers reduces non-specific priming and self-priming, compared with degenerate linear primers (38, 43, 44). Third, a common reverse primer sequence and a TaqMan probe recognition (complementary) sequence are built in the hairpin structure so that a common TaqMan probe can be used to drastically reduce the cost associated with probe synthesis, while the use of the common reverse primer further simplify qPCR assays as only transcript-specific forward primers are required. Lastly, the UniQE system was used to quantify both coding (mRNA) and noncoding (lncRNA and miRNA) transcripts with high sensitivity and specificity.

Random hexamers have never been thoroughly analyzed for how faithfully they represent transcript-specific primers (TSPs) in generating reverse transcription products. In fact, it has been well documented that the priming of random hexamers in cDNA synthesis shows sequence bias and in some cases affects sequence coverage uniformity (48-51). While many factors can cause the variations of hexamer priming bias, it is conceivable that transcript abundance may profoundly affect hexamer-priming efficiency. In the experiments herein, a large panel of tester transcripts (genes) closely representing the abundance distribution of human transcriptome was selected to evaluate the TSP representativeness of the five degenerate hairpin primers, DHP2 to DHP6. Surprisingly, while shorter DHPs (i.e., DHP2) tended to underestimate low abundance transcripts, and longer DHPs (i.e., DHP6) seemed to overestimate high abundance transcripts, DHP4 yielded the closest TSP representativeness for the transcripts of broad range of abundances. Machine learning analysis identified the optimal cocktail of degenerate hairpins COD24 (DHP2:DHP3:DPH4:DHP6=1:1:7:1 molar ratio) as a reliable TSP surrogate for both coding and noncoding transcripts.

While in this study TaqMan-based qPCR analysis was employed, the UniQE system can be readily adapted for other forms of fluorophore probe-based qPCR detection chemistry (16). In general, the fluorescent probe molecules used in qPCR reactions can be divided into three groups: 1) primer-based probes, such as Scorpions, Amplifluor, LUX, Cyclicons and Angler; 2) hydrolysis or hybridization-based probes, such as TaaMan, MGB-TaaMan, Snake assay, Hybprobe or FRET, Molecular Beacons, HyBeacon, MGB-Pleiades, MGB-Eclipse and ResonSense; and 3) nucleic acid analogue-based, such as PNA, LNA, ZNA, Plexor primer, and Tiny-Molecular Beacon (16). With some minor changes of sequence design in the hairpin structure of the COD24, the reported UniQE system can be restructured for RT-qPCR analysis using Molecular Beacon or MGB probes. Nonetheless, TaqMan probes, to a less extent Molecular Beacon probes, are among the most commonly used qPCR detection chemistries.

In summary, using the linear regression-based machine learning analysis, a universal TaqMan-based qPCR system (i.e., UniQE) was developed to quantify coding and noncoding RNAs. By carrying out side-by-side comparison with TSPs, five DHPs were tested (designated as DHP2, DHP3, DHP4, DHP5 and DHP6), which share the same hairpin and a sequence complementary to a universal TaqMan (U-TaqMan) probe as TSPs, but contain 2, 3, 4, 5, and 6 randomized anchored nucleotides at the 3′-end of the stem sequence. When the five DHP-mediated RT products for the 37 tester genes were subjected to U-TaqMan qPCR, DHP4 yielded quantification results closest to that of TSPs, whereas DHP6 overestimated and DHP2 underestimated the expression levels of the tester genes, suggesting a possibility to develop an optimal CODs as a reliable surrogate of TSPs for RNA quantification. Through four rounds of linear regression-based machine learning analyses of 24 CODs, the optimal DHP mix (designated as COD24) was identified, which best recapitulated the TSPs in mRNA quantification. The COD24-mediated U-TaqMan qPCR system reliably quantified the expression levels of lncRNAs and miRNAs with high sensitivity and specificity. Collectively, these findings demonstrate that the reported UniQE universal TaqMan qPCR system provides a cost-effective method for coding and noncoding transcriptomic quantification, has broad applications in basic and translational research, as well as in clinical diagnostics.

### REFERENCES

1. Djebali, S., Davis, C. A., Merkel, A., Dobin, A., Lassmann, T., Mortazavi, A., Tanzer, A., Lagarde, J., Lin, W., Schlesinger, F. et al. (2012) Landscape of transcription in human cells. Nature, 489, 101-108.
2. Pertea, M. (2012) The human transcriptome: an unfinished story. Genes (Basel), 3, 344-360.
3. Cech, T. R. and Steitz, J. A. (2014) The noncoding RNA revolution-trashing old rules to forge new ones. Cell, 157, 77-94.
4. Wang, Z., Gerstein, M. and Snyder, M. (2009) RNA-Seq: a revolutionary tool for transcriptomics. Nat Rev Genet, 10, 57-63.
5. Iyer, M. K., Niknafs, Y. S., Malik, R., Singhal, U., Sahu, A., Hosono, Y., Barrette, T. R., Prensner, J. R., Evans, J. R., Zhao, S. et al. (2015) The landscape of long noncoding RNAs in the human transcriptome. Nat Genet, 47, 199-208.
6. Jacquier, A. (2009) The complex eukaryotic transcriptome: unexpected pervasive transcription and novel small RNAs. NatRev Genet, 10, 833-844.
7. Josefsen, K. and Nielsen, H. (2011) Northern blotting analysis. Methods Mol Biol, 703, 87-105.
8. Levy, S. E. and Myers, R. M. (2016) Advancements in Next-Generation Sequencing. Annu Rev Genomics Hum Genet, 17, 95-115.
9. Goodwin, S., McPherson, J. D. and McCombie, W. R. (2016) Coming of age: ten years of next-generation sequencing technologies. Nat Rev Genet, 17, 333-351.
10. Slatko, B. E., Gardner, A. F. and Ausubel, F. M. (2018) Overview of Next-Generation Sequencing Technologies. Curr Protoc Mol Biol, 122, e59.
11. Liu, Q., Wang, Z., Jiang, Y., Shao, F., Ma, Y., Zhu, M., Luo, Q., Bi, Y., Cao, L., Peng, L. et al. (2022) Single-cell landscape analysis reveals distinct regression trajectories and novel prognostic biomarkers in primary neuroblastoma. Genes & Diseases.
12. Vandesompele, J., De Preter, K., Pattyn, F., Poppe, B., Van Roy, N., De Paepe, A. and Speleman, F. (2002) Accurate normalization of real-time quantitative RT-PCR data by geometric averaging of multiple internal control genes. Genome Biol, 3, RESEARCH0034.
13. Bustin, S. and Nolan, T. (2017) Talking the talk, but not walking the walk: RT-qPCR as a paradigm for the lack of reproducibility in molecular research. Eur J Cin Invest, 47, 756-774.
14. Taylor, S. C., Nadeau, K., Abbasi, M., Lachance, C., Nguyen, M. and Fenrich, J. (2019) The Ultimate qPCR Experiment: Producing Publication Quality, Reproducible Data the First Time. Trends Biotechnol, 37, 761-774.
15. Bustin, S. A., Benes, V., Garson, J. A., Hellemans, J., Huggett, J., Kubista, M., Mueller, R., Nolan, T., Pfaffl, M. W., Shipley, G. L. et al. (2009) The MIQE guidelines: minimum information for publication of quantitative real-time PCR experiments. Clin Chem, 55, 611-622.
16. Navarro, E., Serrano-Heras, G., Castano, M. J. and Solera, J. (2015) Real-time PCR detection chemistry. Clin Chim Acta, 439, 231-250.
17. Wu, N., Zhang, H., Deng, F., Li, R., Zhang, W., Chen, X., Wen, S., Wang, N., Zhang, J., Yin, L. et al. (2014) Overexpression of Ad5 precursor terminal protein accelerates recombinant adenovirus packaging and amplification in HEK-293 packaging cells. Gene Ther, 21, 629-637.
18. Wei, Q., Fan, J., Liao, J., Zou, Y., Song, D., Liu, J., Cui, J., Liu, F., Ma, C., Hu, X. et al. (2017) Engineering the Rapid Adenovirus Production and Amplification (RAPA) Cell Line to Expedite the Generation of Recombinant Adenoviruses. Cell Physiol Biochem, 41, 2383-2398.
19. Shu, Y., Yang, C., Ji, X., Zhang, L., Bi, Y., Yang, K., Gong, M., Liu, X., Guo, Q., Su, Y. et al. (2018) Reversibly immortalized human umbilical cord-derived mesenchymal stem cells (UC-MSCs) are responsive to BMP9-induced osteogenic and adipogenic differentiation. J Cell Biochem, 119, 8872-8886.
20. Wu, X., Li, Z., Zhang, H., He, F., Qiao, M., Luo, H., Zhang, J., Zhang, M., Mao, Y., Wagstaff, W. et al. (2021) Modeling colorectal tumorigenesis using the organoids derived from conditionally immortalized mouse intestinal crypt cells (ciMICs). Genes Dis, 8, 814-826.
21. Mao, Y., Ni, N., Huang, L., Fan, J., Wang, H., He, F., Liu, Q., Shi, D., Fu, K., Pakvasa, M. et al. (2021) Argonaute (AGO) proteins play an essential role in mediating BMP9-induced osteogenic signaling in mesenchymal stem cells (MSCs). Genes Dis, 8, 918-930.
22. Huang, B., Huang, L. F., Zhao, L., Zeng, Z., Wang, X., Cao, D., Yang, L., Ye, Z., Chen, X., Liu, B. et al. (2020) Microvesicles (MIVs) secreted from adipose-derived stem cells (ADSCs) contain multiple microRNAs and promote the migration and invasion of endothelial cells. Genes Dis, 7, 225-234.
23. Wang, X., Zhao, L., Wu, X., Luo, H., Wu, D., Zhang, M., Zhang, J., Pakvasa, M., Wagstaff, W., He, F. et al. (2021) Development of a simplified and inexpensive RNA depletion method for plasmid DNA purification using size selection magnetic beads (SSMBs). Genes Dis, 8, 298-306.
24. He, T. C., Zhou, S., da Costa, L. T., Yu, J., Kinzler, K. W. and Vogelstein, B. (1998) A simplified system for generating recombinant adenoviruses. Proc NatlAcad Sci USA, 95, 2509-2514.
25. Lee, C. S., Bishop, E. S., Zhang, R., Yu, X., Farina, E. M., Yan, S., Zhao, C., Zheng, Z., Shu, Y., Wu, X. et al. (2017) Adenovirus-Mediated Gene Delivery: Potential Applications for Gene and Cell-Based Therapies in the New Era of Personalized Medicine. Genes Dis, 4, 43-63.
26. Ni, N., Deng, F., He, F., Wang, H., Shi, D., Liao, J., Zou, Y., Wang, H., Zhao, P., Hu, X. et al. (2021) A one-step construction of adenovirus (OSCA) system using the Gibson DNA Assembly technology. Mol Ther Oncolytics, 23, 602-611.
27. Zhao, C., Wu, N., Deng, F., Zhang, H., Wang, N., Zhang, W., Chen, X., Wen, S., Zhang, J., Yin, L. et al. (2014) Adenovirus-mediated gene transfer in mesenchymal stem cells can be significantly enhanced by the cationic polymer polybrene. PLoS One, 9, e92908.
28. Liu, W., Deng, Z., Zeng, Z., Fan, J., Feng, Y., Wang, X., Cao, D., Zhang, B., Yang, L., Liu, B. et al. (2020) Highly expressed BMP9/GDF2 in postnatal mouse liver and lungs may account for its pleiotropic effects on stem cell differentiation, angiogenesis, tumor growth and metabolism. Genes Dis, 7, 235-244.
29. An, L., Shi, Q., Zhu, Y., Wang, H., Peng, Q., Wu, J., Cheng, Y., Zhang, W., Yi, Y., Bao, Z. et al. (2021) Bone morphogenetic protein 4 (BMP4) promotes hepatic glycogen accumulation and reduces glucose level in hepatocytes through mTORC2 signaling pathway. Genes Dis, 8, 531-544.
30. Zhong, J., Wang, H., Yang, K., Wang, H., Duan, C., Ni, N., An, L., Luo, Y., Zhao, P., Gou, Y. et al. (2022) Reversibly immortalized keratinocytes (iKera) facilitate re-epithelization and skin wound healing: Potential applications in cell-based skin tissue engineering. Bioact Mater, 9, 523-540.
31. Pedregosa, F., Varoquaux, G., Gramfort, A., Michel, V., Thirion, B., Grisel, O., Blondel, M., Prettenhofer, P., Weiss, R., Dubourg, V. et al. (2011) Scikit-learn: Machine Learning in Python. Journal ofMachine Learning Research, 12, 2825-2830.
32. Zhang, Q., Wang, J., Deng, F., Yan, Z., Xia, Y., Wang, Z., Ye, J., Deng, Y., Zhang, Z., Qiao, M. et al. (2015) TqPCR: A Touchdown qPCR Assay with Significantly Improved Detection Sensitivity and Amplification Efficiency of SYBR Green qPCR. PLoS One, 10, e0132666.
33. Zhong, J., Kang, Q., Cao, Y., He, B., Zhao, P., Gou, Y., Luo, Y., He, T. C. and Fan, J. (2021) BMP4 augments the survival of hepatocellular carcinoma (HCC) cells under hypoxia and hypoglycemia conditions by promoting the glycolysis pathway. Am JCancer Res, 11, 793-811.
34. Gou, Y., Weng, Y., Chen, Q., Wu, J., Wang, H., Zhong, J., Bi, Y., Cao, D., Zhao, P., Dong, X. et al. (2022) Carboxymethyl chitosan prolongs adenovirus-mediated expression of IL--10 and ameliorates hepatic fibrosis in a mouse model. Bioengineering & Translational Medicine, n/a, e10306.
35. Fan, J., Wei, Q., Liao, J., Zou, Y., Song, D., Xiong, D., Ma, C., Hu, X., Qu, X., Chen, L. et al. (2017) Noncanonical Wnt signaling plays an important role in modulating canonical Wnt-regulated stemness, proliferation and terminal differentiation of hepatic progenitors. Oncotarget, 8, 27105-27119.
36. Liao, J., Wei, Q., Fan, J., Zou, Y., Song, D., Liu, J., Liu, F., Ma, C., Hu, X., Li, L. et al. (2017) Characterization of retroviral infectivity and superinfection resistance during retrovirus-mediated transduction of mammalian cells. Gene Ther, 24, 333-341.
37. Sun, Y., He, Y., Tong, J., Liu, D., Zhang, H., He, T. and Bi, Y. (2022) All-trans retinoic acid inhibits the malignant behaviors of hepatocarcinoma cells by regulating ferroptosis. Genes & Diseases.
38. He, F., Ni, N., Wang, H., Zeng, Z., Zhao, P., Shi, D., Xia, Y., Chen, C., Hu, D. A., Qin, K. H. et al. (2022) OUHP: an optimized universal hairpin primer system for cost-effective and high-throughput RT-qPCR-based quantification of microRNA (miRNA) expression. Nucleic Acids Res, 50, e22.
39. Fan, J., Feng, Y., Zhang, R., Zhang, W., Shu, Y., Zeng, Z., Huang, S., Zhang, L., Huang, B., Wu, D. et al. (2020) A simplified system for the effective expression and delivery of functional mature microRNAs in mammalian cells. Cancer Gene Ther, 27, 424-437.
40. Yang, K., Wang, X., Zhang, H., Wang, Z., Nan, G., Li, Y., Zhang, F., Mohammed, M. K., Haydon, R. C., Luu, H. H. et al. (2016) The evolving roles of canonical WNT signaling in stem cells and tumorigenesis: implications in targeted cancer therapies. Lab Invest, 96, 116-136.
41. Zhang, F., Song, J., Zhang, H., Huang, E., Song, D., Tollemar, V., Wang, J., Wang, J., Mohammed, M., Wei, Q. et al. (2016) Wnt and BMP Signaling Crosstalk in Regulating Dental Stem Cells: Implications in Dental Tissue Engineering. Genes Dis, 3, 263-276.
42. Sieber, M. W., Recknagel, P., Glaser, F., Witte, O. W., Bauer, M., Claus, R. A. and Frahm, C. (2010) Substantial performance discrepancies among commercially available kits for reverse transcription quantitative polymerase chain reaction: a systematic comparative investigator-driven approach. AnalBiochem, 401, 303-311.
43. Chen, C., Ridzon, D. A., Broomer, A. J., Zhou, Z., Lee, D. H., Nguyen, J. T., Barbisin, M., Xu, N. L., Mahuvakar, V. R., Andersen, M. R. et al. (2005) Real-time quantification of microRNAs by stem-loop RT-PCR. Nucleic Acids Res, 33, e179.
44. Jung, U., Jiang, X., Kaufmann, S. H. and Patzel, V. (2013) A universal TaqMan-based RT-PCR protocol for cost-efficient detection of small noncoding RNA. RNA, 19, 1864-1873.
45. Feinberg, A. P. and Vogelstein, B. (1983) A technique for radiolabeling DNA restriction endonuclease fragments to high specific activity. AnalBiochem, 132, 6-13.
46. Froussard, P. (1992) A random-PCR method (rPCR) to construct whole cDNA library from low amounts of RNA. Nucleic Acids Res, 20, 2900.
47. Zeng, Z., Huang, B., Wang, X., Fan, J., Zhang, B., Yang, L., Feng, Y., Wu, X., Luo, H., Zhang, J. et al. (2020) A reverse transcriptase-mediated ribosomal RNA depletion (RTR2D) strategy for the cost-effective construction of RNA sequencing libraries. JAdv Res, 24, 239-250.
48. Mortazavi, A., Williams, B. A., McCue, K., Schaeffer, L. and Wold, B. (2008) Mapping and quantifying mammalian transcriptomes by RNA-Seq. Nat Methods, 5, 621-628.
49. Hansen, K. D., Brenner, S. E. and Dudoit, S. (2010) Biases in Illumina transcriptome sequencing caused by random hexamer priming. Nucleic Acids Res, 38, e131.
50. Roberts, A., Trapnell, C., Donaghey, J., Rinn, J. L. and Pachter, L. (2011) Improving RNA-Seq expression estimates by correcting for fragment bias. Genome Biol, 12, R22.
51. van Gurp, T. P., McIntyre, L. M. and Verhoeven, K. J. (2013) Consistent errors in first strand cDNA due to random hexamer mispriming. PLoS One, 8, e85583.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A primer nucleic acid molecule comprising a stem-loop structure and a degenerate nucleic acid sequence of 2-10 nucleotides at the 3′ end, wherein the degenerate nucleic acid sequence hybridizes to the 3′-end of a ribonucleic acid (RNA) molecule.

2. The primer nucleic acid molecule of claim 1, wherein the RNA molecule is a mature microRNA (miRNA), a messenger RNA (mRNA), or a long noncoding RNA (lncRNA) molecule.

3. The primer nucleic acid molecule of claim 1 or claim 2, wherein the degenerate nucleic acid sequence comprises 2, 3, 4, or 6 nucleotides.

4. The primer nucleic acid molecule of claim 3, wherein the degenerate nucleic acid sequence comprises 4 nucleotides.

5. The primer nucleic acid molecule of any one of claims 1-4, wherein the stem comprises 14 base pairs and the loop comprises 16 nucleotides.

6. A composition comprising a mixture of two or more primer nucleic acid molecules, wherein each primer nucleic acid molecule comprises a stem-loop structure and a degenerate nucleic acid sequence of 2-10 nucleotides at the 3′ end, wherein the degenerate nucleic acid sequence hybridizes to the 3′-end of a ribonucleic acid (RNA) molecule.

7. The composition of claim 6, wherein the RNA molecule is a mature microRNA (miRNA), a messenger RNA (mRNA), or a long noncoding RNA (lncRNA) molecule.

8. The composition of claim 6 or claim 7, wherein the degenerate nucleic acid sequence comprises 2, 3, 4, or 6 nucleotides.

9. The composition of claim 8, which comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and/or (iii) one or more primer nucleic acid molecules having a generate nucleic acid sequence of 6 nucleotides.

10. The composition of claim 9, comprising (i) one or more primer nucleic acid sequences molecules having a degenerate nucleic acid sequence of 2 nucleotides, (ii) one or more primer nucleic acid molecules having a degenerate nucleic acid sequence of 4 nucleotides, and (iii) one or more primer nucleic acid molecules having a generate nucleic acid sequence of 6 nucleotides.

11. The composition of claim 10, wherein the RNA molecule is a mature miRNA molecule, and wherein the ratio of (i), (ii), and (iii) in the composition is about 8:1:1.

12. The composition of claim 8, comprising (i) one or more primer nucleic acid sequences molecules having a degenerate nucleic acid sequence of 2 nucleotides, (ii) one or more primer nucleic acid molecules having a degenerate nucleic acid sequence of 3 nucleotides, (iii) one or more primer nucleic acid molecules having a generate nucleic acid sequence of 4 nucleotides, and (iv) one or more primer nucleic acid molecules having a generate nucleic acid sequence of 6 nucleotides.

13. The composition of claim 12, wherein the RNA molecule is an miRNA molecule, an mRNA molecule, or a lncRNA molecule, and wherein the molar ratio of (i), (ii), (iii), and (iv) in the composition is about 1:1:7:1.

14. A system for quantifying ribonucleic acid (RNA) in a sample, which comprises:
(a) a primer nucleic acid molecule, or a mixture of primer nucleic acid molecules, wherein the primer nucleic acid molecule or each primer nucleic acid molecule comprises a stem-loop structure and a degenerate nucleic acid sequence of 2-10 nucleotides at the 3′ end, wherein the degenerate nucleic acid sequence hybridizes to the 3′-end of an RNA molecule;
(b) a reverse transcriptase; and
(c) deoxyribonucleotide triphosphates (dNTPs).

15. The system of claim 14, wherein the RNA molecule is a mature microRNA (miRNA), a messenger RNA (mRNA), or a long noncoding RNA (lncRNA) molecule.

16. The system of claim 14 or claim 15, wherein the degenerate nucleic acid sequence comprises 2, 3, 4, or 6 nucleotides.

17. The system of any one of claims 14-16, wherein the stem comprises 14 base pairs and the loop comprises 6 nucleotides.

18. The system of any one of claims 14-17, comprising a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and/or (iii) one or more primer nucleic acid molecules having a generate nucleic acid sequence of 6 nucleotides.

19. The system of claim 18, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 4 nucleotides, and (iii) one or more primer nucleic acid molecules having a generate nucleic acid sequence of 6 nucleotides.

20. The system of claim 19, wherein the RNA molecule is a mature miRNA molecule, and wherein the molar ratio of (i), (ii), and (iii) in the composition is about 8:1:1.

21. The system of any one of claims 14-17, comprising a mixture of primer nucleic acid molecules, wherein the mixture of primer nucleic acid molecules comprises (i) one or more primer nucleic acid sequences molecules having a degenerate nucleic acid sequence of 2 nucleotides, (ii) one or more primer nucleic acid molecules having a degenerate nucleic acid sequence of 3 nucleotides, (iii) one or more primer nucleic acid molecules having a generate nucleic acid sequence of 4 nucleotides, and (iv) one or more primer nucleic acid molecules having a generate nucleic acid sequence of 6 nucleotides.

22. The system of claim 21, wherein the RNA molecule is a mature microRNA (miRNA), a messenger RNA (mRNA), or a long noncoding RNA (lncRNA) molecule and wherein the molar ratio of (i), (ii), (iii), and (iv) in the composition is about 1:1:7:1.

23. A method of quantifying micro RNA (miRNA) in a sample, which comprises
(a) contacting the sample with the system of any one of claims 14-22 under conditions whereby the primer nucleic acid molecule or mixture of primer nucleic acid molecules hybridizes to miRNA present in the sample and reverse transcription of the miRNA occurs; and
(b) amplifying and quantifying the reverse transcribed miRNA using quantitative real-time PCR.

24. A method of quantifying micro RNA (miRNA), long noncoding RNA (lncRNA), and/or messenger RNA (mRNA) in a sample, which comprises
(a) contacting the sample with the system of claim 21 or claim 22 under conditions whereby the mixture of primer nucleic acid molecules hybridizes to miRNA, lncRNA, and/or mRNA present in the sample and reverse transcription of the miRNA, lncRNA, and/or mRNA occurs; and
(b) amplifying and quantifying the reverse transcribed miRNA, lncRNA, and/or mRNA.

25. The method of claim 23 or claim 24, wherein the sample is a biological sample.

26. The method of claim 25, wherein the biological sample comprises mammalian cells.

27. The method of claim 26, wherein the mammalian cells are human cells.


SEQUENCE LISTING (230 sequences)

SEQ ID NO: 1 - DNA, 44 bp, synthetic construct
  source 1..44: mol_type = other DNA
gtcgtatccagtgcagggtccgaggtattcgcactggatacgac

SEQ ID NO: 2 - DNA, 50 bp, synthetic construct
  source 1..50: mol_type = other DNA
gtcgtatccagtgcagggtccgaggtattcgcactggatacgaccaaaca

SEQ ID NO: 3 - DNA, 50 bp, synthetic construct
  source 1..50: mol_type = other DNA
gtcgtatccagtgcagggtccgaggtattcgcactggatacgacaaccat

SEQ ID NO: 4 - DNA, 50 bp, synthetic construct
  source 1..50: mol_type = other DNA
gtcgtatccagtgcagggtccgaggtattcgcactggatacgacctgtga

SEQ ID NO: 5 - DNA, 50 bp, synthetic construct
  source 1..50: mol_type = other DNA
gtcgtatccagtgcagggtccgaggtattcgcactggatacgacagtgtg

SEQ ID NO: 6 - DNA, 50 bp, synthetic construct
  source 1..50: mol_type = other DNA
gtcgtatccagtgcagggtccgaggtattcgcactggatacgacaaatct

SEQ ID NO: 7 - DNA, 50 bp, synthetic construct
  source 1..50: mol_type = other DNA
gtcgtatccagtgcagggtccgaggtattcgcactggatacgactattgg

SEQ ID NO: 8 - DNA, 50 bp, synthetic construct
  source 1..50: mol_type = other DNA
gtcgtatccagtgcagggtccgaggtattcgcactggatacgacgtgatt

SEQ ID NO: 9 - DNA, 50 bp, synthetic construct
  source 1..50: mol_type = other DNA
gtcgtatccagtgcagggtccgaggtattcgcactggatacgacatggtc

SEQ ID NO: 10 - DNA, 50 bp, synthetic construct
  source 1..50: mol_type = other DNA
gtcgtatccagtgcagggtccgaggtattcgcactggatacgactgcctc

SEQ ID NO: 11 - DNA, 50 bp, synthetic construct
  source 1..50: mol_type = other DNA
gtcgtatccagtgcagggtccgaggtattcgcactggatacgacagagtg

SEQ ID NO: 12 - DNA, 50 bp, synthetic construct
  source 1..50: mol_type = other DNA
gtcgtatccagtgcagggtccgaggtattcgcactggatacgacactaga

SEQ ID NO: 13 - DNA, 50 bp, synthetic construct
  source 1..50: mol_type = other DNA
gtcgtatccagtgcagggtccgaggtattcgcactggatacgacatatgg

SEQ ID NO: 14 - DNA, 50 bp, synthetic construct
  source 1..50: mol_type = other DNA
gtcgtatccagtgcagggtccgaggtattcgcactggatacgacactcac

SEQ ID NO: 15 - DNA, 50 bp, synthetic construct
  source 1..50: mol_type = other DNA
gtcgtatccagtgcagggtccgaggtattcgcactggatacgacccccca

SEQ ID NO: 16 - DNA, 21 bp, synthetic construct
  source 1..21: mol_type = other DNA
agcctggagtgtgacaatggt

SEQ ID NO: 17 - DNA, 21 bp, synthetic construct
  source 1..21: mol_type = other DNA
agcctgagggagtaggatgta

SEQ ID NO: 18 - DNA, 21 bp, synthetic construct
  source 1..21: mol_type = other DNA
agccctgccaattccataggt

SEQ ID NO: 19 - DNA, 21 bp, synthetic construct
  source 1..21: mol_type = other DNA
agcctttggcaatggtagaac

SEQ ID NO: 20 - DNA, 21 bp, synthetic construct
  source 1..21: mol_type = other DNA
agccacctggcatacaatgta

SEQ ID NO: 21 - DNA, 21 bp, synthetic construct
  source 1..21: mol_type = other DNA
agcctctgtcatttctttagg

SEQ ID NO: 22 - DNA, 21 bp, synthetic construct
  source 1..21: mol_type = other DNA
agcctactcaggagagtggca

SEQ ID NO: 23 - DNA, 21 bp, synthetic construct
  source 1..21: mol_type = other DNA
agcctgttgggattcagcagg

SEQ ID NO: 24 - DNA, 21 bp, synthetic construct
  source 1..21: mol_type = other DNA
agccttacacagctggacaga

SEQ ID NO: 25 - DNA, 21 bp, synthetic construct
  source 1..21: mol_type = other DNA
agcctatggaaagactttgcc

SEQ ID NO: 26 - DNA, 21 bp, synthetic construct
  source 1..21: mol_type = other DNA
agcctcgaggagctcacagtc

SEQ ID NO: 27 - DNA, 21 bp, synthetic construct
  source 1..21: mol_type = other DNA
agccagtggcaaagtctttcc

SEQ ID NO: 28 - DNA, 21 bp, synthetic construct
  source 1..21: mol_type = other DNA
agccaacattcaacgctgtcg

SEQ ID NO: 29 - DNA, 21 bp, synthetic construct
  source 1..21: mol_type = other DNA
agcccgggcgtggtggtgggg

SEQ ID NO: 30 - DNA, 21 bp, synthetic construct
  source 1..21: mol_type = other DNA
cgaatacctcggaccctgcac

SEQ ID NO: 31 - DNA, 21 bp, synthetic construct
  source 1..21: mol_type = other DNA
gtatccagtgcagggtccgag

SEQ ID NO: 32 - DNA, 64 bp, synthetic construct
  source 1..64: mol_type = other DNA
gtcgtatccagtgcagggtccgaggtattcgcactggatacgactccaattgggagggaggact

SEQ ID NO: 33 - DNA, 64 bp, synthetic construct
  source 1..64: mol_type = other DNA
gtcgtatccagtgcagggtccgaggtattcgcactggatacgacaaatgagccccagccttctc

SEQ ID NO: 34 - DNA, 62 bp, synthetic construct
  source 1..62: mol_type = other DNA
gtcgtatccagtgcagggtccgaggtattcgcactggatacgacctggacagggccacaagg

SEQ ID NO: 35 - DNA, 64 bp, synthetic construct
  source 1..64: mol_type = other DNA
gtcgtatccagtgcagggtccgaggtattcgcactggatacgacatccaacacttcgtggggtc

SEQ ID NO: 36 - DNA, 62 bp, synthetic construct
  source 1..62: mol_type = other DNA
gtcgtatccagtgcagggtccgaggtattcgcactggatacgacgacaggaaacaccgccca

SEQ ID NO: 37 - DNA, 62 bp, synthetic construct
  source 1..62: mol_type = other DNA
gtcgtatccagtgcagggtccgaggtattcgcactggatacgacgtggggtcagtccagtgc

SEQ ID NO: 38 - DNA, 62 bp, synthetic construct
  source 1..62: mol_type = other DNA
gtcgtatccagtgcagggtccgaggtattcgcactggatacgaccaaggccccaaggtgagg

SEQ ID NO: 39 - DNA, 62 bp, synthetic construct
  source 1..62: mol_type = other DNA
gtcgtatccagtgcagggtccgaggtattcgcactggatacgacatcctcccacgtcagcct

SEQ ID NO: 40 - DNA, 62 bp, synthetic construct
  source 1..62: mol_type = other DNA
gtcgtatccagtgcagggtccgaggtattcgcactggatacgacaggagcctctgacctgct

SEQ ID NO: 41 - DNA, 62 bp, synthetic construct
  source 1..62: mol_type = other DNA
gtcgtatccagtgcagggtccgaggtattcgcactggatacgacactggggtgggaacaggt

SEQ ID NO: 42 - DNA, 62 bp, synthetic construct
  source 1..62: mol_type = other DNA
gtcgtatccagtgcagggtccgaggtattcgcactggatacgacaggctgaagtgctcacca

SEQ ID NO: 43 - DNA, 62 bp, synthetic construct
  source 1..62: mol_type = other DNA
gtcgtatccagtgcagggtccgaggtattcgcactggatacgacggtggggcctatgaggga

SEQ ID NO: 44 - DNA, 62 bp, synthetic construct
  source 1..62: mol_type = other DNA
gtcgtatccagtgcagggtccgaggtattcgcactggatacgacgaccttcaccagccgctt

SEQ ID NO: 45 - DNA, 62 bp, synthetic construct
  source 1..62: mol_type = other DNA
gtcgtatccagtgcagggtccgaggtattcgcactggatacgactgcaggttgctcacggag

SEQ ID NO: 46 - DNA, 62 bp, synthetic construct
  source 1..62: mol_type = other DNA
gtcgtatccagtgcagggtccgaggtattcgcactggatacgactgttcactctggcgctgg

SEQ ID NO: 47 - DNA, 62 bp, synthetic construct
  source 1..62: mol_type = other DNA
gtcgtatccagtgcagggtccgaggtattcgcactggatacgacaagcggatggcctggttc

SEQ ID NO: 48 - DNA, 62 bp, synthetic construct
  source 1..62: mol_type = other DNA
gtcgtatccagtgcagggtccgaggtattcgcactggatacgaccaacgatgcctgggctca

SEQ ID NO: 49 - DNA, 62 bp, synthetic construct
  source 1..62: mol_type = other DNA
gtcgtatccagtgcagggtccgaggtattcgcactggatacgacccgcgtgcattctggaga

SEQ ID NO: 50 - DNA, 62 bp, synthetic construct
  source 1..62: mol_type = other DNA
gtcgtatccagtgcagggtccgaggtattcgcactggatacgacgggcaaaggctggatggt

SEQ ID NO: 51 - DNA, 62 bp, synthetic construct
  source 1..62: mol_type = other DNA
gtcgtatccagtgcagggtccgaggtattcgcactggatacgacggcctgcgtcctgttctt

SEQ ID NO: 52 - DNA, 62 bp, synthetic construct
  source 1..62: mol_type = other DNA
gtcgtatccagtgcagggtccgaggtattcgcactggatacgactcccaatgctgctgccaa

SEQ ID NO: 53 - DNA, 62 bp, synthetic construct
  source 1..62: mol_type = other DNA
gtcgtatccagtgcagggtccgaggtattcgcactggatacgacaaaagctcccaccgcctc

SEQ ID NO: 54 - DNA, 62 bp, synthetic construct
  source 1..62: mol_type = other DNA
gtcgtatccagtgcagggtccgaggtattcgcactggatacgactcggtgctttcctgcctc

SEQ ID NO: 55 - DNA, 62 bp, synthetic construct
  source 1..62: mol_type = other DNA
gtcgtatccagtgcagggtccgaggtattcgcactggatacgactagctgccagtagcccga

SEQ ID NO: 56 - DNA, 62 bp, synthetic construct
  source 1..62: mol_type = other DNA
gtcgtatccagtgcagggtccgaggtattcgcactggatacgacgtgggaagcaagctccgt

SEQ ID NO: 57 - DNA, 62 bp, synthetic construct
  source 1..62: mol_type = other DNA
gtcgtatccagtgcagggtccgaggtattcgcactggatacgactttgtcccacaccgaccg

SEQ ID NO: 58 - DNA, 62 bp, synthetic construct
  source 1..62: mol_type = other DNA
gtcgtatccagtgcagggtccgaggtattcgcactggatacgactgtacagcaggcggttgg

SEQ ID NO: 59 - DNA, 62 bp, synthetic construct
  source 1..62: mol_type = other DNA
gtcgtatccagtgcagggtccgaggtattcgcactggatacgacactcctcggcgtcttcct

SEQ ID NO: 60 - DNA, 62 bp, synthetic construct
  source 1..62: mol_type = other DNA
gtcgtatccagtgcagggtccgaggtattcgcactggatacgacgggagggcttgcatggtt

SEQ ID NO: 61 - DNA, 62 bp, synthetic construct
  source 1..62: mol_type = other DNA
gtcgtatccagtgcagggtccgaggtattcgcactggatacgacgcgagaggagctgcgtag

SEQ ID NO: 62 - DNA, 62 bp, synthetic construct
  source 1..62: mol_type = other DNA
gtcgtatccagtgcagggtccgaggtattcgcactggatacgaccaatccagctgtcggcca

SEQ ID NO: 63 - DNA, 62 bp, synthetic construct
  source 1..62: mol_type = other DNA
gtcgtatccagtgcagggtccgaggtattcgcactggatacgacgctcgatgagtagcgcgt

SEQ ID NO: 64 - DNA, 62 bp, synthetic construct
  source 1..62: mol_type = other DNA
gtcgtatccagtgcagggtccgaggtattcgcactggatacgacgcacgctccacttcacct

SEQ ID NO: 65 - DNA, 62 bp, synthetic construct
  source 1..62: mol_type = other DNA
gtcgtatccagtgcagggtccgaggtattcgcactggatacgaccagacgcagggcttggaa

SEQ ID NO: 66 - DNA, 62 bp, synthetic construct
  source 1..62: mol_type = other DNA
gtcgtatccagtgcagggtccgaggtattcgcactggatacgacgggaccaccagtgtctgc

SEQ ID NO: 67 - DNA, 62 bp, synthetic construct
  source 1..62: mol_type = other DNA
gtcgtatccagtgcagggtccgaggtattcgcactggatacgacgtggtgggtgagacaggc

SEQ ID NO: 68 - DNA, 62 bp, synthetic construct
  source 1..62: mol_type = other DNA
gtcgtatccagtgcagggtccgaggtattcgcactggatacgactgtctggcatggctgaca

SEQ ID NO: 69 - DNA, 62 bp, synthetic construct
  source 1..62: mol_type = other DNA
gtcgtatccagtgcagggtccgaggtattcgcactggatacgacacaggcgggaaatgtgga

SEQ ID NO: 70 - DNA, 62 bp, synthetic construct
  source 1..62: mol_type = other DNA
gtcgtatccagtgcagggtccgaggtattcgcactggatacgacacgtagccagtcagcact

SEQ ID NO: 71 - DNA, 62 bp, synthetic construct
  source 1..62: mol_type = other DNA
gtcgtatccagtgcagggtccgaggtattcgcactggatacgacaggattggtgctgtgggt

SEQ ID NO: 72 - DNA, 62 bp, synthetic construct
  source 1..62: mol_type = other DNA
gtcgtatccagtgcagggtccgaggtattcgcactggatacgacagttcgctgggcagactt

SEQ ID NO: 73 - DNA, 62 bp, synthetic construct
  source 1..62: mol_type = other DNA
gtcgtatccagtgcagggtccgaggtattcgcactggatacgacagatggtgggttgccctt

SEQ ID NO: 74 - DNA, 62 bp, synthetic construct
  source 1..62: mol_type = other DNA
gtcgtatccagtgcagggtccgaggtattcgcactggatacgacaaggctgctccgtgatgt

SEQ ID NO: 75 - DNA, 62 bp, synthetic construct
  source 1..62: mol_type = other DNA
gtcgtatccagtgcagggtccgaggtattcgcactggatacgacaaagcggtgccactgtgt

SEQ ID NO: 76 - DNA, 62 bp, synthetic construct
  source 1..62: mol_type = other DNA
gtcgtatccagtgcagggtccgaggtattcgcactggatacgacgtgtggttgccaagccaa

SEQ ID NO: 77 - DNA, 62 bp, synthetic construct
  source 1..62: mol_type = other DNA
gtcgtatccagtgcagggtccgaggtattcgcactggatacgacacagctgcgaagtgccat

SEQ ID NO: 78 - DNA, 50 bp, synthetic construct; identical to SEQ ID NO: 2
  source 1..50: mol_type = other DNA

SEQ ID NO: 79 - DNA, 50 bp, synthetic construct; identical to SEQ ID NO: 3
  source 1..50: mol_type = other DNA

SEQ ID NO: 80 - DNA, 50 bp, synthetic construct; identical to SEQ ID NO: 4
  source 1..50: mol_type = other DNA

SEQ ID NO: 81 - DNA, 50 bp, synthetic construct; identical to SEQ ID NO: 5
  source 1..50: mol_type = other DNA

SEQ ID NO: 82 - DNA, 50 bp, synthetic construct; identical to SEQ ID NO: 6
  source 1..50: mol_type = other DNA

SEQ ID NO: 83 - DNA, 50 bp, synthetic construct; identical to SEQ ID NO: 7
  source 1..50: mol_type = other DNA

SEQ ID NO: 84 - DNA, 50 bp, synthetic construct; identical to SEQ ID NO: 8
  source 1..50: mol_type = other DNA

SEQ ID NO: 85 - DNA, 50 bp, synthetic construct; identical to SEQ ID NO: 9
  source 1..50: mol_type = other DNA

SEQ ID NO: 86 - DNA, 50 bp, synthetic construct; identical to SEQ ID NO: 10
  source 1..50: mol_type = other DNA

SEQ ID NO: 87 - DNA, 50 bp, synthetic construct; identical to SEQ ID NO: 11
  source 1..50: mol_type = other DNA

SEQ ID NO: 88 - DNA, 50 bp, synthetic construct; identical to SEQ ID NO: 12
  source 1..50: mol_type = other DNA

SEQ ID NO: 89 - DNA, 50 bp, synthetic construct; identical to SEQ ID NO: 13
  source 1..50: mol_type = other DNA

SEQ ID NO: 90 - DNA, 50 bp, synthetic construct; identical to SEQ ID NO: 14
  source 1..50: mol_type = other DNA

SEQ ID NO: 91 - DNA, 50 bp, synthetic construct; identical to SEQ ID NO: 15
  source 1..50: mol_type = other DNA

SEQ ID NO: 92 - DNA, 20 bp, synthetic construct
  source 1..20: mol_type = other DNA
caacgaatttggctacagca

SEQ ID NO: 93 - DNA, 20 bp, synthetic construct
  source 1..20: mol_type = other DNA
aggggagattcagtgtggtg

SEQ ID NO: 94 - DNA, 20 bp, synthetic construct
  source 1..20: mol_type = other DNA
ttgctttccttggtcaggca

SEQ ID NO: 95 - DNA, 20 bp, synthetic construct
  source 1..20: mol_type = other DNA
atccaacacttcgtggggtc

SEQ ID NO: 96 - DNA, 20 bp, synthetic construct
  source 1..20: mol_type = other DNA
gcagttgctgttcgaaagca

SEQ ID NO: 97 - DNA, 20 bp, synthetic construct
  source 1..20: mol_type = other DNA
tccaattgggagggaggact

SEQ ID NO: 98 - DNA, 18 bp, synthetic construct
  source 1..18: mol_type = other DNA
gagttccagcgcaagggt

SEQ ID NO: 99 - DNA, 18 bp, synthetic construct
  source 1..18: mol_type = other DNA
gtggggtcagtccagtgc

SEQ ID NO: 100 - DNA, 18 bp, synthetic construct
  source 1..18: mol_type = other DNA
ccggagcgtgaacagtgt

SEQ ID NO: 101 - DNA, 18 bp, synthetic construct
  source 1..18: mol_type = other DNA
ctggacagggccacaagg

SEQ ID NO: 102 - DNA, 18 bp, synthetic construct
  source 1..18: mol_type = other DNA
atggcagtcgctggacac

SEQ ID NO: 103 - DNA, 18 bp, synthetic construct
  source 1..18: mol_type = other DNA
gacaggaaacaccgccca

SEQ ID NO: 104 - DNA, 18 bp, synthetic construct
  source 1..18: mol_type = other DNA
tgcacggcagcttaacga

SEQ ID NO: 105 - DNA, 18 bp, synthetic construct
  source 1..18: mol_type = other DNA
caaggccccaaggtgagg

SEQ ID NO: 106 - DNA, 18 bp, synthetic construct
  source 1..18: mol_type = other DNA
gtaggcagcaactgggca

SEQ ID NO: 107 - DNA, 18 bp, synthetic construct
  source 1..18: mol_type = other DNA
atcctcccacgtcagcct

SEQ ID NO: 108 - DNA, 19 bp, synthetic construct
  source 1..19: mol_type = other DNA
ctgtcgcatccaccagtgt

SEQ ID NO: 109 - DNA, 18 bp, synthetic construct
  source 1..18: mol_type = other DNA
aggagcctctgacctgct

SEQ ID NO: 110 - DNA, 18 bp, synthetic construct
  source 1..18: mol_type = other DNA
ggagaacttggtggcccc

SEQ ID NO: 111 - DNA, 18 bp, synthetic construct
  source 1..18: mol_type = other DNA
actggggtgggaacaggt

SEQ ID NO: 112 - DNA, 18 bp, synthetic construct
  source 1..18: mol_type = other DNA
gggcctggctgtcgtatc

SEQ ID NO: 113 - DNA, 18 bp, synthetic construct
  source 1..18: mol_type = other DNA
tggcccagctcagtctct

SEQ ID NO: 114 - DNA, 18 bp, synthetic construct
  source 1..18: mol_type = other DNA
gcattgctgcccttctgc

SEQ ID NO: 115 - DNA, 18 bp, synthetic construct
  source 1..18: mol_type = other DNA
aggctgaagtgctcacca

SEQ ID NO: 116 - DNA, 18 bp, synthetic construct
  source 1..18: mol_type = other DNA
caggtttgaggggccagg

SEQ ID NO: 117 - DNA, 18 bp, synthetic construct
  source 1..18: mol_type = other DNA
ggtggggcctatgaggga

SEQ ID NO: 118 - DNA, 18 bp, synthetic construct
  source 1..18: mol_type = other DNA
ccgccacagctcaagtca

SEQ ID NO: 119 - DNA, 18 bp, synthetic construct
  source 1..18: mol_type = other DNA
gaccttcaccagccgctt

SEQ ID NO: 120 - DNA, 18 bp, synthetic construct
  source 1..18: mol_type = other DNA
cttcgccgacgtcctctc

SEQ ID NO: 121 - DNA, 18 bp, synthetic construct
  source 1..18: mol_type = other DNA
tgcaggttgctcacggag

SEQ ID NO: 122 - DNA, 18 bp, synthetic construct
  source 1..18: mol_type = other DNA
ccattgcccaggctgaca

SEQ ID NO: 123 - DNA, 18 bp, synthetic construct
  source 1..18: mol_type = other DNA
tgttcactctggcgctgg

SEQ ID NO: 124 - DNA, 18 bp, synthetic construct
  source 1..18: mol_type = other DNA
gtgtgccccatggagacc

SEQ ID NO: 125 - DNA, 18 bp, synthetic construct
  source 1..18: mol_type = other DNA
aagcggatggcctggttc

SEQ ID NO: 126 - DNA, 18 bp, synthetic construct
  source 1..18: mol_type = other DNA
gagtgaagctggctgggg

SEQ ID NO: 127 - DNA, 18 bp, synthetic construct
  source 1..18: mol_type = other DNA
caacgatgcctgggctca

SEQ ID NO: 128 - DNA, 18 bp, synthetic construct
  source 1..18: mol_type = other DNA
tccggagacagctgctct

SEQ ID NO: 129 - DNA, 18 bp, synthetic construct
  source 1..18: mol_type = other DNA
ccgcgtgcattctggaga

SEQ ID NO: 130 - DNA, 18 bp, synthetic construct
  source 1..18: mol_type = other DNA
aaggaagcacacgaccgg

SEQ ID NO: 131 - DNA, 18 bp, synthetic construct
  source 1..18: mol_type = other DNA
gggcaaaggctggatggt

SEQ ID NO: 132 - DNA, 18 bp, synthetic construct
  source 1..18: mol_type = other DNA
aggagcacccaacccttg

SEQ ID NO: 133 - DNA, 18 bp, synthetic construct
  source 1..18: mol_type = other DNA
ggcctgcgtcctgttctt

SEQ ID NO: 134 - DNA, 18 bp, synthetic construct
  source 1..18: mol_type = other DNA
cctgcagcaagatgggct

SEQ ID NO: 135 - DNA, 18 bp, synthetic construct
  source 1..18: mol_type = other DNA
tcccaatgctgctgccaa

SEQ ID NO: 136 - DNA, 18 bp, synthetic construct
  source 1..18: mol_type = other DNA
ccagtgggaccccgaaac

SEQ ID NO: 137 - DNA, 18 bp, synthetic construct
  source 1..18: mol_type = other DNA
aaaagctcccaccgcctc

SEQ ID NO: 138 - DNA, 18 bp, synthetic construct
  source 1..18: mol_type = other DNA
atccccgctccttcgact

SEQ ID NO: 139 - DNA, 18 bp, synthetic construct
  source 1..18: mol_type = other DNA
tcggtgctttcctgcctc

SEQ ID NO: 140 - DNA, 18 bp, synthetic construct
  source 1..18: mol_type = other DNA
ggtaccaaccgcccacaa

SEQ ID NO: 141 - DNA, 18 bp, synthetic construct
  source 1..18: mol_type = other DNA
tagctgccagtagcccga

SEQ ID NO: 142 - DNA, 18 bp, synthetic construct
  source 1..18: mol_type = other DNA
gcggcgctgcaagaatac

SEQ ID NO: 143 - DNA, 18 bp, synthetic construct
  source 1..18: mol_type = other DNA
gtgggaagcaagctccgt

SEQ ID NO: 144 - DNA, 18 bp, synthetic construct
  source 1..18: mol_type = other DNA
ccctagggaggccactgt

SEQ ID NO: 145 - DNA, 18 bp, synthetic construct
  source 1..18: mol_type = other DNA
tttgtcccacaccgaccg

SEQ ID NO: 146 - DNA, 18 bp, synthetic construct
  source 1..18: mol_type = other DNA
tgtggcccttgcactagc

SEQ ID NO: 147 - DNA, 18 bp, synthetic construct
  source 1..18: mol_type = other DNA
tgtacagcaggcggttgg

SEQ ID NO: 148 - DNA, 18 bp, synthetic construct
  source 1..18: mol_type = other DNA
ggccggctgtgtaacact

SEQ ID NO: 149 - DNA, 18 bp, synthetic construct
  source 1..18: mol_type = other DNA
actcctcggcgtcttcct

SEQ ID NO: 150 - DNA, 18 bp, synthetic construct
  source 1..18: mol_type = other DNA
ggacaaagccagtgccct

SEQ ID NO: 151 - DNA, 18 bp, synthetic construct
  source 1..18: mol_type = other DNA
gggagggcttgcatggtt

SEQ ID NO: 152 - DNA, 18 bp, synthetic construct
  source 1..18: mol_type = other DNA
tggacactgccatggctg

SEQ ID NO: 153 - DNA, 18 bp, synthetic construct
  source 1..18: mol_type = other DNA
gcgagaggagctgcgtag

SEQ ID NO: 154 - DNA, 18 bp, synthetic construct
  source 1..18: mol_type = other DNA
agcatcctgggcagcaag

SEQ ID NO: 155 - DNA, 18 bp, synthetic construct
  source 1..18: mol_type = other DNA
caatccagctgtcggcca

SEQ ID NO: 156 - DNA, 18 bp, synthetic construct
  source 1..18: mol_type = other DNA
acccacggcttcaccatg

SEQ ID NO: 157 - DNA, 18 bp, synthetic construct
  source 1..18: mol_type = other DNA
gctcgatgagtagcgcgt

SEQ ID NO: 158 - DNA, 18 bp, synthetic construct
  source 1..18: mol_type = other DNA
cgtgggagttggtggacc

SEQ ID NO: 159 - DNA, 18 bp, synthetic construct
  source 1..18: mol_type = other DNA
gcacgctccacttcacct

SEQ ID NO: 160 - DNA, 18 bp, synthetic construct
  source 1..18: mol_type = other DNA
tggaagctgacttgggcg

SEQ ID NO: 161 - DNA, 18 bp, synthetic construct
  source 1..18: mol_type = other DNA
cagacgcagggcttggaa

SEQ ID NO: 162 - DNA, 18 bp, synthetic construct
  source 1..18: mol_type = other DNA
agggtccagcgtcaaagc

SEQ ID NO: 163 - DNA, 18 bp, synthetic construct
  source 1..18: mol_type = other DNA
gggaccaccagtgtctgc

SEQ ID NO: 164 - DNA, 18 bp, synthetic construct
  source 1..18: mol_type = other DNA
cctcccggccagaacttg

SEQ ID NO: 165 - DNA, 18 bp, synthetic construct
  source 1..18: mol_type = other DNA
gtggtgggtgagacaggc

SEQ ID NO: 166 - DNA, 18 bp, synthetic construct
  source 1..18: mol_type = other DNA
cggccaggtacatcgact

SEQ ID NO: 167 - DNA, 18 bp, synthetic construct
  source 1..18: mol_type = other DNA
ccatcctccagaccgaga

SEQ ID NO: 168 - DNA, 22 bp, synthetic construct
  source 1..22: mol_type = other DNA
gaaaggaaaggcatcactatgg

SEQ ID NO: 169 - DNA, 22 bp, synthetic construct
  source 1..22: mol_type = other DNA
agacaccggatctatttgcatt

SEQ ID NO: 170 - DNA, 18 bp, synthetic construct
  source 1..18: mol_type = other DNA
ttggcccagacccaacta

SEQ ID NO: 171 - DNA, 18 bp, synthetic construct
  source 1..18: mol_type = other DNA
gcaggaggcgttgtcatt

SEQ ID NO: 172 - DNA, 20 bp, synthetic construct
  source 1..20: mol_type = other DNA
caggaattcatggcctttgt

SEQ ID NO: 173 - DNA, 20 bp, synthetic construct
  source 1..20: mol_type = other DNA
gctggaaagctcagctccta

SEQ ID NO: 174 - DNA, 20 bp, synthetic construct
  source 1..20: mol_type = other DNA
tgtttgcaagcaggactttg

SEQ ID NO: 175 - DNA, 20 bp, synthetic construct
  source 1..20: mol_type = other DNA
tggcaccaaaggattcctaa

SEQ ID NO: 176 - DNA, 20 bp, synthetic construct
  source 1..20: mol_type = other DNA
cacctgtgcctctcagaaca

SEQ ID NO: 177 - DNA, 20 bp, synthetic construct
  source 1..20: mol_type = other DNA
tgaggaaagctccaacaacc

SEQ ID NO: 178 - DNA, 20 bp, synthetic construct
  source 1..20: mol_type = other DNA
ccacctccagcttgtacctg

SEQ ID NO: 179 - DNA, 20 bp, synthetic construct
  source 1..20: mol_type = other DNA
gagcagagaatccgaggacg

SEQ ID NO: 180 - DNA, 18 bp, synthetic construct
  source 1..18: mol_type = other DNA
ctgagtgccgccttgtga

SEQ ID NO: 181 - DNA, 18 bp, synthetic construct
  source 1..18: mol_type = other DNA
aaccgcagtacttgggcc

SEQ ID NO: 182 - DNA, 18 bp, synthetic construct
  source 1..18: mol_type = other DNA
tgcagagccccaagaagc

SEQ ID NO: 183 - DNA, 18 bp, synthetic construct
  source 1..18: mol_type = other DNA
gaggttgtggcggatgct

SEQ ID NO: 184 - DNA, 18 bp, synthetic construct
  source 1..18: mol_type = other DNA
agggccgtgtgactttcg

SEQ ID NO: 185 - DNA, 18 bp, synthetic construct
  source 1..18: mol_type = other DNA
agcagccaccgtgagttc

SEQ ID NO: 186 - DNA, 18 bp, synthetic construct
  source 1..18: mol_type = other DNA
gccacggccaacaaacag

SEQ ID NO: 187 - DNA, 18 bp, synthetic construct
  source 1..18: mol_type = other DNA
tgtcctccccgcagatga

SEQ ID NO: 188 - DNA, 18 bp, synthetic construct
  source 1..18: mol_type = other DNA
gttcacccaggaccctgc

SEQ ID NO: 189 - DNA, 18 bp, synthetic construct
  source 1..18: mol_type = other DNA
ctgctttgggggcttcga

SEQ ID NO: 190 - DNA, 18 bp, synthetic construct
  source 1..18: mol_type = other DNA
gtgacttgcctcccggac

SEQ ID NO: 191 - DNA, 18 bp, synthetic construct
  source 1..18: mol_type = other DNA
cttcgttccgcctggtgt

SEQ ID NO: 192 - DNA, 18 bp, synthetic construct
  source 1..18: mol_type = other DNA
ggggactcgggagcctaa

SEQ ID NO: 193 - DNA, 18 bp, synthetic construct
  source 1..18: mol_type = other DNA
gtggacctcacactcgcc

SEQ ID NO: 194 - DNA, 18 bp, synthetic construct
  source 1..18: mol_type = other DNA
cgcagtaccactccctgc

SEQ ID NO: 195 - DNA, 18 bp, synthetic construct
  source 1..18: mol_type = other DNA
cggcatggtggtggatgt

SEQ ID NO: 196 - DNA, 18 bp, synthetic construct
  source 1..18: mol_type = other DNA
gaaccctgctccttcggg

SEQ ID NO: 197 - DNA, 18 bp, synthetic construct
  source 1..18: mol_type = other DNA
agcacagcggcagtgatt

SEQ ID NO: 198 - DNA, 18 bp, synthetic construct
  source 1..18: mol_type = other DNA
atcactgccgctgtgctt

SEQ ID NO: 199 - DNA, 18 bp, synthetic construct
  source 1..18: mol_type = other DNA
ggaaaggtgctgctgggt

SEQ ID NO: 200 - DNA, 18 bp, synthetic construct
  source 1..18: mol_type = other DNA
ctccacccacccaagcag

SEQ ID NO: 201 - DNA, 18 bp, synthetic construct
  source 1..18: mol_type = other DNA
acgctgtcctgcaatgct

SEQ ID NO: 202 - DNA, 18 bp, synthetic construct
  source 1..18: mol_type = other DNA
aaccgcaccatggtggag

SEQ ID NO: 203 - DNA, 18 bp, synthetic construct
  source 1..18: mol_type = other DNA
ctccgagggctaccacct

SEQ ID NO: 204 - DNA, 18 bp, synthetic construct
  source 1..18: mol_type = other DNA
ccccaggcagttcccaag

SEQ ID NO: 205 - DNA, 18 bp, synthetic construct
  source 1..18: mol_type = other DNA
ctttgggaagagccgggg

SEQ ID NO: 206 - DNA, 20 bp, synthetic construct
  source 1..20: mol_type = other DNA
agctgtgtatggaccagtgc

SEQ ID NO: 207 - DNA, 20 bp, synthetic construct
  source 1..20: mol_type = other DNA
atgaggacctgcagcatgtc

SEQ ID NO: 208 - DNA, 20 bp, synthetic construct
  source 1..20: mol_type = other DNA
tcctctgaaaaggcagcagg

SEQ ID NO: 209 - DNA, 20 bp, synthetic construct
  source 1..20: mol_type = other DNA
gcatgccacagaaggactct

SEQ ID NO: 210 - DNA, 18 bp, synthetic construct
  source 1..18: mol_type = other DNA
tgcggctttacccacctt

SEQ ID NO: 211 - DNA, 18 bp, synthetic construct
  source 1..18: mol_type = other DNA
tgtctggcatggctgaca

SEQ ID NO: 212 - DNA, 18 bp, synthetic construct
  source 1..18: mol_type = other DNA
acagctggcttactggct

SEQ ID NO: 213 - DNA, 18 bp, synthetic construct
  source 1..18: mol_type = other DNA
acaggcgggaaatgtgga

SEQ ID NO: 214 - DNA, 18 bp, synthetic construct
  source 1..18: mol_type = other DNA
tggtttttaggggcccca

SEQ ID NO: 215 - DNA, 18 bp, synthetic construct
  source 1..18: mol_type = other DNA
acgtagccagtcagcact

SEQ ID NO: 216 - DNA, 18 bp, synthetic construct
  source 1..18: mol_type = other DNA
aatccaagcctcacccca

SEQ ID NO: 217 - DNA, 18 bp, synthetic construct
  source 1..18: mol_type = other DNA
aggattggtgctgtgggt

SEQ ID NO: 218 - DNA, 18 bp, synthetic construct
  source 1..18: mol_type = other DNA
aagatggcggctgctgta

SEQ ID NO: 219 - DNA, 18 bp, synthetic construct
  source 1..18: mol_type = other DNA
agttcgctgggcagactt

SEQ ID NO: 220 - DNA, 18 bp, synthetic construct
  source 1..18: mol_type = other DNA
acattatccggcagccct

SEQ ID NO: 221 - DNA, 18 bp, synthetic construct
  source 1..18: mol_type = other DNA
agatggtgggttgccctt

SEQ ID NO: 222 - DNA, 18 bp, synthetic construct
  source 1..18: mol_type = other DNA
acgagtgtgcgtgagtgt

SEQ ID NO: 223 - DNA, 18 bp, synthetic construct
  source 1..18: mol_type = other DNA
aaggctgctccgtgatgt

SEQ ID NO: 224 - DNA, 18 bp, synthetic construct
  source 1..18: mol_type = other DNA
gcctttgcttcgtgctga

SEQ ID NO: 225 - DNA, 18 bp, synthetic construct
  source 1..18: mol_type = other DNA
aaagcggtgccactgtgt

SEQ ID NO: 226 - DNA, 18 bp, synthetic construct
  source 1..18: mol_type = other DNA
tgaggagcaagcgagcaa

SEQ ID NO: 227 - DNA, 18 bp, synthetic construct
  source 1..18: mol_type = other DNA
gtgtggttgccaagccaa

SEQ ID NO: 228 - DNA, 18 bp, synthetic construct
  source 1..18: mol_type = other DNA
ttttgccgcctagtgcca

SEQ ID NO: 229 - DNA, 18 bp, synthetic construct
  source 1..18: mol_type = other DNA
acagctgcgaagtgccat

SEQ ID NO: 230 - DNA, 62 bp, synthetic construct
  source 1..62: mol_type = other DNA
gtcgtatccagtgcagggtccgaggtattcgcactggatacgactggcccagctcagtctct